(19) United States
(12) Patent Application Publication
(10) Pub. No.: US 2013/0064815 A1
(43) Pub. Date: Mar. 14, 2013

(54) INDUCING APOPTOSIS IN QUIESCENT CELLS

(71) Applicant: Hilary Coller, Princeton, NJ (US)

(72) Inventor: Hilary Coller, Princeton, NJ (US)

(73) Assignee: THE TRUSTEES OF PRINCETON UNIVERSITY, Princeton, NJ (US)

(21) Appl. No.: 13/612,199

(22) Filed: Sep. 12, 2012

Related U.S. Application Data
(60) Provisional application No. 61/533,598, filed on Sep. 12, 2011.

(51) Int. Cl. A61K 31/496; C40B 30/04; A61P 35/00; A61K 39/395

(52) U.S. Cl. 424/133.1; 514/252.18; 506/9

(57) ABSTRACT

Compositions comprising an autophagy inhibitor and at least one of an NADPH modulator or a glutathione inhibitor are provided. Methods of inhibiting or killing a quiescent cell are provided. Methods of treating cancer are provided. Methods of identifying compositions that inhibit or kill quiescent cells are provided. Methods of identifying compositions that inhibit or kill quiescent cells are provided. Methods of inducing apoptosis are provided. Methods of sensitizing quiescent cells to proteasome inhibitors are provided.

This application claims the benefit of U.S. Provisional Application No. 61/533,598, filed Sep. 12, 2011, which is incorporated herein by reference as if fully set forth.

This invention was made with government support under National Institutes of Health Grants #CA128620, #CA147961 and #AI078063. The government has certain rights in this invention.

The sequence listing electronically filed with this application titled “Sequence Listing,” which was created on Sep. 12, 2012, and had a size of 17,650 bytes is incorporated by reference herein as if fully set forth.

### FIELD OF INVENTION

The disclosure herein relates to inhibition or killing of quiescent cells.

### BACKGROUND

Lymphocytes undergo a major metabolic shift upon transitioning between proliferation and quiescence. Early studies showed that lectin stimulation of lymphocytes led to increased glucose uptake, and an increased rate of glycolysis and pentose phosphate pathway activities. More recent experiments have focused on a murine pro-B-cell lymphoid cell line FL5.12 which proliferates in response to the cytokine interleukin IL-3. IL-3 stimulation results in an 8-fold increased glycolytic flux. IL-3 also induces the cells to consume less oxygen per glucose consumed, and excrete much more lactate, indicating a shift away from oxidative towards glycolytic metabolism. For human peripheral blood T lymphocytes, stimulation resulted in a 30-fold increase in glycolysis; for thymocytes, the increase was 50-fold. These differences in quiescent and proliferating lymphocytes have played a role in our understanding of the quiescent state, and experiments with lymphocytes as a model system have been important contributors to the widely-held belief that quiescence is characterized by decreased metabolic activity.

Most cytotoxins and anti-cancer agents target proliferating cells, based on the fact that they are proliferating. However, little is known about how cells can achieve quiesence or what contributes to a cell's viability during quiescence.

### SUMMARY

In an aspect, the invention relates to a composition. The composition includes an autophagy inhibitor and at least one of an NADPH modulator or a glutathione modulator.

In an aspect, the invention relates to a method of inhibiting or killing a quiescent cell comprising exposing the quiescent cell to an autophagy inhibitor and at least one of an NADPH modulator or a glutathione modulator.

In an aspect, the invention relates to a method of treating cancer. The method includes administering an autophagy inhibitor and at least one of an NADPH modulator or a glutathione modulator to a cancer patient.

In an aspect, the invention relates to a method of identifying compositions that inhibit or kill quiescent cells. The method includes identifying a target by analyzing at least one of the metabolic flux, gene expression, protein expression, microRNA content, histone modification, signaling pathway activity, or physiology of quiescent cells. The method also includes identifying a candidate inhibitor of the target, and exposing the quiescent cells to the candidate inhibitor. The method also includes identifying whether the candidate inhibitor inhibits or kills quiescent cells.

In an aspect, the invention relates to a method of identifying compositions that inhibit or kill quiescent cells. The method includes exposing a quiescent cell to a candidate inhibitor and monitoring the physiology of the quiescent cell.

In an aspect, the invention relates to a method of inducing apoptosis. The method includes exposing at least one of a cell, a cell culture, a tissue, an organ, an organism or a human to an autophagy inhibitor and at least one of an NADPH modulator or a glutathione modulator.

In an aspect, the invention relates to a method of sensitizing quiescent cells to proteasome inhibitors. The method includes exposing at least one of a cell, a cell culture, a tissue, an organ, an organism or a human to an autophagy inhibitor and at least one of an NADPH modulator or a glutathione modulator.

In an aspect, the invention relates to a composition comprising DHEA and an autophagy inhibitor.

In an aspect, the invention relates to a method of inhibiting or killing a quiescent cell. The method includes exposing the quiescent cell to DHEA and an autophagy inhibitor.

In an aspect, the invention relates to a method of treating cancer comprising administering DHEA and an autophagy inhibitor to a cancer patient.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description of the preferred embodiment of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIG. 1 illustrates an experimental design for metabolomic studies. The experimental design includes four steps: 1) separate by polarity; 2) ionize; 3) separate and identify by molecular weight; and 4) quantify.

FIG. 2A illustrates induction of apoptosis in serum starved fibroblasts with a combination of DHEA and bafilomycin. The squares illustrate proliferating (P), the triangles illustrate 7-day contact inhibited (7 dCI) and the circles illustrate 4-days serum starvation (4 dSS). FIG. 2B illustrates induction of apoptosis in serum starved fibroblasts with HCQ. The squares illustrate proliferating (P), the triangles illustrate 7-day contact inhibited (7 dCI) and the circles illustrate 4-days serum starvation (4 dSS).

FIGS. 3A-D illustrate induction of quiescence. FIG. 3A illustrates proliferating (P), 7-day contact-inhibited (CI7), 14-day contact-inhibited (CI14), and contact-inhibited and serum-deprived (CI14SS7) fibroblasts stained with propidium iodide and analyzed for cell cycle distribution with flow cytometry. The fraction of cells in G0/G1 increased in quiescent cells. From left to right in each bar graph, the bars illustrate G1/G0, S and G2. FIG. 3A also illustrates images of proliferating (P), 7-day contact-inhibited (CI7), 14-day contact-inhibited (CI14), and contact-inhibited and serum-deprived (CI14SS7) below the respective distribution graphs. FIG. 3B illustrates levels of p27 or GAPDH in lysates obtained from fibroblasts induced into quiescence by contact inhibition or serum starvation. Lysates were collected over a timecourse and analyzed by immunoblotting with an antibody to p27^{Kip1}. p27^{Kip1} levels were induced in cells made quiescent by either antiproliferative signal. FIGS. 3C and D illustrate analysis of fibroblasts with pyronin Y and Hoechst 33342. Proliferating and quiescent fibroblasts were stained with pyronin Y and Hoechst 33342 and analyzed by flow cytometry (FIG. 3D). The fraction of cells with low pyronin Y content increased in fibroblasts induced into quiescence by multiple methods (FIG. 3C). In FIG. 3C, the top portion of each bar illustrates G2/M, the second from top illustrates S, the third from top illustrates G1, and the bottom illustrates G0. Each portion is separated by a horizontal line through the bar.

FIGS. 4A-C illustrate that glycolytic rates are similar in proliferating and quiescent fibroblast. FIG. 4A shows, in four panels, the amount of glucose, lactate, glutamine and glutamate measured in conditioned medium from P, CI7, CI14 and CI14SS7 cells. Data are from three experiments with five replicates at each timepoint, and error bars indicate standard error. From left to right in each panel, the bars represent P, CI7, CI14 and CI14SS7. FIG. 4B shows metabolite pool sizes in 6 panels (Hexose-P, FBP, DHAP, 3PG, PEP, Pentose-P). Metabolites were extracted from cells in different proliferative states and the levels of specific metabolites were quantified using mass spectrometry. Metabolite levels in individual samples were normalized to protein content at the time of harvest. Means from four experiments each containing 4 or 5 replicates are shown. Error bars indicate standard error. With a FDR of 0.05, none of the metabolite levels are different between the P, CI7 and CI14 cells. From left to right, the bars represent in each panel P, CI7, CI14 and CI14SS7. FIG. 4C illustrates isotope-labeling dynamics of glycolysis in proliferating (P), 7-day contact-inhibited (CI7) and 14-day contact-inhibited (CI14) fibroblasts. Medium was changed to [U-¹³C]-glucose at time zero and the fraction of each metabolite that is ¹³C labeled was determined at the indicated times after switching to labeled medium. Data are pooled from 5 experiments and error bars indicate standard deviations.

FIGS. 5A-E illustrate glycolytic rates in proliferating and quiescent fibroblasts. FIG. 5A shows rates of glucose consumption, lactate excretion, glutamine consumption and glutamate excretion (in four panels) monitored in P, CI7, CI14, CI14SS7, SS4, SS7, P low glucose/low glutamine, and CI14 low glucose/low glutamine fibroblasts using the YSI-7100 bioanalyzer. Levels were normalized for the amount of cellular protein present during the conditioning time. Error bars indicate standard error. From left to right in each panel, the bars represent P, CI7, CI14SS7, SS4, P low glc gln and CI14 low glc gln. FIGS. 5B-E show representative plots of metabolite levels over time used to determine the reported rates (B, P and CI7; C, CI14 and CI14SS7; D, SS4 and SS7; and E, P Low Gluc Low Gln and C14 Low Gluc Low Gln).

FIG. 6 illustrates basal metabolites in P, CI7 and CI14 fibroblasts. Metabolites were analyzed using LC-MS/MS. Individual metabolite levels were normalized for protein content. The log (base 2) of the ratio of CI7 or CI14 to the average of P metabolite levels over all experiments was determined for each sample. Means from four experiments each containing 4 or 5 replicates are shown. Error bars indicate standard error.

FIGS. 7A-B illustrate a flux-balanced model of central carbon metabolism; an ODE-based model of central carbon metabolism was developed to describe the time-dependent metabolic labeling. FIG. 7A shows a schematic of fluxes in the model. F₀ to F₁₂ represent the unknown fluxes, except for F₉, which is the latent hexose-phosphate pool. A, B, C, and D are the uptake and excretion rates. X, Y and Z are dependent parameters of the above fluxes and pool sizes, whose expressions are determined by balancing all the relevant fluxes. X is the protein synthesis rate, Y is the anaplerotic flux from pyruvate, and Z is the net flux from malate to oxaloacetate. FIG. 7B shows conversion of the isotopically labeled metabolic forms in the glucose and glutamine labeling experiments. The numbers under the metabolite names represent the positions at which a metabolite is labeled (“0” means an unlabeled metabolite). Low-abundance isotope-labeled forms, such as 1×¹³C-citrate, were excluded from the model.

FIGS. 8A-8AB illustrate modeling results for central carbon metabolism. FIGS. 8A, B, C, and D show model fits for metabolites in P fibroblasts. FIGS. 8E, F, G, and H show model fits for metabolites in CI7 fibroblasts. FIGS. 8I, J, K, and L show model fits for metabolites in CI14 fibroblasts. FIGS. 8M, N, O, and P show model fits for metabolites in CI14SS7 fibroblasts. For each of FIGS. 8A-O, experimentally-measured concentrations of different labeled and unlabeled metabolites (mean+/−1 SD) at the indicated time points are plotted against the model predictions (smooth curves) from a typical flux solution set. The time-axis is in logarithmic scale to better illustrate the samples at early time points. Data and simulated results for [U-¹³C]-glucose labeling experiments are labeled by the metabolite name only. For the [U-¹³C]-glutamine labeling, a “Q” precedes the metabolite name. FIGS. 8Q,R and S show histograms of the distribution of consistent fluxes in each condition for P fibroblasts. FIGS. 8T,U and V show histograms of the distribution of consistent fluxes in each condition for CI7 fibroblasts. FIGS. 8W, X and Y show histograms of the distribution of consistent fluxes in each condition for CI14 fibroblasts. FIGS. 8Z,AA and AB show histograms of the distribution of consistent fluxes in each condition for CI14SS7 fibroblasts. For each of FIGS. 8Q-AB, the x-axis indicates the flux values (in logarithmic scale); the y-axis is the number of counts (within the 1000 consistent solution sets) that have a specific flux value. The resultant solution distribution provides a representation of the fluxes that are potentially consistent with the observed laboratory data in each cell state.

FIG. 9 compares central metabolic fluxes in proliferating (P) and 14-day contact-inhibited (CI14) fibroblasts. Fluxes were derived by computational integration of all available experimental data within a systems-level, flux-balanced metabolic model. Arrow size indicates the magnitude of the flux in CI14 fibroblasts. Relative rates compared to P fibroblasts: a higher flux in CI14 fibroblasts was seen in citrate to ΔKG and the reverse (2×), fatty acids to acetyl-CoA (2×) FBP to DHAP and the reverse (2×), and pyruvate to OAA (>4×) and a higher flux in proliferating fibroblasts was seen in Pentose P to ATP (½×), ATP to nucleic acid (½×) and pyruvate to acetyl-CoA (<¼×). While the RibP to UTP flux is mostly faster (within the 1,000 identified solutions) for proliferating versus quiescent fibroblasts, its distributions do overlap across different proliferative states. Thus, a stringent condition for different rates is not met for the RibP to UTP flux.

FIGS. 10A-C show that the pentose phosphate pathway (PPP) is active in quiescent fibroblasts. FIG. 10A illustrates isotope-labeling dynamics in the PPP for proliferating, 7-day contact-inhibited (CI7) and 14-day contact-inhibited (CI14) fibroblasts. The fraction of fully labeled ribose-phosphate (left) or sedoheptulose-7-phosphate (right) after addition of [U-¹³C]-glucose is plotted for cells in each condition at each time-point. Similarly, the fraction of ATP and UTP with five ¹³C-atoms is plotted. The 5×¹³C-ATP and 5×¹³C-UTP are uniformly labeled in their ribose portion and unlabeled in the base portion, as confirmed by MS/MS analysis. Data are pooled from 5 experiments and error bars indicate standard deviation. FIG. 10B shows a schematic diagram of lactate labeling from [1,2-¹³C]-glucose. [1,2-¹³C]-glucose is converted into 2×¹³C-lactate through the canonical glycolysis pathway and 1×¹³C-lactate through the PPP. FIG. 10C shows the results when fibroblasts in different proliferative conditions were incubated with [1,2-¹³C]-glucose for 4 hours. Levels of 1×¹³C-lactate and 2×¹³C-lactate were monitored with mass spectrometry. The ratio of 1×¹³C-lactate to 2×¹³C-lactate is plotted for fibroblasts in each condition. Means+/−1 standard error (n=4) are shown. Asterisks indicate p-value<0.01 (P vs. CI7, p=0.006 and P vs. CI14 fibroblasts p=0.002 by student's t-test). Bars from left to right illustrate P, CI7 and CI14.

FIGS. 11A shows that pentose phosphate pathway (PPP) enzymes are induced and the fraction of reduced glutathione is enhanced in quiescent fibroblasts. FIG. 11A illustrates immunoblots of proliferating and quiescent fibrolasts. Protein levels of glucose-6-phosphate dehydrogenase (G6PD) and 6-phosphogluconate dehydrogenase (PGD), both of which generate NADPH, were monitored. GAPDH was monitored as a loading control.

FIGS. 12A-B show that the pentose phosphate pathway contributes to the survival of quiescent fibroblasts. FIG. 12A illustrates analysis of proliferating or 14-day contact-inhibited (CI14) fibroblasts treated with DMSO control (left panel), 100 μM DHEA (middle panel) or 250 μM DHEA (right panel) for 4 days. Cells were incubated with PI and analyzed by flow cytometry. Data are from four independent experiments, error bars indicate standard error. For CI14 vs P (no treatment), p=0.113. For CI14 vs P (100 μM DHEA), p=0.0012. For CI14 vs P (250 μM DHEA), p=0.0011. Left bar illustrates P and right bar illustrates CI14. FIG. 12B illustrates analysis of Proliferating (P), 7-day contact-inhibited (CI11), or 3-day serum-starved (SS7) fibroblasts treated with ethanol vehicle control or varying amounts of DHEA dissolved in ethanol for four days. Cells were analyzed for caspase-3/7 activity by monitoring luminescence emission of a caspase-3/7 substrate. Data were normalized to the vehicle control. For P versus CI11 cells, results are an average of 4 experiments with 2 or 3 replicates, error bars represent standard error. Normalized caspase-3/7 activity in CI11 and P cells were statistically significantly different at all doses except 100 μM. For P versus SS7 cells, data represent three experiments with three replicates in each. Normalized caspase activity in SS7 and P cells were statistically significantly different at all doses.

FIG. 13 shows that a truncated TCA cycle exists in proliferating but not contact-inhibited fibroblasts. Proliferating (P), 7-day contact-inhibited (CI7) and 14-day contact-inhibited (CI14) fibroblasts were switched from unlabeled to [U-¹³C]-glucose at time zero. The graphs show the fractional incorporation of ¹³C into the indicated metabolites over time. Data represent averages from three experiments and error bars indicate standard deviation. Note the minimal labeling of α-ketoglutarate and succinate in the proliferating cells.

FIGS. 14A-C illustrate that glutamine drives both clockwise and counter-clockwise flux through the TCA cycle. FIG. 14A illustrates from left to right, analysis of proliferating (P), 7-day contact-inhibited (CI7) or 14-day contact-inhibited (CI14) fibroblasts incubated with [U-¹³C]-glutamine. Metabolites were harvested and their extent of labeling measured by LC-MS/MS. Ketoglutarate in the TCA cycle can be converted to succinate in the clockwise (or “forward”) direction or converted to citrate in the counter-clockwise (or “reverse”). The only known route to 5×¹³C-citrate is via this “reverse” flux from α-ketoglutarate. 5×¹³C-Citrate can then be converted to 3×¹³C-malate by ATP-citrate lyase to produce acetyl-CoA to drive fatty acid biosynthesis. Data represent the average of four experiments. Error bars indicate standard deviations. FIG. 14B illustrates that IDH1 is upregulated at the transcript and protein level in quiescent fibroblasts. Transcript levels of IDH1 were monitored in two independent experiments (indicated with subscripts) of P, CI7 and CI14 fibroblasts by microarray (left panel). Data are shown in a heatmap format (left panel) with elevated expression in quiescent cells shown for IDH1 and decreased expression in quiescent cells in IDH2, IDH3A and IDH3B. Results are shown for multiple isocitrate dehydrogenase isozymes. Protein levels for isocitrate dehydrogenase 1 (IDH1), a metabolic enzyme that synthesizes the conversion of isocitrate to α-ketoglutarate and thereby generates NADPH, were monitored by immunoblotting (right panel). GAPDH was monitored as a loading control. FIG. 14C illustrates analysis of P, CI7, CI14 and CI14SS7 fibroblasts incubated with [U-¹⁴C]-glutamine for 24 hours. Fatty acids were extracted and ¹⁴C incorporation was determined by scintillation counting and normalized for the amount of protein present. Error bars indicate standard error and p values were determined with the student's t-test. For CI7 vs P, p=0.0025; for CI14 vs P, p=0.0184; for CI14SS7 vs P, p=0.0001. Bars illustrate, from left to right, P, CI7, CI14 and CI14CSS7.

FIG. 15 shows that labeled glutamate levels decrease with time after switching into [U-¹³C]-glutamine in CI7 and CI14 but not P fibroblasts. P, CI7 or CI14 fibroblasts were switched from unlabeled medium to medium containing [U-¹³C]-glutamine and the fraction of fully labeled glutamate (left plot) and unlabeled glutamate (right plot) was determined over time. Results are an average of four experiments and error bars indicate standard deviations.

FIG. 16 shows that contact-inhibited fibroblasts secrete high levels of specific extracellular matrix proteins. Four-day conditioned medium was collected from proliferating (P) and 14-day contact-inhibited (CI14) fibroblasts conditioned with either no serum or 0.1% serum, and with 0.03% platelet derived growth factor (PDGF-BB) for proliferating cells. The amount of conditioned medium was normalized to the change in protein content over time. Conditioned medium was precipitated and immunoblotted with an antibody to fibronectin, collagen (col21a1) or laminin (lama2).

FIGS. 17A-B show the results of treatment using DHEA and/or bafilomycin in combination with the proteasome inhibitor bortezomib. The chart shows the results of treatment with 425 uM DHEA in varying concentrations with bortezomib (diamonds), treatment with 100 nM bafilomycin with varying concentrations of bortezomib (squares with an X), and treatment with 200 nM bafilomycin with varying concentrations of bortezomib (squares).

FIGS. 18A-18B illustrate the induction of an NADPH production program in quiescent fibroblasts. Bars illustrate, from left to right, P, 7 dCI, 14 dCI, 14 dCI17 dSS, 4 dSS and 7 dSS.

FIG. 19 illustrates that quiescent fibroblasts have more reduced glutathione which is reduced by DHEA. The left bar illustrates P and right bar illustrates 7 dSS.

FIG. 20 illustrates that DHEA treatment results in more oxidized protein in quiescent fibroblasts.

FIGS. 21A-21D illustrate that autophagy is induced in quiescent fibroblasts.

FIGS. 22A-22C illustrate that inhibiting autophagy results in more oxidized and nitrosylated protein in quiescent fibroblasts.

FIGS. 23A-23C illustrates that autophagy is induced in quiescent fibroblasts in vivo.

FIGS. 24A-C illustrate that quiescent fibroblasts are protected from proteasome inhibition.

FIGS. 25A-25D illustrate that autophagy inhibition sensitizes quiescent fibroblasts to proteasome inhibition. In FIG. 25D, grey represents results for MG132 alone, and black represents results with MG132 and bafilomycin.

FIGS. 26A-26D illustrate that superoxide dismutase inhibitors sensitize quiescent fibroblasts to proteasome inhibition.

### DETAILED DESCRIPTION OF THE PREFERRED EMBODIMENTS

Certain terminology is used in the following description for convenience only and is not limiting. The following abbreviations are used: CI7, contact-inhibited 7 days; CI14, contact-inhibited 14 days; CI14SS7, contact inhibited 14 days and serum-starved 7 days; DHAP, dihydroxyacetone-phosphate; DHEA, dehydroepiandrosterone; FBP, fructose-1,6-bisphosphate; G6PD, glucose-6-phosphate dehydrogenase; IDH1, isocitrate dehydrogenase 1; ODE, ordinary differential equations; P, proliferating; PBS, phosphate-buffered saline; PBS-T, phosphate buffered saline containing 0.1% Tween-20; PEP, phosphoenolpyruvate; PGD, 6-phosphogluconate dehydrogenase; PI, propidium iodide; PPP, pentose phosphate pathway; SS4, serum-starved 4 days; SS7, serum-starved 7 days; TBS, tris-buffered saline; TBS-T, tris-buffered saline containing 0.1% Tween-20; TCA, tricarboxylic acid; U, universal.

The words “a,” and, “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

Embodiments include compositions comprising an autophagy inhibitor and at least one of an NADPH modulator or a glutathione modulator.

NADPH modulators may include any agent that reduces NADPH levels in a cell, including a pentose phosphate pathway inhibitor, inhibitors of the novel quiescent fibroblast NADPH production program pathway (example 21, below), and inhibitors of NADPH-generating reactions. NADPH modulators may include but are not limited to an inhibitor of glucose-6-phosphate dehydrogenase, an inhibitor of 6 phosphogluconate dehydrogenase, an inhibitor of isocitrate dehydrogenase 1, an inhibitor of isocitrate dehydrogranse 2, an inhibitor of an enzyme in the pentose phosphate pathway, dehydroepiandrosterone, 16α-fluoro-5-androsten-17-one, 16α-1-fluoro-5α-androstan-17-one, 3-β-methylandrost-5-en-17-one, somatostatin, a peptide of hypothalamic origin, an inhibitor of transketolase, an analog of a tranketolase inhibitor, a thiamine analog, oxythiamine, a non-charged thiamine analog, micronized DHEA, DHEA, an siRNA targeting a pentose phosphate pathway enzyme, an siRNA targeting gluocse-6-phosphate dehydrogenase, an siRNA targeting nrf2, an siRNA targeting srbp, an shRNA targeting a pentose phosphate pathway enzyme, an shRNA targeting gluocse-6-phosphate dehydrogenase, an shRNA targeting nrf2, and an shRNA targeting srbp. The NADPH modulator may include a vector or virus encoding any of the aforementioned peptides, proteins, or RNAs. The NADPH modulator may be an analog or precursor of any of the aforementioned compounds (including any agent in the list, whether small molecule, protein, RNA or other). The NADPH modulator may include a combination of any two or more of the aforementioned compounds or include a pharmaceutically acceptable salt of any of the foregoing substances. In an embodiment, DHEA is the NADPH modulator. DHEA may assert its affect described herein by modulating levels of NADPH, or possibly by other mechanisms. Regardless of mechanism of action, DHEA is referred to herein as an NADPH modulator and may be included in compositions and methods herein that include an “at least one of an NADPH modulator and a glutathione modulator.”

Glutathione modulators may include any agent that inhibits glutathione biosynthesis or reduces the amount of glutathione in the cell. Glutathione inhibitors may include butathione sulfoximine. An NADPH modulator may also affect glutathione production.

As used herein, “at least one of an NADPH modulator and a glutathione modulator” refers to at least one of the NADPH modulator or the glutathione modulator as described herein generically and by reference to specific substances, and also to agents that may act as both an NADPH modulator and a glutathione modulator.

Inhibitors of autophagy may include but are not limited to a macrolide antibiotic, bafilomycin, concanamycin, an inhibitor of vacuolar type H+-ATPase, an inhibitor of lysosomal acidification, an antimalarial substance, chloroquine, hydroxychloroquine, micronized hydroxychloroquine, quinacrine, an analog of a macrolide antibiotic, an analog of bafilomycin, chloroquine analogs having a lateral alkyl side chain and dialkyl substitution on the lateral side chain, 7-chloro-N-(3-(4-(7-trifluoromethyl)quinolin-4-yl)piperazin-1-yl)propyl)quinolin-4-amine, {3-[4-(7-chloro-quinolin-4-yl)-piperazin-1-yl]-propyl}-(7-rifluoromethyl-quinolin-4-yl)-amine, 3-methyladenine, an siRNA targeting a protein in the autophagy pathway, an shRNA targeting a protein within the autophagy pathway, an siRNA targeting atg5, an siRNA targeting atg7, an siRNA targeting lc3/atg8, an siRNA targeting beclin1, an shRNA targeting atg5, an shRNA targeting atg7, an shRNA targeting lc3/atg8, and an shRNA targeting beclin 1. The autophagy inhibitor may include a vector or virus encoding any of the aforementioned peptides, proteins, or RNAs. The autophagy inhibitor may include an analog or precursor of any of the aforementioned compounds (including any agent in the list whether small molecule, protein, RNA or other). The autophagy inhibitor may include two or more of any two or more of the aforementioned compounds or include a pharmaceutically acceptable salt of any of the foregoing substances. In an embodiment, the autophagy inhibitor is bafilomycin.

Embodiments include a composition comprising a micronized DHEA or a pharmaceutically acceptable salt thereof as the NADPH modulator and a micronized hydroxychloroquine or a pharmaceutically acceptable salt thereof as the autophagy inhibitor.

Embodiments may include a composition comprising an autophagy inhibitor and at least one of an NADPH modulator or a glutathione modulator, and an anti-cancer chemotherapeutic agent or a pharmaceutically acceptable salt thereof other than the autophagy inhibitor and other than the at least one of an NADPH modulator or a glutathione modulator and other than the autophagy inhibitor. The anti-cancer chemotherapeutic agent may be but is not limited to at least one of oxaliplatin, capecitabine, bevacizumab, docetaxel, paclitaxel, carboplatin, ixabepilone, androstenedione, or testosterone.

Embodiments may include a composition comprising an autophagy inhibitor and at least one of an NADPH modulator or a glutathione modulator, and a targeting agent adapted to deliver at least one of the NADPH modulator or the autophagy inhibitor to a tumor cell. A targeting agent may include any one or more of the agents described for tumor targeting in Example 9, below. Compositions including an autophagy inhibitor and at least one of an NADPH modulator or a glutathione modulator herein may further include a pharmaceutically acceptable carrier. Pharmaceutically acceptable carriers that may be a part of a composition herein include but are not limited to at least one of ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, talc, magnesium carbonate, kaolin, non-ionic surfactants, edible oils, physiological saline, bacteriostatic water, Cremophor EL™ (BASF, Parsippany, N.J.), and phosphate buffered saline (PBS).

Embodiments include compositions comprising an autophagy inhibitor and at least one of an NADPH modulator or a glutathione modulator, and a reactive oxygen species modulator or a pharmaceutically acceptable salt thereof. Reactive oxygen species modulators include agents that increase reactive oxygen species or inhibit reactive oxygen species detoxification. Reactive oxygen species modulators include but are not limited to 2-methoxyestradiol (2-ME). Reactive oxygen species modulators may include any compound that targets superoxide dismutases. The reactive oxygen species modulator may be combined with either of the autophagy inhibitor or the at least one of an NADPH modulator or glutathione modulator to increase effectiveness.

Embodiments include compositions comprising an autophagy inhibitor and at least one of an NADPH modulator or a glutathione modulator, and a proteasome inhibitor or a pharmaceutically acceptable salt thereof. The proteasome inhibitor may include but is not limited to MG132 and bortezomib. In an embodiment, the proteasome inhibitor is bortezomib.

An embodiment includes a composition comprising an autophgy inhibitor and at least one of an NADPH modulator or a glutathione modulator in further combination with at least one of an anti-cancer chemotherapeutic agent, a targeting agent (a targeting agent may include any one or more of the agents described for tumor targeting in example 9, below), a pharmaceutically acceptable carrier, a reactive oxygen species modulator, or a proteasome inhibitor.

An embodiment includes a composition comprising DHEA and an autophagy inhibitor.

An embodiment includes treatment of quiescent cells with an autophagy inhibitor and at least one of an NADPH modulator or a glutathione modulator. Treatment with an autophagy inhibitor may proceed, follow or be concurrent with at least one of an NADPH modulator or a glutathione modulator. The NADPH modulator or glutathione modulator may be added in combination with an autophagy inhibitor. An embodiment includes treatment with a composition comprising a combination of an autophagy inhibitor and at least one of an NADPH modulator or a glutathione modulator to induce death in quiescent cells. The composition in this method of treatment may be any one of the compositions herein. The treatment may be implemented as a method of inducing apoptotic cell death. An NADPH modulator may be any agent that reduces levels of NADPH, including a pentose phosphate pathway inhibitor, an inhibitor of the novel quiescent fibroblast NADPH production program pathway (example 21, below), and inhibitors of NADPH-generating reactions. The NADPH modulator may be but is not limited to DHEA. The inhibitor of autophagy may be but is not limited to bafilomycin. Treatment with an autophagy inhibitor and at least one of an NADPH modulator or a glutathione modulator may lead to an induction of apoptotic cell death in quiescent cells. The treatment may be carried out on targets including but not limited to any one or more of an individual cell, groups of cells, cell cultures, tumors, tissues, organs and patients to a composition herein. The treatment may include exposing any one or more of these targets to an autophagy inhibitor before, during, or after exposing the target(s) to at least one of an NADPH modulator or a glutathione modulator. A patient may be an animal. The animal may be a vertebrate. The animal may be a mammal. The animal may be a human. The patient may be a cancer patient. The composition may include a reactive oxygen species modulator. The composition may include a proteasome inhibitor. An embodiment provides treatment of quiescent cells with an autophagy inhibitor and DHEA.

In an embodiment, a method of treating cancer is provided. The method may include administering a composition comprising an autophagy inhibitor and at least one of an NADPH modulator or a glutathione modulator to a patient. The composition in this method of treatment may be any one of the compositions herein. The method may include administering an autophagy inhibitor and DHEA to a patient. A patient may be an animal. The animal may be a vertebrate. The animal may be a mammal. The animal may be a human. The patient may be a cancer patient. A method of treating cancer may include administering an autophagy inhibitor to a patient before, during or after administering at least one of an NADPH modulator or a glutathione modulator to a patient. An NADPH modulator may be any agent that reduces levels of NADPH, including a pentose phosphate pathway inhibitor, an inhibitor of the novel quiescent fibroblast NADPH production program pathway (example 21), and inhibitors of NADPH-generating reactions. The NADPH modulator may be but is not limited to DHEA. The inhibitor of autophagy may be but is not limited to bafilomycin. The composition may include a reactive oxygen species modulator. The composition may include a proteasome inhibitor. The composition may include a targeting agent. The targeting agent may be any one or more of the agents described for tumor targeting in example 9, below.

Administering may be by way of any route including but not limited to at least one of oral, injection, topical, enteral, rectal, gastrointestinal, sublingual, sublabial, buccal, epidural, intracerebral, intracerebroventricular, intracisternal, epicutaneous, intradermal, subcutaneous, nasal, intravenous, intraarterial, intramuscular, intracardiac, intraosseous, intrathecal, intraperitoneal, intravesical, intravitreal, intracavernous, intravaginal, intrauterine, extra-amniotic, transdermal, intratumoral, or transmucosal.

Any agent that is a modulator or an inhibitor as described in embodiments herein may be provided alone or in combination with at least one other agent that is also a modulator or an inhibitor as described in embodiments herein. The agent(s) may be provided with other substances, and the other substances may include but are not limited to cancer chemotheraputics. Any agent(s) used as a modulator or an inhibitor in embodiments herein, alone or with any other substance, may be provided as a pharmaceutical composition. The pharmaceutical composition may include a pharmaceutically acceptable salt or solvate. Pharmaceutically acceptable salts that may be included in embodiments herein can be found in Handbook of Pharmaceutical Salts Properties, Selection, and Use, Stahl and Wermuth (Eds.), VHCA, Verlag Helvetica Chimica Acta (Zurich, Switzerland) and WILEY-VCH (Weinheim, Federal Republic of Germany); ISBN: 3-906390-26-8, which is incorporated herein by reference as if fully set forth. The pharmaceutical composition herein may be provided with a pharmaceutically acceptable carrier, which may be selected from but is not limited to one or more in the following list: ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts or electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, talc, magnesium carbonate, kaolin, non-ionic surfactants, edible oils, physiological saline, bacteriostatic water, Cremophor EL™ (BASF, Parsippany, N.J.) and phosphate buffered saline (PBS).

An embodiment includes treatment of cancer by i) administering a method of treatment other than administering an autophagy inhibitor and at least one of an NADPH modulator or a glutathione modulator, and ii) the method of treating cancer above including administering an autophagy inhibitor and at least one of an NADPH modulator or a glutathione modulator. Step ii may include administering a composition herein. The method of treatment other than administering an NADPH modulator may be but is not limited to delivery of a chemotherapeutic agent, surgery, and delivery of radiation. For example, a method may include administering an NADPH modulator and an autophagy inhibitor, in addition to standard chemotherapy. Standard chemotherapy could include but is not limited to administration of 5-fluorouracil, cisplatin, gleevac (imatinib), or anti-angiogenic agents (bevacizumab).

An embodiment includes a method of identifying compositions that inhibit or kill quiescent cells comprising identifying a target by analyzing at least one of the metabolic flux, gene expression, protein expression, mircoRNA content, histone modification, signaling pathway activity, or physiology of quiescent cells. The method may include identifying a candidate inhibitor of the target. The candidate inhibitor may be a single agent or a combination of agents. The method may further include exposing a quiescent cell to the candidate inhibitor and identifying whether the candidate inhibitor inhibits or kills the quiescent cell. The method may further include exposing a cell culture including a quiescent cell to the candidate inhibitor and identifying whether the candidate inhibitor inhibits or kills the quiescent cell. The method may further include exposing a model organism to the candidate inhibitor and identifying whether the candidate inhibitor inhibits or kills the quiescent cell in the model organism. The model organism may be but is not limited to vertebrates. The model organism may be a mammal. The method may further include exposing a human to the candidate inhibitor and identifying whether the candidate inhibitor inhibits or kills a quiescent cell in the human.

A candidate inhibitor includes at least one agent, but may be a combination of agents. The agents may be selected from any autophagy inhibitor, NADPH modulator, glutathione modulator, proteasome inhibitor, reactive oxygen species modulator or an anti-cancer chemotherapeutic agent.

An embodiment includes a method of identifying compositions that inhibit or kill quiescent cells comprising exposing a quiescent cell to a candidate inhibitor and monitoring the physiology of the quiescent cell. The step of exposing may include administering the candidate inhibitor to a model organism and identifying whether the candidate inhibitor inhibits or kills the quiescent cell. The step of exposing may include administering the candidate inhibitor to a human and identifying whether the candidate inhibitor inhibits or kills the quiescent cell.

An embodiment includes a method of inducing apoptosis comprising exposing at least one of a cell, a cell culture, a tissue, an organ, an organism or a human to an autophagy inhibitor and the at least one of an NADPH modulator and a glutathione modulator. The autophagy inhibitor and at least one of an NADPH modulator or glutathione modulator may be administered serially, in parallel, or as part of a single composition. The composition may be any composition herein. The composition may include a reactive oxygen species modulator. The composition may include a proteasome inhibitor.

An embodiment includes a method of sensitizing quiescent cells to proteasome inhibitors comprising providing a composition comprising an NADPH modulator and an autophagy inhibitor to at least one of a cell, a cell culture, a tissue, an organ, an organism or a human. The composition may be any composition herein.

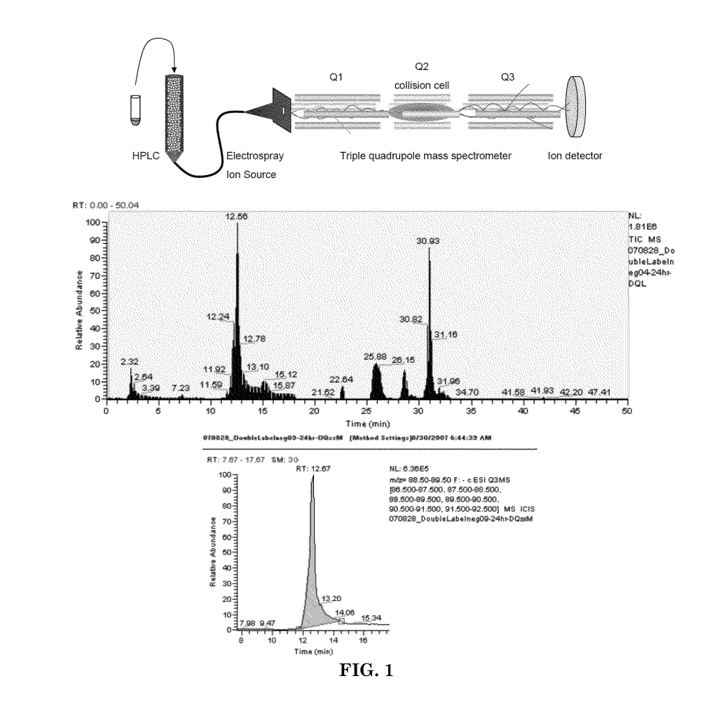

Referring to FIG. 1, a methodology for monitoring the pool size and turnover of a large number of metabolites simultaneously was developed using liquid chromatography-triple and quadrupole mass spectrometry. The methodology included technology described in Yuan J, Fowler W U, Kimball E, Lu W, Rabinowitz J D (2006) Kinetic flux profiling of nitrogen assimilation in Escherichia coli. Nat Chem Biol 2: 529-530; Munger J, Bajad S U, Coller H A, Shenk T, Rabinowitz J D (2006) Dynamics of the cellular metabolome during human cytomegalovirus infection. PLoS Pathog 2: e132; and Lu W, Kimball E, Rabinowitz JD (2006) A high-performance liquid chromatography-tandem mass spectrometry method for quantitation of nitrogen-containing intracellular metabolites. J Am Soc Mass Spectrom 17: 37-50, which are all incorporated be reference herein as if fully set forth. Metabolomic technology, flux analysis and biochemical assays were applied to investigate metabolic changes after cells enter quiescence. As described herein, it was unexpectedly discovered that certain contact-inhibited cells remained highly metabolically active while adjusting their metabolic emphasis to produce NADPH, steadily renew their proteins and lipids, and enhance secretion of specific extracellular matrix proteins.

By monitoring isotope labeling through metabolic pathways and quantitatively identifying fluxes from the data, it was shown that contact-inhibited fibroblasts utilize glucose in all branches of central carbon metabolism at rates similar to proliferating cells, with greater overflow flux from the pentose phosphate pathway (PPP) back to glycolysis. Inhibition of the PPP resulted in apoptosis preferentially in quiescent fibroblasts. By feeding the cells labeled glutamine, a “backwards” flux in the TCA cycle from α-ketoglutarate to citrate that was enhanced in contact-inhibited fibroblasts was also detected; this flux may contribute to shuttling of NADPH from the mitochondrion to cytosol for redox defense or fatty acid synthesis. The high metabolic activity of the fibroblasts was directed in part toward breakdown and re-synthesis of protein and lipid, and in part towards excretion of extracellular matrix proteins. Thus, it was unexpectedly discovered that reduced metabolic activity is not a hallmark of the quiescent state. Quiescent fibroblasts, relieved of the biosynthetic requirements associated with generating progeny, may direct their metabolic activity to preservation of self integrity and alternative functions beneficial to the organism as a whole.

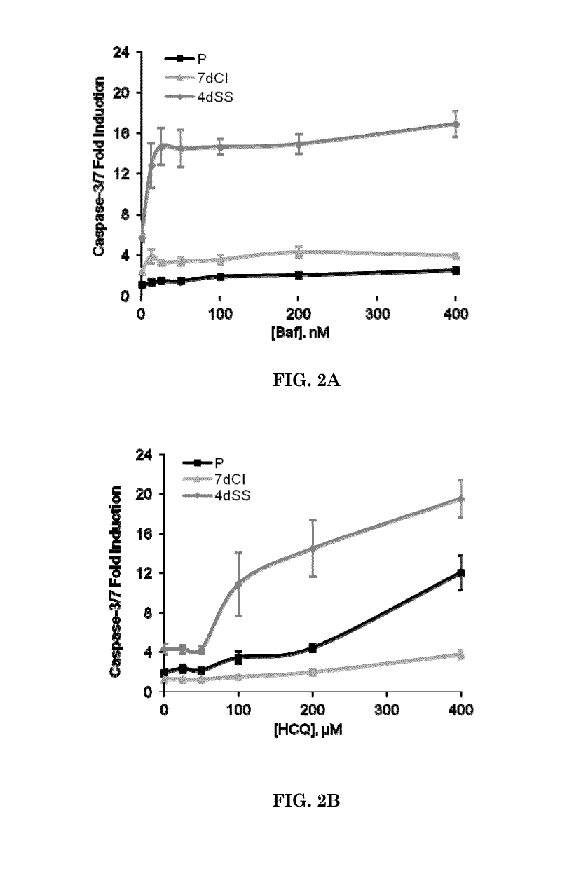

Referring to FIG. 2A, primary human fibroblasts were tested either in a proliferating state or after being induced into quiescence. Quiescence may be induced by either contact inhibition or serum starvation. Cells were treated with DHEA for four days, with bafilomycin added on the fourth day. Apoptosis was assessed based on the activity of caspase 3/7 for a fluorogenic substrate. The combination of DHEA and bafilomycin resulted in a strong (approximately 16-fold) induction of caspase activity in serum-starved fibroblasts. Referring to FIG. 2B, induction of apoptosis in serum starved fibroblasts with hydroxychloroquine is illustrated.

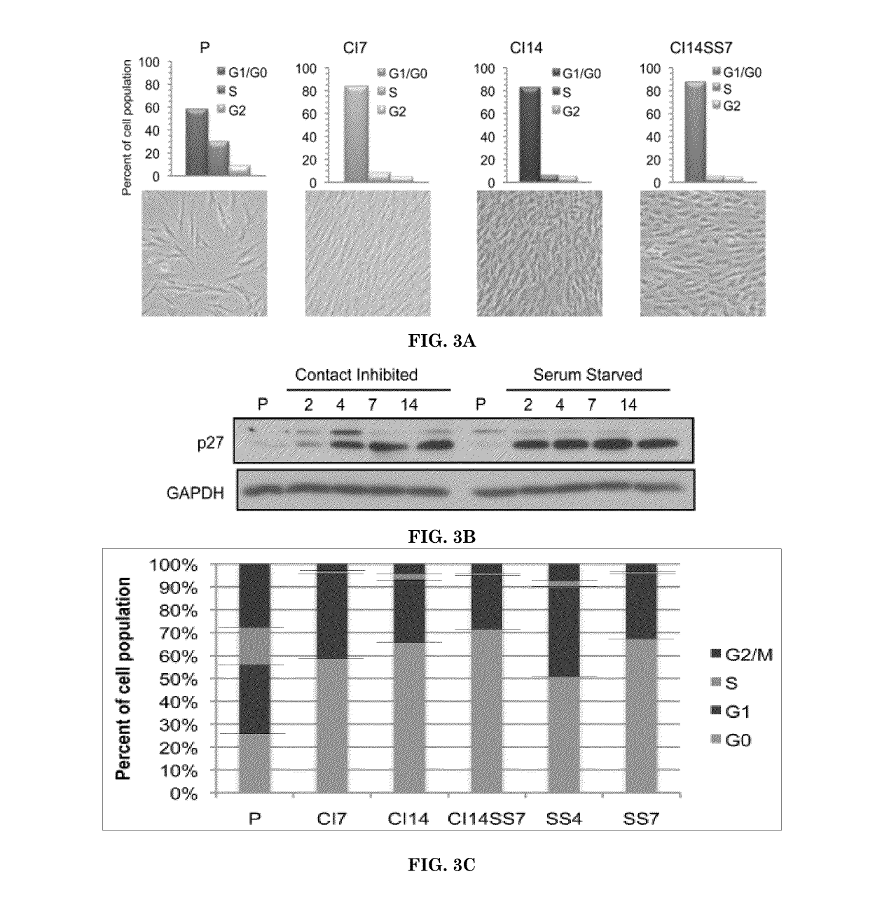

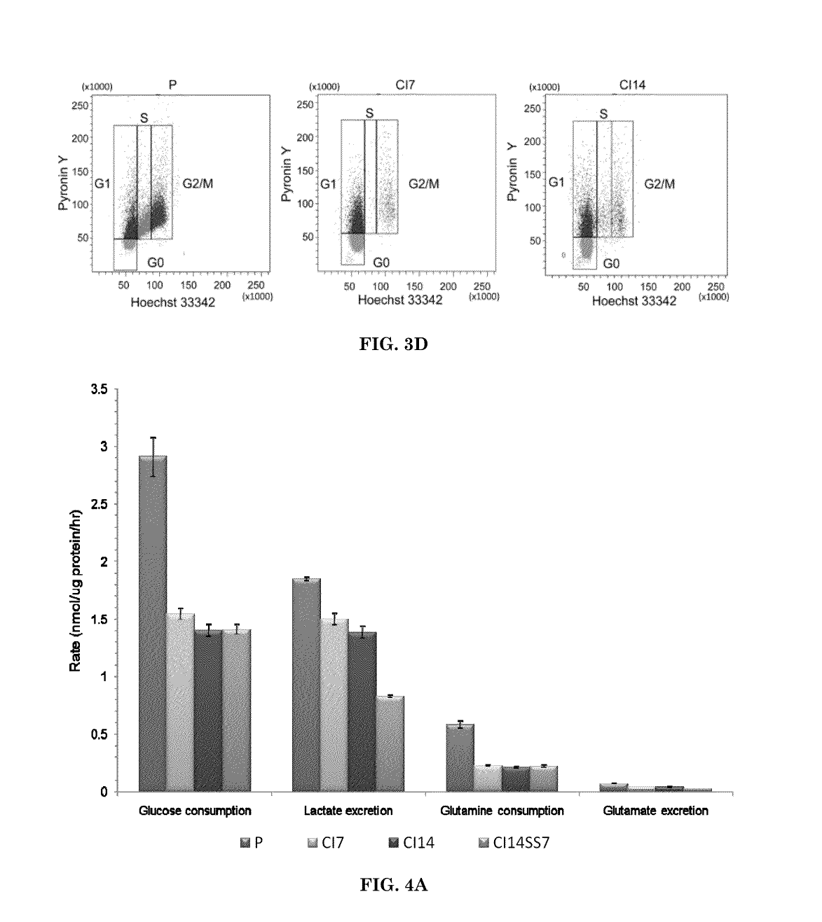

In contrast, treatment with DHEA alone resulted in 6- to 8-fold caspase induction. See FIGS. 2A, 2B and 3A. Combined treatment with DHEA and bafilomyin on proliferating fibroblats did not result in the level of induction shown in FIGS. 2A and 2B.

Often cancer treatment results are incomplete. Surviving cell populations can remain quiescent for years and eventually result in secondary tumors. The cancer stem cell theory posits that there is a small subset of the cells within a tumor that are the progenitors of the other cells. These cancer stem cells are largely quiescent, that is, not actively proliferating, but retain the capacity to proliferate and initiate a tumor in the future. Killing these quiescent tumor stem cells is challenging because most existing strategies for killing cancer cells involve killing proliferating cells, either through chemotherapy or radiation therapy. An embodiment provides a method of treating cancer comprising delivering a composition comprising a combination of a pentose phosphate pathway inhibitor and an autophagy inhibitor to a patient in need thereof. The combination may be delivered serially or in combination.

NADPH modulators. Dehydroepiandrosterone (DHEA) is a pentose phosphate pathway inhibitor, and is a potent, noncompetitive inhibitor of glucose-6-phosphate dehydrogenase. DHEA is also a naturally occurring adrenal steroid. DHEA alone has antitumor effects in animal models of spontaneous and induced tumorigenesis. DHEA may be provided as a PPP inhibitor. Other similar compounds may be provided as a PPP inhibitor. Some similar compounds are not expected to result in androgenic effects and may be provided in embodiments herein. For instance, two synthetic steroids, 16a-fluoro-5-androsten-17-one and 16a-fluoro-5a-androstan-17-one, which are likely potent inhibitors of glucose-6-phosphate dehydrogenase, are also effective in inhibiting skin papilloma development in the mouse. Another steroid, 3-b-methylandrost-5-en-17-one, is a potent antiobesity agent and also inhibits skin papilloma development. Other glucose-6-phosphate dehydrogenase inhibitors have been reported including somatostatin, a peptide of hypothalamic origin. A hypothesis is that inhibition of pentose phosphate pathway activity is via effects on NADPH levels. Any substance that affects NADPH levels may be provided in embodiments herein. The specific compounds above, analogs thereof, and similar compounds may be provided as an NADPH modulator. One or more NADPH modulator may be provided.

There are also pentose phosphate pathway inhibitors that function later in the pathway. For instance, the thiamine-utilizing enzyme transketolase functions later in the pathway and inhibition of transketolase also has anti-tumor activity. Thiamine analogs including oxythiamine have been shown to inhibit transketolase and decrease tumorigenesis. In addition, other modified forms of thiamine including non-charged thiamine analogs and prodrugs have been tested as transketolase inhibitors. See Le Huerou Y, Gunawardana I, Thomas A A, Boyd S A, de Meese J, et al. (2008) Prodrug thiamine analogs as inhibitors of the enzyme transketolase. Bioorg Med Chem Lett 18: 505-508; and Thomas A A, De Meese J, Le Huerou Y, Boyd S A, Romoff T T, et al. (2008) Non-charged thiamine analogs as inhibitors of enzyme transketolase. Bioorg Med Chem Lett 18: 509-512, which are incorporated herein by reference as if fully set forth. These transketolase inhibitors may affect rapidly proliferating cells by preventing ribose synthesis. The specific compounds above, analogs thereof, and similar compounds may be provided in a composition or method herein. One or more of these substances may be provided.

In an embodiment, the pentose phosphate pathway may be inhibited by providing siRNAs or shRNAs that target a key enzyme in the pentose phosphate pathway. Similarly, an siRNA or shRNA inhibiting a key enzyme in the novel quiescent fibroblast production pathway (example 21), NADPH producing reactions, or glutathione producing reactions may be provided. For example, an shRNA that targets the first committed step in the pentose phosphate pathway, gluocse-6-phosphate dehydrogenase, could be provided as a PPP inhibitor. An embodiment includes administering an shRNA having the sequence

[SEQ ID NO: 1]
UGCUGUUGACAGUGAGCGAGGACAACAUCGCCUGCGUUAUUAGUGAAGCC
ACAGAUGUAAUAACGCAGGCGAUGUUGUCCCUGCCUACUGCCUCGGA

as a PPP inhibitor. The specific compounds above, analogs thereof, and similar compounds may be provided as a PPP inhibitor. One or more PPP inhibitor may be provided.

Inhibition of Autophagy. In an embodiment, bafilomycin, a macrolide antibiotic, may be provided as an inhibitor of autophagy. Bafilomycin A1, or “bafilomycin” as alternatively referred to herein, is an inhibitor of vacuolar type H+-ATPase, and thereby inhibits lysosomal acidification. Concanamycin may be provided as an inhibitor of autophagy. Other compounds that have similar effects may be provided as an inhibitor of autophagy. See U.S. patent application Ser. No. 12/063,715 (Published as U.S. pre-grant publication 20080221150 and titled Prevention of Neurodegeneration by Macrolide Antibiotics), which is incorporated herein by reference as if fully set forth. The antimalarials chloroquine, hydroxychloroquine and quinacrine also inhibit lysosomal acidification and block the terminal stages of autophagic proteolysis and may be provided in embodiments herein. 3-methyladenine is an autophagy inhibitor and may be provided in embodiments herein. The specific compounds above, analogs thereof, and similar compounds may be provided as an inhibitor of the autophagy pathway. One or more inhibitor of the autophagy pathway may be provided.

A range of compounds with structural similarity to bafilomycin, chloroquine and quinacrine are available, and any one or more may be provided as an inhibitor of the autophagy pathway. In a recent study, Solomon and colleagues designed and synthesized several chloroquine analogs by introducing linear alkyl side chain and dialkyl substitution on the lateral side chain, and examined their antiproliferative effects on breast cancer cell lines. See Solomon V R, Hu C, Lee H Design and synthesis of chloroquine analogs with anti-breast cancer property. Eur J Med Chem, which is incorporated by reference herein as if fully set forth. Some of these compounds were very effective, including 7-chloro-N-(3-(4-(7-trifluoromethyl)quinolin-4-yl)piperazin-1-yl)propyl)quinolin-4-amine and {3-[4-(7-chloro-quinolin-4-yl)-piperazin-1-yl]-propyl}-(7-rifluoromethyl-quinolin-4-yl)-amine. siRNAs or shRNAs to target key proteins within the autophagy pathway may be provided as an inhibitor of the autophagy pathway. For example, siRNAs or shRNAs targeting expression of at least one of atg5, atg7, lc3, atg8 or beclin1 may be provided. One sequence targeting expression of atg5 is

[SEQ ID NO: 2]
GUGAGAUAUGGUUUGAAUAdTdT(sense)
[SEQ ID NO: 3]
UAUUCAAACCAUAUCUCACdTdT(anti-sense).

Another sequence targeting expression of atg5 is

[SEQ ID NO: 4]
GAUAUGGUUUGAAUAUGAAdTdT (sense)
[SEQ ID NO: 5]
UUCAUAUUCAAACCAUAUCdTdT (anti-sense).

Another sequence targeting expression of atg5 is
TRCN0000151963 (shRNA, Position 1170 (3′-UTR) of human atg5), Target Sequence:

[SEQ ID NO: 6]
CCTGAACAGAATCATCCTTAA;

### Hairpin Sequence:

[SEQ ID NO: 7]
5′-GCCGG-CCTGAACAGAATCATCCITAA-CTCGAG-TTAAGGATGATT
CTGTTCAGG-TTTTTTG-3′

Another sequence targeting expression of atg5 is TRCN00003330394 (sbRNA, Position 1197 (3′UTR) of human atg5), Target Sequence:

[SEQ ID NO: 8]
CCTGAACAGAATCATCCTTAA;

### Hairpin Sequence:

[SEQ ID NO: 9]
5′-CCGG-CCTGAACAGAATCATCCTTAA-CTCGAG-TTAAGGATGATTC
TGTTCAGG-TTTTG-3′

One sequence targeting expression of atg7 is

[SEQ ID NO: 10]
CAGUUACAGAUGGAGCUAAdTdT(sense)
[SEQ ID NO: 11]
UUAGCUCCAUCUGUAACUGdTdT (anti-sense).

Another sequence targeting expression of atg7 is

[SEQ ID NO: 12]
GAGAUAUGGGAAUCCAUAAdTdT (sense)
[SEQ ID NO: 13]
UUAUGGAUUCCCAUAUCUCdTdT (anti-sense).

Another sequence targeting atg7 is

[SEQ ID NO: 14]
CAGCUAUUGGAACACUGUAdTdT (sense)
[SEQ ID NO: 15]
UACAGUGUUCCAAUAGCUGdTdT (anti-sense).

Another sequence targeting atg7 is
TRCN0000007 584 (shRNA, Position 2173 (3′-UTR) of human atg7), Target Sequence:

[SEQ ID NO: 16]
GCCTGCTGAGGAGCTCTCCAT

### Hairpin Sequence:

[SEQ ID NO: 17]
5′-CCGG-GCCTGCTGAGGAGCTCTCCAT-CTCGAG-
ATGGAGAGCTCCTCAGCAGGC-TTTTT-3′

Another sequence targeting atg7 is
TRCN0000007587 (shRNA, Position 268 (CDS) of human atg7), Target Sequence:

[SEQ ID NO: 18]
CCCAGCTATTGGAACACTGTA

### Hairpin Sequence:

[SEQ ID NO: 19]
5′CCGG-CCCAGCTATTGGAACACTGTA-CTCGAG-TACAGTGTTCCAAT
AGCTGGG-TTTTT-3′

One sequence targeting expression of beclin1 is

[SEQ ID NO: 20]
CAGAAGGCUCGAGAAGGUAUAUUGCUGUUGACAGUGAGCGAGACAGUUU
GGCACAAUCAAUAUAGUGAAGCCACAGAUGUAUAUUGAUUGUGCCAAAC
UGUCCUGCCUACUGCCUCGGAAUUCAAGGGGCUACUUUAG.

This sequence may be referred to as MSCV/LMP-shBeclin1#8.
Another sequence targeting expression of beclin1 is

[SEQ ID NO: 21]
GUUUGGAGAUCUUAGAGCAdTdT (Sense)
[SEQ ID NO: 22]
UGCUCUAAGAUCUCCAAACdTdT (Antisense).

Other sequences targeting atg5, atg7, lc3, atg8, and beclin1 may be available from vendors; for example, Sigma Aldrich. The sequences of atg5 (NM_{—}004849), atg7 (NM_{—}006395) and beclin1 (NM_{—}003766) are provided below. An siRNA or shRNA targeting expression of any gene involved in autophagy may be designed based on the gene sequence and general knowledge of siRNA or shRNA. An siRNA or shRNA targeting expression of atg5, atg7 or beclin1 may be designed based on the gene sequence (e.g., NM_{—}004849, NM_{—}006395 or NM_{—}003766, below) and general knowledge of siRNA or shRNA. The specific compounds above, analogs thereof, and similar compounds may be provided as an inhibitor of the autophagy pathway. One or more inhibitor of the autophagy pathway may be provided.

In embodiments where a combination of agents are provided, they may be delivered or administered in any fashion, including but not limited to being delivered or administered together, serially, or in parallel with one another through different delivery or administration events.

An embodiment includes methods of inhibiting at least one pathway involved in quiescent cell survival or maintenance including administering at least one substance that inhibits at least one pathway to a cell, model organism, or human. An embodiment includes one or more substances that inhibit at least one pathway involved in quiescent cell survival or maintenance. An inhibitor of at least one pathway involved in quiescent cell survival or maintenance may be but is not limited to a PPP inhibitor, an autophagy inhibitor, or a combination of a PPP inhibitor and an autophagy inhibitor.

The data herein suggests three avenues for energy utilization in quiescent cells. First, contact-inhibited fibroblasts may continuously degrade and resynthesize their macromolecules and membrane components via increased autophagy, a strategy that would help to ensure that old and potentially damaged macromolecules and membranes do not accumulate. The data herein also suggest that contact-inhibited fibroblasts may degrade protein and fatty acids at an enhanced rate compared with proliferating fibroblasts. A conclusion consistent with the data is that the proliferating and contact-inhibited fibroblasts synthesize amino acids and fatty acids at rates that are comparable, with the new biomass contributing to new cells in proliferating fibroblasts and replacing degraded molecules in the contact-inhibited fibroblasts.

Second, contact-inhibited and serum-starved fibroblasts induce pathways that generate NADPH. As described herein, three NADPH generating enzymes, G6PD, PGD and IDH1, are induced in quiescent compared with proliferating fibroblasts. The results suggest that quiescent fibroblasts activate an NADPH-generating program of enzyme induction. One role of the NADPH may be to ensure the availability of reduced glutathione and thioredoxin for the detoxification of free radicals. Another role for the NADPH generated may be to support re-synthesis of fatty acids, as fatty acid degradation yields NADH while synthesis requires NADPH.

The discoveries herein suggest that contact-inhibited and serum-starved fibroblasts are particularly susceptible to apoptosis induced by treatment with DHEA, a pentose phosphate pathway inhibitor. The ability to selectively kill quiescent cells has therapeutic potential. For instance, tumor stem cells may exist in a quiescent state for years, while retaining the capacity to emerge from dormancy, proliferate and initiate a tumor recurrence. Embodiments herein provide compositions that target the pathways invoked by these cells to facilitate their survival during dormancy and could be useful additions to a therapeutic arsenal. Embodiments also include methods of treating cancer by administering any chemical, biological and/or physical agent that inhibits these pathways. It was discovered that contact-inhibited and serum-starved fibroblasts rely on the PPP and possibly other NADPH-generating reactions for viability. Embodiments herein provide one or more small molecule inhibitors for targeting quiescent tumor cells. Embodiments herein provide methods of treating cancer comprising administering small molecule inhibitors of the PPP to a patient in need thereof. The small molecule inhibitors may be useful for targeting quiescent tumor cells.

Embodiments herein include methods of screening for inhibitors of pathways involved in quiescent cell survival or maintenance comprising providing a candidate inhibitor and measuring or monitoring at least one of i) metabolic flux through the PPP, ii) metabolic flux through the novel quiescent fibroblast NADPH production program pathway, iii) apoptosis, iv) autophagy, v) cell death or necrotic cell death, vi) effects on the cell cycle of cells entering or exiting quiescence, and vii) effects on the gene expression patterns of quiescent cells. Embodiments herein include methods of screening for cancer therapeutic agents comprising exposing a quiescent cell to a candidate inhibitor and measuring or monitoring at least one of i) metabolic flux through the PPP, ii) metablolic flux through the novel quiescent fibroblast NADPH production program pathway iii) apoptosis, iv) autophagy, v) cell death or necrotic cell death, vi) effects on the cell cycle of cells entering or exiting quiescence, and vii) effects on the gene expression patterns of quiescent cells. Embodiments herein include a method of screening candidate agents for the ability to inhibit or kill quiescent cells including i) exposing a quiescent cell line to at least one candidate agent, and ii) assessing quiescent cell survival, morphology, or physiological state in response to the at least one candidate agent. The method may include making or providing the quiescent cell line. The quiescent cell or quiescent cell line used in the embodiments herein may be but are not limited to those isolated or derived from i) quiescent dermal human fibroblasts, ii) quiescent human fibroblasts from other sources, iii) quiescent mouse embryo fibroblasts, iv) primary resting lymphocytes, v) stellate liver cells, vi) keratinocytes, vii) hematopoietic stem cells, and vii) cancer stem cells from cancer cell lines.

Further embodiments herein may be formed by supplementing an embodiment with one or more element from any one or more other embodiment herein, and/or substituting one or more element from one embodiment with one or more element from one or more other embodiment herein.

### EXAMPLES

The following non-limiting examples are provided to illustrate particular embodiments. The embodiments throughout may be supplemented with one or more detail from one or more example below, and/or one or more element from an embodiment may be substituted with one or more detail from one or more example below.

Examples showing pentose phosphate pathway (or PPP) inhibition or the novel quiescent fibroblast NADPH production pathway indicate agents that may be NADPH modulators and methods to modulate NADPH.

### Example 1

Experimentation on normal cells from mice and humans. A series of experiments could be done to determine whether a combination of an NADPH modulator and autophagy inhibition (combination treatment) results in killing of quiescent tumor cells and consequently anti-tumorigenic effects. Initially, it could be determined whether this combination treatment results in death of quiescent cells in other normal cells. Any NADPH modulator and any autophagy inhibitor could be tested by these experiments.

Human fibroblasts from various anatomical sites may be tested to determine whether these fibroblasts apoptose in response to a combination of NADPH reduction and autophagy inhibition. The fibroblasts in these tests may be induced to quiescence. The fibroblasts in these tests may be induced to quiescence by serum-starvation.

B-lymphocytes may be isolated from spleens; e.g., mouse spleens. These cells can be cultured and stimulated to divide in vitro. The resting lymphocytes and the stimulated lymphocytes could be monitored to determine the extent of apoptosis in response to the combination treatment (treatment with an NADPH modulator and an inhibitor of the autophagy pathway) and to determine whether quiescent lymphocytes are more susceptible to combination treatment.

Long-term hematopoietic stem cells may also be isolated; e.g., from mouse bone marrow. These quiescent stem cells can be compared with proliferative myeloid progenitor cells. A protocol for isolation of long-term hematoepoietic stem cells and myeloid progenitor cells from mouse bone marrow based on FACS sorting for multiple markers sequentially has been described in Passegue E, Wagers A J, Giuriato S, Anderson W C, Weissman I L (2005) Global analysis of proliferation and cell cycle gene expression in the regulation of hematopoietic stem and progenitor cell fates. J Exp Med 202: 1599-1611, which is incorporated herein by reference as if fully set forth. Cells can be cultured and monitored with respect to their apoptotic response to treatment with pentose phosphate pathway and autophagy inhibitors in combination.

### Example 2

Cancer stem cells in vitro studies. Within cancer cell populations, there exists a subpopulation that has characteristics of cancer stem cells. These cancer stem cell-like cells can be identified as a “side population” within the cancer cell population based on their low intensity staining with certain dyes (for example, see Sun G, et al. Identification of stem-like cells in head and neck cancer cell lines. Identification of stem-like cells in head and neck cancer cell lines. Anticancer Res 30: 2005-2010, which is incorporated herein by reference as if fully set forth). The cancer stem cell-like subpopulation could be sorted out from cancer cell lines and used to determine whether the stem cell-like population exhibits more apoptosis from a treatment or combination treatment described herein than the bulk population.

Tumors may be collected, used to form a single cell suspension, and the cancer stem cell-like cells could be sorted out. Tumors from any source may be used; e.g., human, mouse, etc. for experiments or methods described herein.

### Example 3

Mouse models of cancer: Transplanted tumors. Several mouse models may be used to test the efficacy of a treatment or combination treatment on transplanted tumor cells, chemically-induced tumors and spontaneous tumors. The following dosing schemes are exemplary and may be revised based on results of experimentation. In a previous study, both oxythiamine and DHEA were shown to inhibit tumor growth in a model involving intraperitoneal injection of tumor cells. See Boros L G, Puigjaner J, Cascante M, Lee W N, Brandes J L, et al. (1997) Oxythiamine and dehydroepiandrosterone inhibit the nonoxidative synthesis of ribose and tumor cell proliferation. Cancer Res 57: 4242-4248, which is incorporated herein by reference as if fully set forth. Experiments regarding the embodiments herein could be performed with this model because of its simplicity. NADPH modulators and autophagy inhibitors identified as promising in the experiments described above could be utilized in such a study. Approximately 16, 8 week old C57/B16 mice could be tested. Ehrlich's ascites tumor cells could be harvested from a continuously growing cell population hosted by a host animal. Tumor cells could be normalized for cell number and implanted. Animals could be injected with 0.2 ml of suspension (2×10⁴ cells) i.p. Tumor volume, average cell volume and cell viability could be measured on day 8 after days incubation and 3 days drug treatment. Mice could be divided into vehicle control, pentose phosphate pathway inhibitor only, autophagy inhibitor only and both pentose phosphate pathway and autophagy inhibitor groups. The exact dosing and compounds used could change based on preliminary cell culture experiments. An example of dosing may be 60 mg/ml solutions of DHEA or hydroxychloroquine prepared in a 1% DMSO-saline mixture and 0.2 ml (400 mg/kg) of each drug could be injected i.p. for 3 days. Control animals could receive 0.2 ml of 1% DMSO-saline i.p. injections daily. Differences between the treated and control groups in tumor growth rates will be analyzed with student's t-tests.

### Example 4

GIST xenograft model. A recent study reported the efficacy of autophagy inhibition for gastrointestinal stromal tumors (GIST). See Gupta A, Roy S, Lazar A J, Wang W L, McAuliffe J C, et al. (2010) Autophagy inhibition and antimalarials promote cell death in gastrointestinal stromal tumor (GIST). Proc Natl Acad Sci USA 107(32):14333-8, which is incorporated herein by reference as if fully set forth. GIST is the most common mesenchymal neoplasm of the gastrointestinal tract. Most GISTs contain activating KIT or PDGF receptor mutations. Treatment with imatinib mesylate, a small molecule tyrosine kinase inhibitor is highly effective, but still the quiescent cells often remain, and these cells can give rise to recurrent disease. A similar GIST xenograft model may be utilized where the treatment includes standard GIST treatment and treatment with an autophagy inhibitor and PPP inhibitor. For example, the treatment may include administering imatinib, chloroquine/quinacrine and a pentose phosphate pathway inhibitor.

Chemically induced tumors in mice. Development of skin papillomas and carcinomas by topical treatment with 7,12-dimethylbenz(a)anthracene (DMBA) is reduced by DHEA. An even more potent chemopreventive effect was achieved by another steroid that is a potent antiobesity and antidiabetic agent; 3-β-methylandrost-5-en-17-one. See Pashko L L, Hard G C, Rovito R J, Williams J R, Sobel E L, et al. (1985) Inhibition of 7,12-dimethylbenz(a)anthracene-induced skin papillomas and carcinomas by dehydroepiandrosterone and 3-beta-methylandrost-5-en-17-one in mice. Cancer Res 45: 164-166, which is incorporated herein by reference as if fully set forth. This model system may be utilized to monitor the effects of a combination of pentose phosphate pathway and autophagy inhibition. Female CD1 mice could be shaved at 6 or 7 weeks of age. Three days later, a dose of 200 nmol of DMBA could be applied. Beginning 2 weeks later, 100 nmol of DMBA in 0.2 ml acetone could be applied once weekly. NADPH reduction agents and/or autophagy pathway inhibitors could be applied at a dose of 100 μg PPP inhibitor (e.g., DHEA) and 400 μg autophagy inhibitor (e.g., hydroxychloroquine) in 0.2 ml acetone for 1 hr before each weekly application of DMBA. Mice could be palpated for tumors weekly for a year and the total number of papillomas and suspected carcinomas could be recorded.

### Example 5

Colon cancer model. DHEA has been found to have a chemopreventative effect on colon cancer. See Osawa E, Nakajima A, Yoshida S, Omura M, Nagase H, et al. (2002) Chemoprevention of precursors to colon cancer by dehydroepiandrosterone (DHEA). Life Sci 70: 2623-2630, which is incorporated herein as if fully set forth. A combination of NADPH reduction and autophagy inhibition may be tested in this model as well. Eight week old BALB/c mice could be administered an NADPH modulator (e.g., DHEA (0.8% w/w)), an autophagy inhibitor (e.g., hydroxychloroquine (0.8% w/w), both or neither. Compounds could be administered to the mice for five weeks both during and after carcinogen administration. After one week's aclimatization at the housing environment and basal diet, mice could be injected with azoxymethane 10 mg/kg intraperitoneally, twice, with a one week interval. Mice could be sacrificed three weeks after the second i.p. injection of AOM. The entire colon could be removed and fixed and the number of aberrant crypt foci could be determined based on their distinction from normal crypts, their larger size, increased pericryptal area, greater staining intensity, elevation above the adjacent normal crypts and abnormally shaped lumina.

### Example 6

Multiple organs. Among F344 rats treated with dihydroxy-di-n-propylnitrosamine (DHPN), those that were subsequently exposed to DHEA exhibited decreased development of thyroid tumors. See Moore M A, Thamavit W, Tsuda H, Sato K, Ichihara A, et al. (1986) Modifying influence of dehydroepiandrosterone on the development of dihydroxy-di-n-propylnitrosamine-initiated lesions in the thyroid, lung and liver of F344 rats. Carcinogenesis 7: 311-316, which is incorporated herein by reference as if fully set forth. In this model, however, DHEA treatment was also associated with development of basophilic hepatocellular foci. F344 rats could be assigned to groups: DHEA (or other pentose phosphate pathway inhibitor), hydroxychloroquine (or other autophagy inhibitor), both or neither. Rats could receive a single 1000 mg/kg body weight dose of DHPN by i.p. injection followed by a further three injections once every two weeks of 250 mg/kg starting 3 weeks later. After week 8, the experimental animals could be maintained on a basal diet, a DHEA diet (0.6% w/w), hydroxychloraquine diet (0.6% w/w) or a diet containing both. Animals could be maintained on an appropriate diet until sacrifice, half at week and the other half at week 32. Upon sacrifice, the major organs could be removed and portions fixed. Preneoplastic foci and tumors could be identified and counted in the lung, thyroid, urinary bladder, and liver.

### Example 7

Spontaneous tumor formation. Spontaneous cancer models could also be tested. Long-term DHEA treatment has been shown to inhibit spontaneous breast cancer occurrence in female C3H (A^{vy}/a) mice. See Schwartz A G (1979) Inhibition of spontaneous breast cancer formation in female C3H(A^{vy}/a) mice by long-term treatment with dehydroepiandrosterone. Cancer Res 39: 1129-1132, which is incorporated herein by reference as if fully set forth. A similar experiment may be performed with both an NADPH modulator and an inhibitor of autophagy. Breeding pairs of C3H mice could be crossed with female C3H (a/a) mice that carry the mammary tumor virus. Females with the mammary tumor virus could be divided into groups. An example of the dosing scheme would be that one group could receive 450 mg of DHEA per kg (suspension in sesame oil) by p.o. intubation 3 times weekly, another could receive only sesame oil, a third could receive 6 mg/kg hydroxychloroquine i.p., and a fourth could receive DHEA and hydroxychloroquine. Breast cancer incidence could be monitored.

Another model of spontaneous tumor formation in mice is the p53-deficient mouse. DHEA and a DHEA analog 16α-fluoro-5-androsten-17-one have been shown to delay death due to neoplasms, largely by suppressing lymphoblastic lymphoma in this model. See Perkins S N, Hursting S D, Haines D C, James S J, Miller B J, et al. (1997) Chemoprevention of spontaneous tumorigenesis in nullizygous p53-deficient mice by dehydroepiandrosterone and its analog 16alpha-fluoro-5-androsten-17-one. Carcinogenesis 18: 989-994, which is incorporated herein by reference as if fully set forth. As an example, DHEA, hydroxychloroquine, both or neither could be administered to p53 knockout mice. DHEA could be added to the diet at 0.3% (w/w) and hydroxychloroquine could be added to the diet at 6 mg/kg. Tumor development could be monitored by autopsy of dead mice and effects of the inhibitors individually and in combination may be determined.

### Example 8

Treating human tumors. Existing protocols provide an example of the types of studies that could be performed. One current clinical trial involves autophagy and anti-angiongenesis in colorectal carcinoma testing hydroxychloroquine and an angiongenesis inhibitor bevacizumab. This study could be expanded to include both an autophagy inhibitor and an NADPH modulator. Sandard chemotherapy would be given to all patients, and would involve oxaliplatin given by vein and capecitabine (oral 5-fluorouracil) by pill. In this study, bevacizumab would be given by vein. Hydroxychloroquine and a pentose phosphate pathway inhibitor would be given intravenously or by pill as well. For hydroxychloroquine, 200 mg taken three times a day orally would be an example of a dosing course. Endpoints would include time to progression, percent one-year survival and overall survival. Overall toxicity would be determined. And patient specimens would be collected to assess the effects of hydroxychloroquine on autophagy in the patients.

Hydroxychloroquine is also being tested for its effects in metastatic hormone refractory protstate cancer. Patients with metastatic prostate cancer with progression after initial hormonal therapy will be studied. Patients will be given docetaxel and either an autophagy inhibitor and a pentose phosphate pathway inhibitor, or only docetaxel and outcomes will be monitored as described above. Non-small cell lung cancer would also be tested. A standard care for lung cancer which consists of chemotherapy drugs, paclitaxel and carboplatin plus bevacizumab to target blood vessels could be provided. Also, the addition of an autophagy inhibitor and an NADPH modulator may be tested to determine if the addition improves outcome. For metastatic breast cancer, standard care of ixabepilone would be provided alone or with an autophagy inhibitor and an NADPH modulator.

As another example, a pilot study has been performed to monitor DHEA activity in cervical cancer in 12 women with low-grade dysplasia, confirmed by colposcopic exam. See Suh-Burgmann E, Sivret J, Duska L R, Del Carmen M, Seiden M V (2003) Long-term administration of intravaginal dehydroepiandrosterone on regression of low-grade cervical dysplasia—a pilot study. Gynecol Obstet Invest 55: 25-31, which is incorporated herein by reference as if fully set forth. The study concluded that DHEA is safe to administer and that it may promote regression of low-grade cervical lesions. A similar study may be performed using a PPP inhibitor (e.g., DHEA), and an autophagy inhibitor (e.g., hydroxychloroquine) in the formulation. Women with low-grade dysplasia could be enrolled. The women could be given 150 mg of intravaginal micronized DHEA alone, micronized hydroxychloroquine daily, both, or vehicle control for up to 6 months. Follow-up evaluations of the cervix could be performed at 3 months and 6 months. Serum levels of DHEA, androstenedione, testosterone, and hydroxychloroquine could be tested. The number of women with normal colposcopic exams and the number with atypical cells could be determined and the effects of the compounds individually and together could be assessed.

### Example 9

Targeting agents and tumor targeting. In addition to the methods of application described here, tumor targeting approaches may be provided that may allow treatment with NADPH modulators and autophagy inhibitors directed to tumor cells. Multiple tumor targeting strategies are emerging, several of which could be used to deliver small molecule or siRNA derived inhibitors of NADPH production and autophagy pathway to tumor cells. One approach employs non-pathogenic obligate anaerobic bacteria for targeting tumors. These bacteria could home to tumors because of their low oxygen environment. See Taniguchi S, Fujimori M, Sasaki T, Tsutsui H, Shimatani Y, et al. Targeting solid tumors with non-pathogenic obligate anaerobic bacteria. Cancer Sci., which is incorporated herein by reference as if fully set forth. As one example, the non-pathogenic obligate anaerobic bacterium Bifidobacterium longum is being explored as a vehicle to selectively recognize and target the anaerobic conditions in solid cancer tissues that result from low oxygen pressure inside tumor masses. The bacteria can colonize and destroy solid tumors themselves. The bacteria can also be genetically engineered to overexpress a particular protein and express it at the tumor site. This approach can be utilized to specifically inhibit the NADPH production and autophagy within tumor tissue. As one example, Clostridia have been genetically engineered to express genes for pro-drug converting enzymes. See Ryan R M, Green J, Lewis C E (2006) Use of bacteria in anti-cancer therapies. Bioessays 28: 84-94, which is incorporated herein by reference as if fully set forth. DHEA or a derivative could be introduced systemically in an inactive form, and bacteria expressing a specific enzyme that activates the pre-DHEA could be targeted to the tumor. Bafilomycin could also potentially be targeted to tumors through a similar mechanism. Alternatively, shRNAs to the NADPH production reactions e.g. the pentose phosphate pathway or the novel quiescent fibroblast NADPH production pathway and the autophagy pathway might be deliverable through bacteria as vectors. Similar targeting schemes could be utilized for any NADPH modulator and/or any autophagy inhibitor.

Metallic nanoparticles are also being investigated as a new method for specifically targeting tumor tissue. See Ahmad M Z, Akhter S, Jain G K, Rahman M, Pathan S A, et al. (2010) Metallic nanoparticles: technology overview & drug delivery applications in oncology. Expert Opin Drug Deliv 7: 927-942, which is incorporated herein as if fully set forth. A recent report described a cancer-cell specific magnetic nanovector construct for efficient siRNA delivery and non-invasive monitoring through MRI. See Veiseh O, Kievit F M, Fang C, Mu N, Jana S, et al. (2010) Chlorotoxin bound magnetic nanovector tailored for cancer cell targeting, imaging, and siRNA delivery. Biomaterials 31(31): 8032-8042, which is incorporated herein by reference as if fully set forth. The base of the nanovector construct is superparamagnetic iron oxide nanoparticle core coated with polyethylene glycol-grafted chitosan and polyethylenimine. The vector is designed to deliver siRNAs and uses a tumor-targeting peptide chlorotoxin. Such a delivery system could also deliver agents, including NADPH modulators or autophagy inhibitors to sites including but not limited to cells, tissues, organs and tumors.

As another example, near infrared fluorescent small molecules and nanoparticles have been designed to specifically target integrin molecules present in tumor vasculature. See Akers W J, Zhang Z, Berezin M, Ye Y, Agee A, et al. (2010) Targeting of alpha(nu)beta(3)-integrins expressed on tumor tissue and neovasculature using fluorescent small molecules and nanoparticles. Nanomedicine (Lond) 5: 715-726, which is incorporated herein by reference as if fully set forth. In a similar example, an αᵥβ₃-specific nanoprobe of fluorescent superparamagnetic polymeric micelles were produced. See Talelli M, Iman M, Varkouhi A K, Rijcken C J, Schiffelers R M, et al. (2010) Core-crosslinked polymeric micelles with controlled release of covalently entrapped doxorubicin. Biomaterials 31: 7797-7804, which is incorporated by reference herein as if fully set forth. The micelles were encoded with an αᵥβ₃-specific peptide and observed to accumulate in human lung cancer subcutaneous tumor xenografts. Systems as set forth above, or any other targeting method, could be exploited to deliver pentose phosphate pathway and/or autophagy inhibitors to tumors.

### Example 10

A model for cellular quiescence in primary fibroblasts. In some examples herein, newborn dermal fibroblasts are utilized as a model system of quiescence. Model systems can be found in Coller H A, Sang L, Roberts J M (2006) A new description of cellular quiescence. PLoS Biol 4: e83; Sang L, Coller H A, Roberts J M (2008) Control of the reversibility of cellular quiescence by the transcriptional repressor HES1. Science 321: 1095-1100; and Pollina E A, Legesse-Miller A, Haley E M, Goodpaster T, Randolph-Habecker J, et al. (2008) Regulating the angiogenic balance in tissues. Cell Cycle 7: 2056-2070, which are incorporated herein by reference as if fully set forth. In vitro, primary fibroblasts isolated directly from newborn foreskin can be induced into reversible quiescence by serum withdrawal or contact inhibition. Unlike most primary cells, fibroblasts remain healthy in culture in a quiescent state for as long as thirty days with little apoptosis or senescence, and can then re-enter the cell cycle. In vivo, quiescent fibroblasts are central to normal physiology as the major players in the synthesis of extracellular matrix necessary for the formation of cellular tissues. In response to a wound, fibroblasts enter the cell cycle from quiescence, proliferate and secrete a collagen-rich extracellular matrix, pro-angiogenesis factors that recruit new blood vessels, and other molecules that facilitate the wound healing response. Scarring and fibrosis result from excessive fibroblast proliferation and secretion of extracellular matrix during and after wound healing.

A model system that allows monitoring metabolic differences between proliferating and quiescent cells was developed. Primary dermal fibroblasts were expanded and analyzed while actively proliferating (P), after one week of growth to confluence (contact inhibition for 7 days, CI7), after two weeks of confluence (contact inhibition for 14 days, CI14), or after two weeks of confluence with serum concentrations decreased for the final week from 10% to 0.1% (CI14SS7). Alternatively, fibroblasts were plated sparsely so that they did not touch each other and induced into quiescence by serum starvation and monitored after four days (SS4) or seven days (SS7). In quiescent fibroblasts, the fraction of cells with 2N DNA content increased so that 80% or more of the cells were in the G0/G1 phase of the cell cycle (FIG. 3A). The fraction of cells in S phase was significantly reduced, indicating that very few cells were actively dividing under these conditions. In both contact-inhibited and serum-starved fibroblasts, levels of the cyclin-dependent kinase inhibitor p27^{Kip1} were upregulated, as expected for cells that entered quiescence (FIG. 3B). In addition, staining with pyronin Y for total RNA indicated that the fraction of cells with low pyronin Y, interpreted as cells in G0, increased in fibroblasts induced into quiescence by all of these methods (FIG. 3C). Pyronin Y labeling data indicate that in the contact-inhibited and serum-starved cell populations investigated as quiescence models, approximately 60-75% of the cells are in G0 and most of the remainder are in G1.

### Example 11

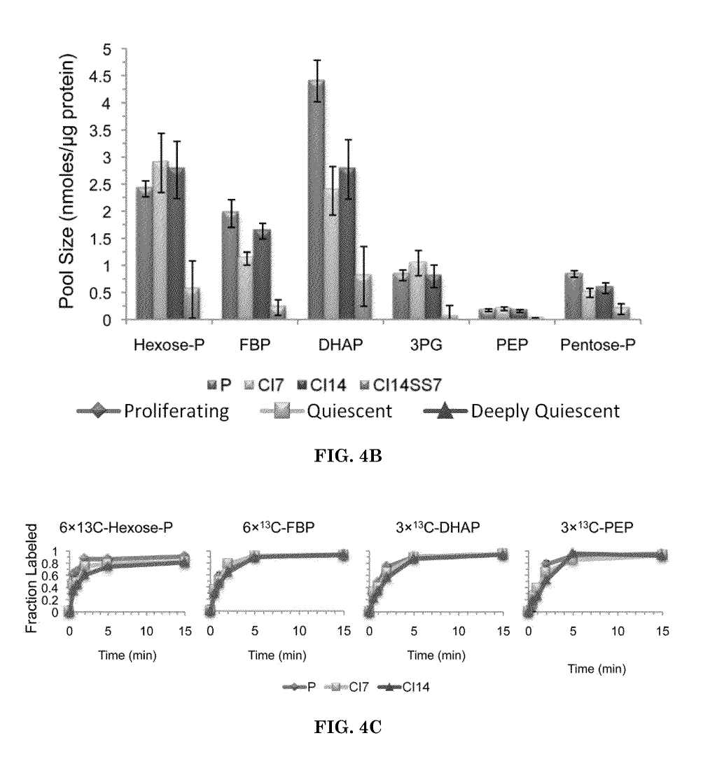

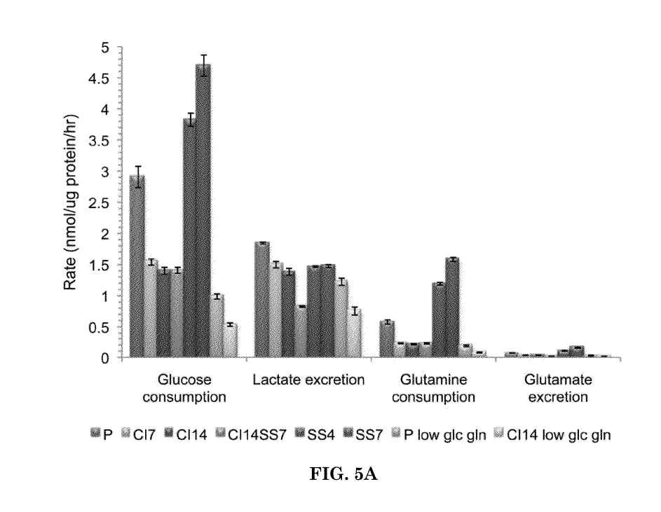

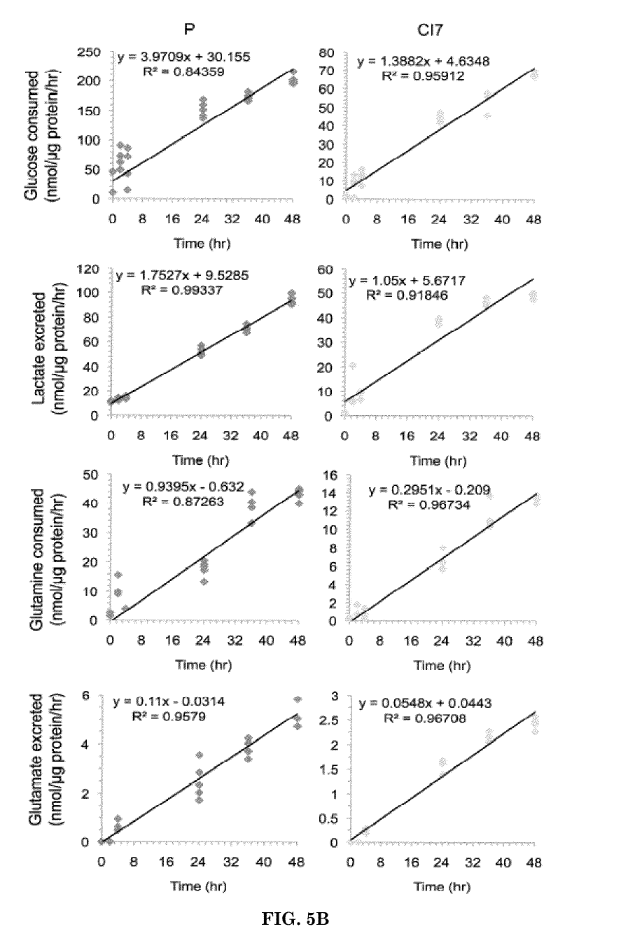

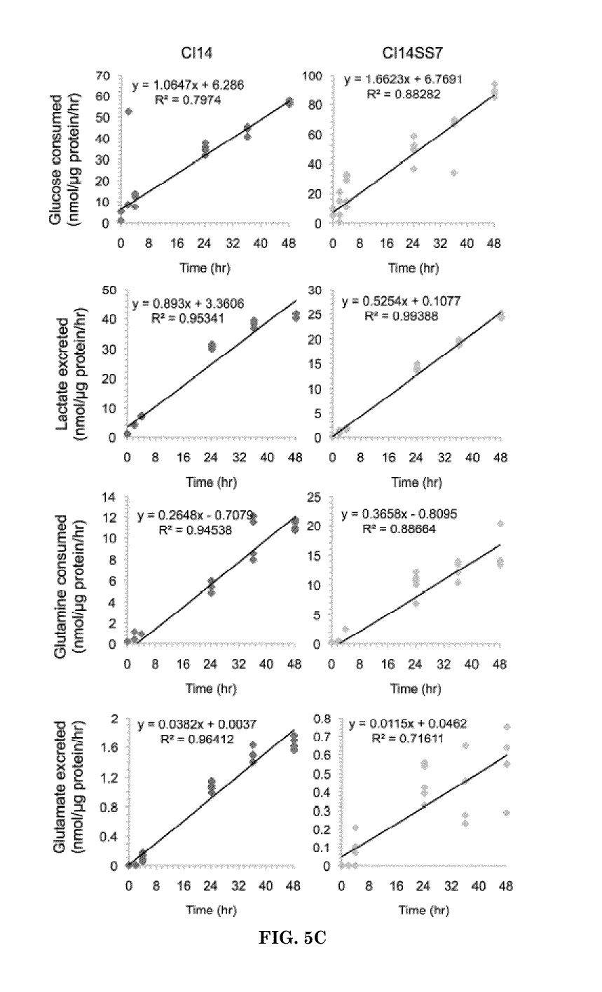

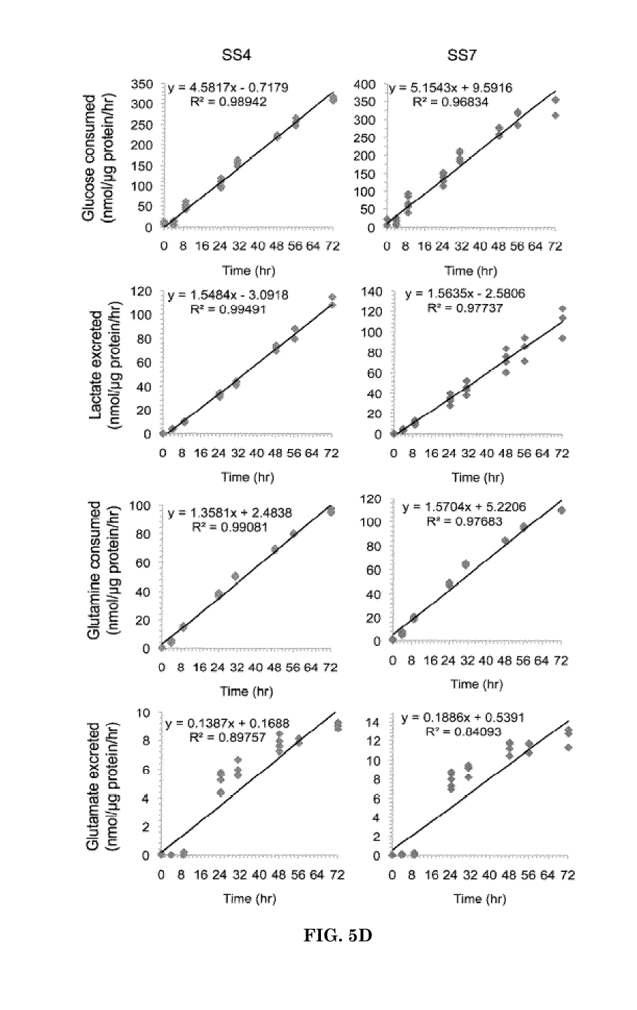

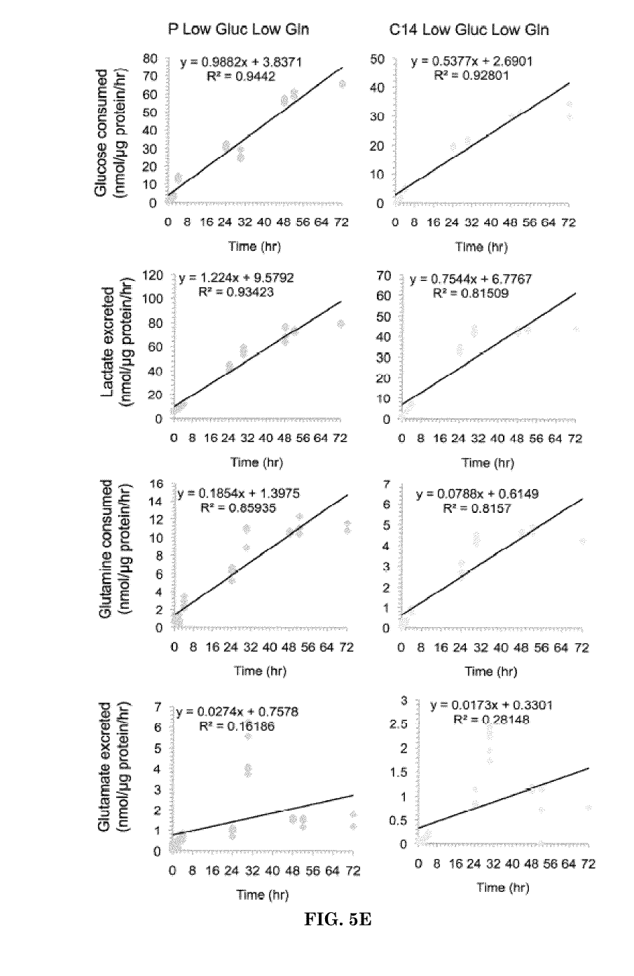

Rapid glycolytic flux in proliferating and quiescent fibroblasts. Previous studies have reported that lymphocytes induced to exit the cell cycle in response to mitogen withdrawal exhibit decreased glycolytic activity. See Bauer D E, Harris M H, Plas D R, Lum J J, Hammerman P S, et al. (2004) Cytokine stimulation of aerobic glycolysis in hematopoietic cells exceeds proliferative demand. Faseb J 18: 1303-1305, which is incorporated herein by reference as if fully set forth. Several methods were used to assess metabolic rates in P, CI7, CI14, and CI14SS7 cells. The rates at which glucose and glutamine were consumed from the medium, and lactate and glutamate were secreted into the medium were monitored. As shown in FIGS. 4A-C, the rate of glucose consumption was approximately two-fold lower in the contact inhibited, relative to proliferating, fibroblasts. Lactate secretion decreased less than two-fold due to contact inhibition alone, and roughly two-fold with additional serum deprivation. Glucose consumption actually slightly increased in fibroblasts induced into quiescence by serum-starvation (without contact inhibition) for 4 or 7 days (FIGS. 5A-E). Metabolic rates were also monitored for fibroblasts cultured in medium conditions containing physiological levels of glucose and glutamine (1 g/l glucose and 0.7 mM glutamine compared with 4.5 g/l glucose and 4 mM glutamine in DMEM (Dulbecco's Modified Eagle Medium, Hyclone, Thermo Fisher Scientific Inc., Logan, Utah)). Metabolic rates were somewhat lower in proliferating fibroblasts in these low glucose-low-glutamine conditions compared with proliferating fibroblasts in standard medium (FIGS. 5A-E). Quiescent fibroblasts cultured in these conditions exhibited consumption and excretion rates approximately half that of proliferating fibroblasts. This finding that glycolytic rates are similar within a factor of two in proliferating and quiescent fibroblasts is surprising given that changes in glycolytic rate have been shown to mirror proliferative rate in multiple model systems. Indeed, while there is a dramatic decrease in the fraction of cells in the proliferative cell cycle, even the CI14SS7 condition resulted in only a 2-fold change in glucose consumption, much less than reported in other systems. Thus, decreased metabolic activity is not a universal hallmark of quiescence.

To further assess glycolytic rates in proliferating and contact-inhibited fibroblasts, the steady state pool sizes of glycolytic intermediates was monitored using liquid chromatography coupled to tandem mass spectrometry (FIG. 1). See Yuan J, Fowler W U, Kimball E, Lu W, Rabinowitz J D (2006) Kinetic flux profiling of nitrogen assimilation in Escherichia coli. Nat Chem Biol 2: 529-530; Munger J, Bajad S U, Coller H A, Shenk T, Rabinowitz J D (2006) Dynamics of the cellular metabolome during human cytomegalovirus infection. PLoS Pathog 2: e132; Lu W, Kimball E, Rabinowitz J D (2006) A high-performance liquid chromatography-tandem mass spectrometry method for quantitation of nitrogen-containing intracellular metabolites. J Am Soc Mass Spectrom 17: 37-50; and Sherr C J, Roberts J M (1999) CDK inhibitors: positive and negative regulators of G1-phase progression. Genes Dev 13: 1501-1512, which are incorporated herein by reference as if fully set forth. In total, levels of 172 metabolites were monitored, 62 of which gave signals above background in P, CI7, and CI14 fibroblasts. Metabolite levels were normalized per microgram of protein in cells plated at the same density because quiescent fibroblasts are smaller and contain less protein per cell than proliferating fibroblasts. The ratio of metabolite levels in the contact-inhibited (CI7 and CI14) to proliferating fibroblasts was determined for each metabolite. Some metabolites were present at consistently higher levels in proliferating fibroblasts, while others were enriched in contact-inhibited fibroblasts, although the magnitude of these changes in metabolite levels was generally modest (FIG. 6).

Levels of five glycolytic intermediates and pentose-5-phosphate (ribose-5-phosphate, ribulose-5-phosphate, and xylulose-5-phosphate, which could not be reliably differentiated in the LC-MS/MS method) are shown in FIG. 4B. No statistically significant differences were observed in the levels of glycolytic intermediates between contact-inhibited (CI7 or CI14) and proliferating fibroblasts at a false discovery rate of 0.05. Some glycolytic metabolites were present at lower levels in contact-inhibited, serum-deprived (CI14SS7) fibroblasts. Thus, the transition between proliferation and quiescence induced by contact inhibition alone has little effect on the pool sizes of glycolytic metabolites in primary fibroblasts. While pool sizes are not a direct indication of changes in flux, the constant levels of glycolytic metabolites in P, CI7 and CI14 fibroblasts are consistent with the finding that there is little change in the rate of glucose uptake or lactate secretion among fibroblasts in these different states.

To more directly assess the rate of flux through glycolytic pathways, fibroblasts were incubated with [U-¹³C]-glucose and it was determined how quickly the label was incorporated into glycolytic intermediates (FIG. 4C). For hexose-phosphate (a combination of glucose-1-phosphate, glucose-6-phosphate and fructose-6-phosphate), FBP, DHAP and PEP, the unlabeled pools of intermediates were converted into fully ¹³C labeled intermediates at a similar rate in P, CI7 and CI14 fibroblasts.

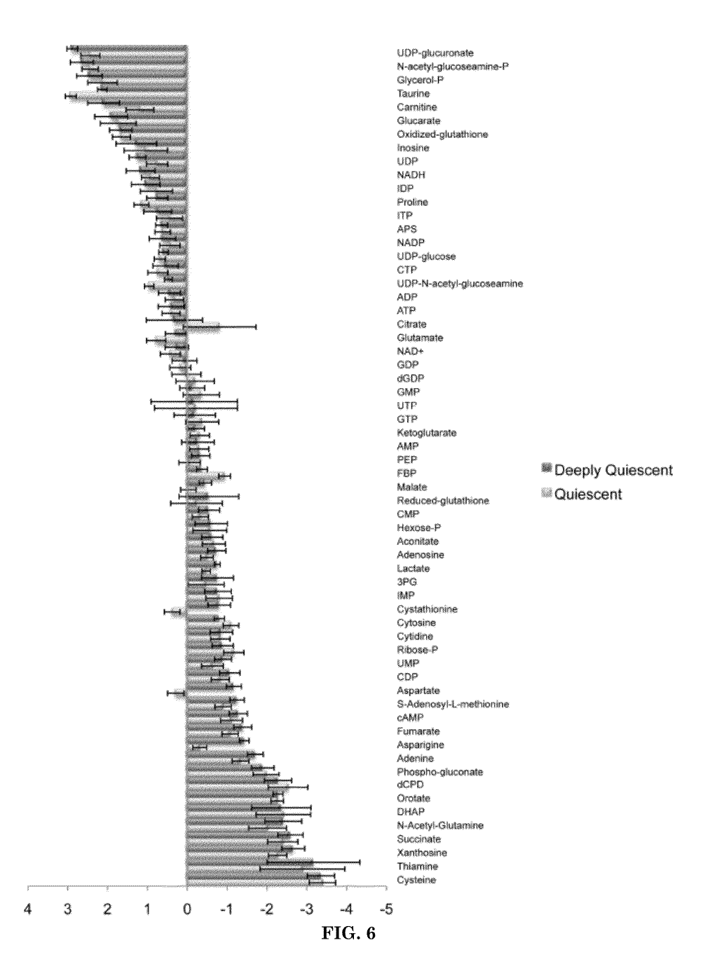

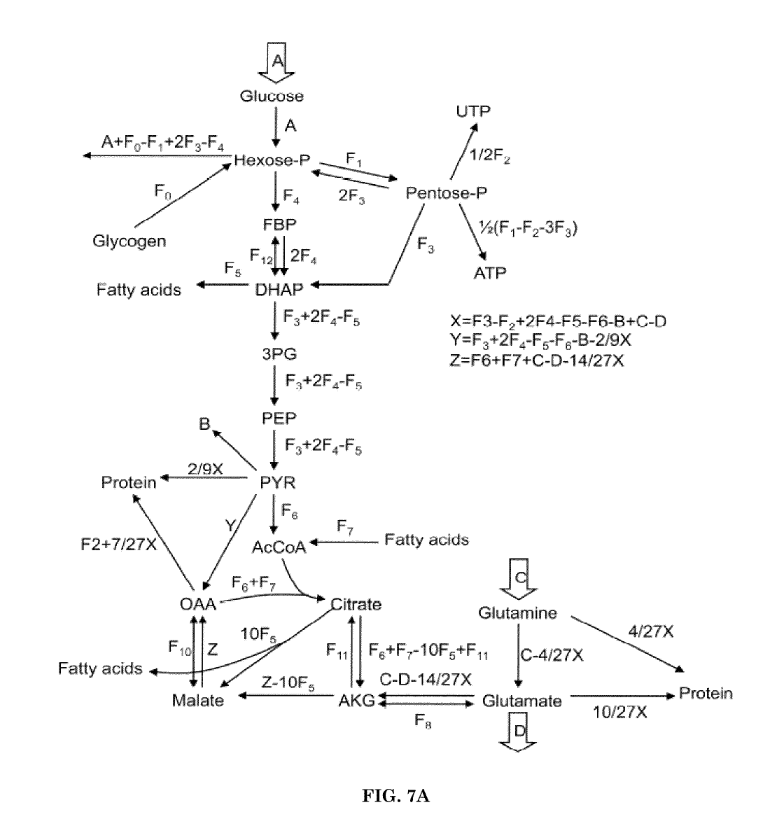

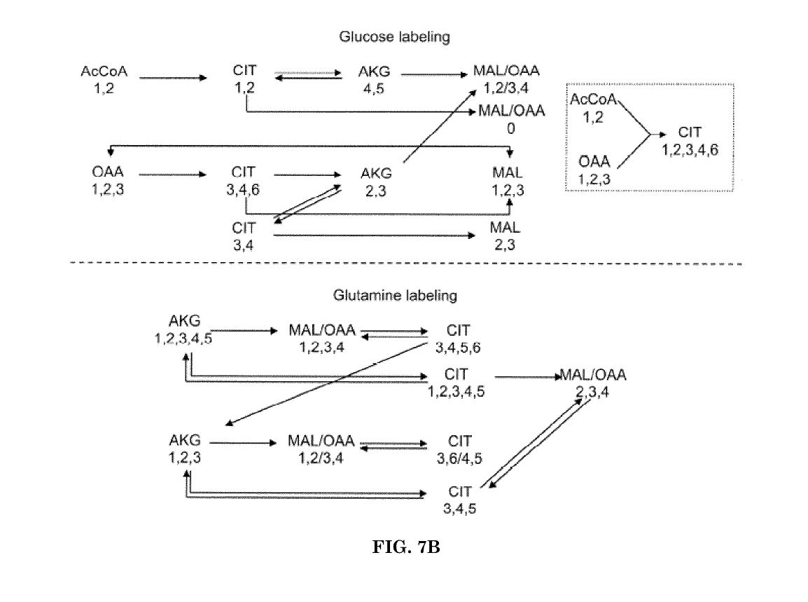

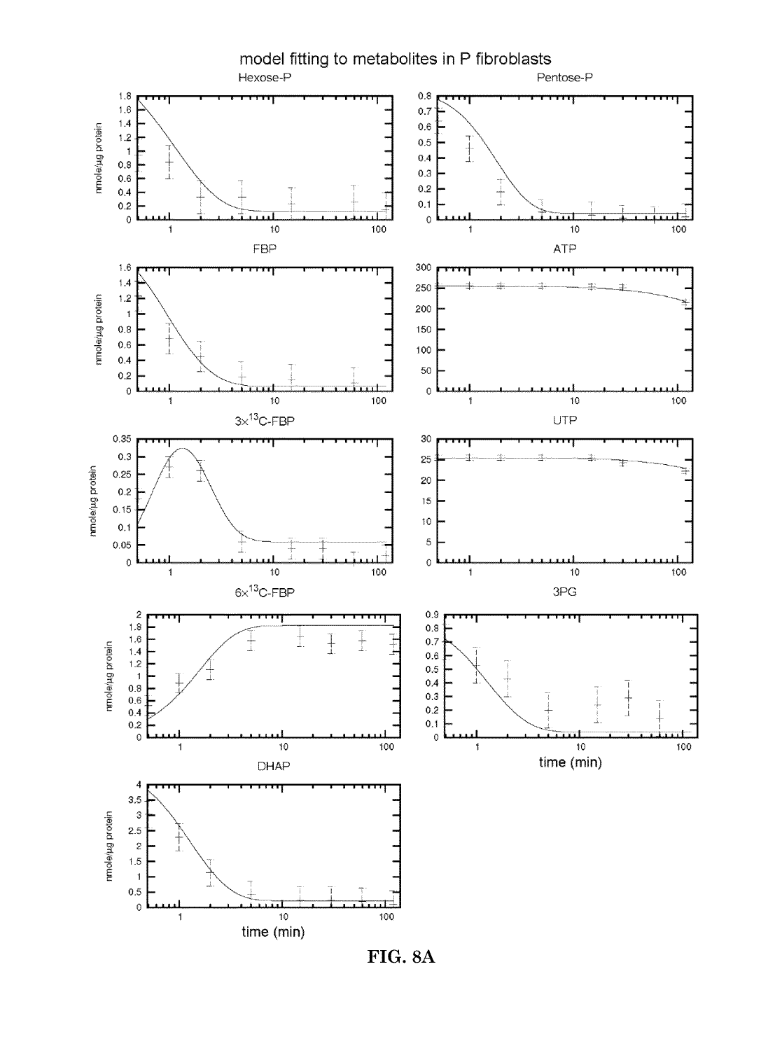

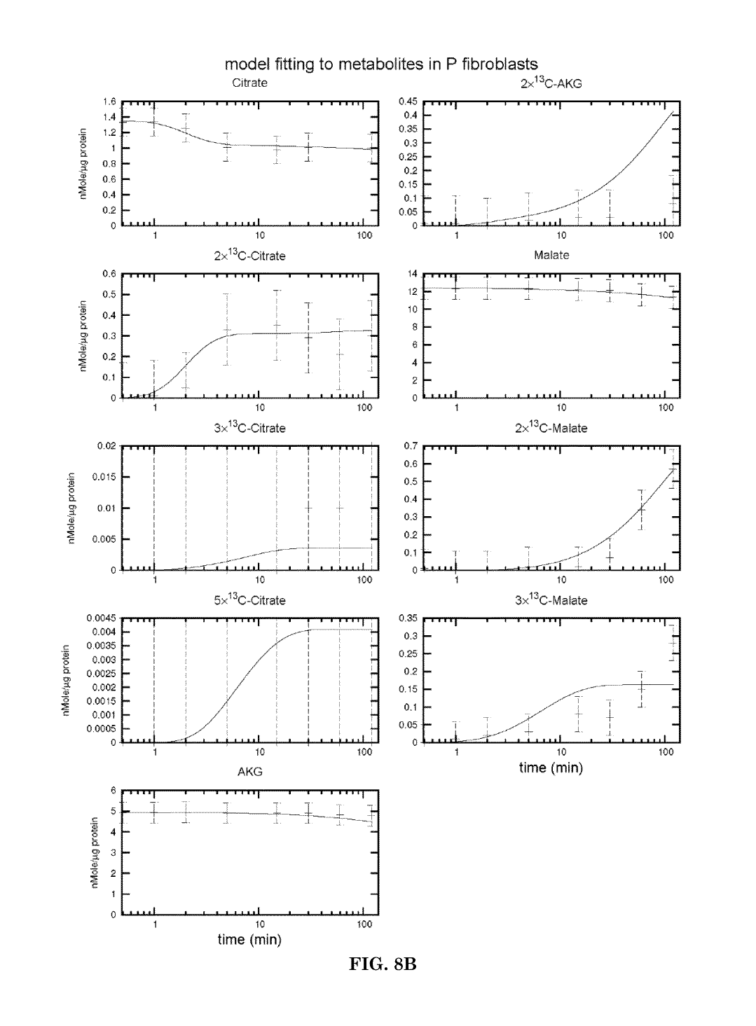

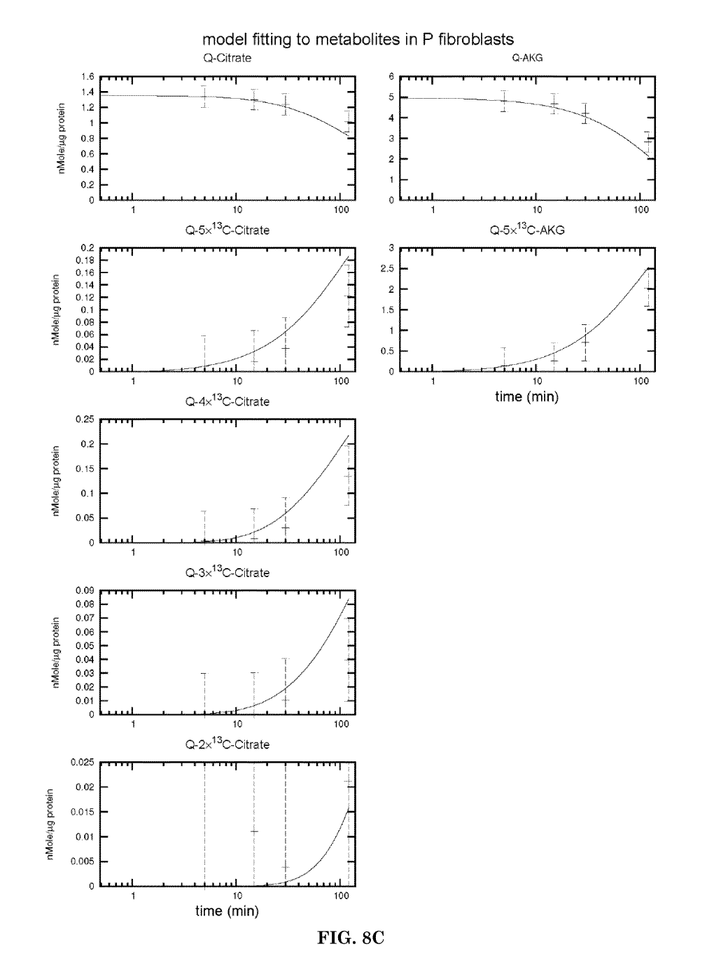

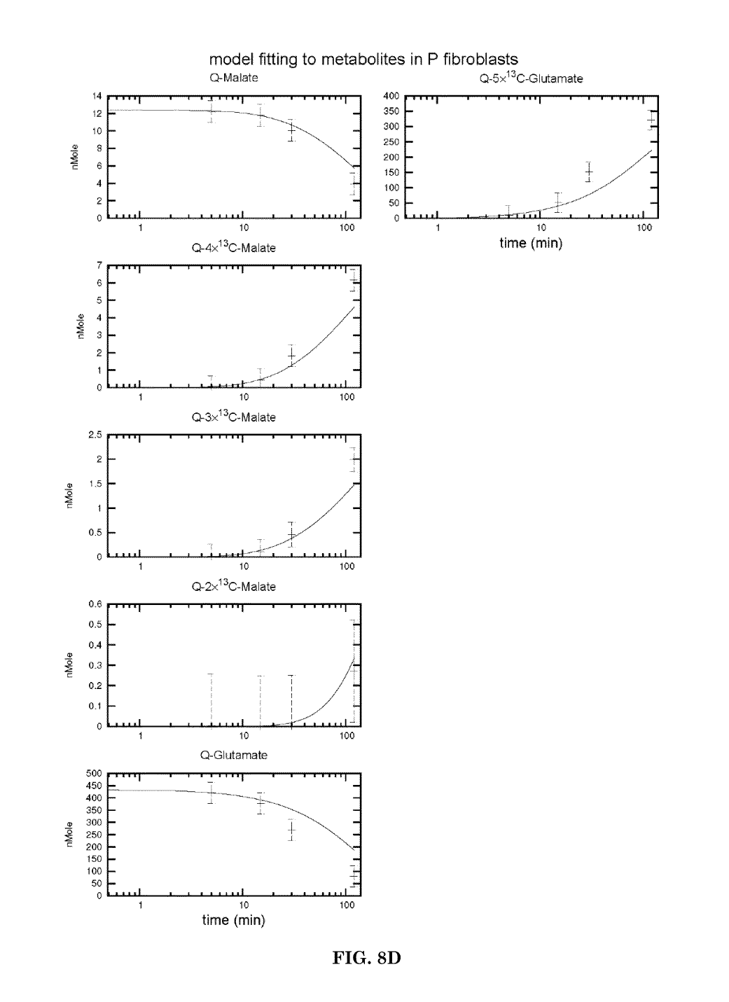

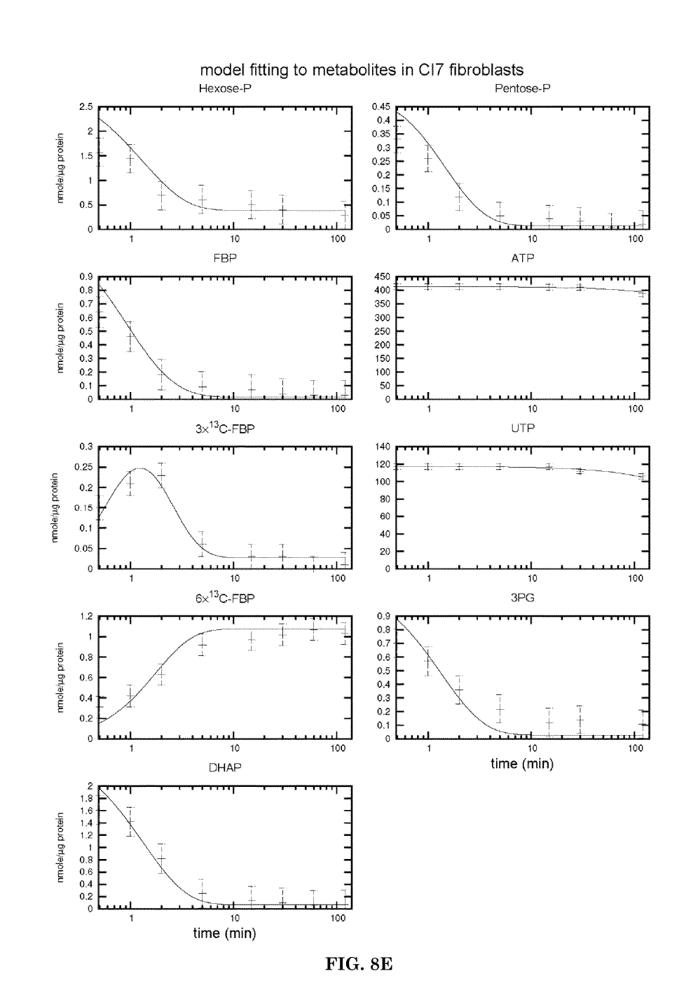

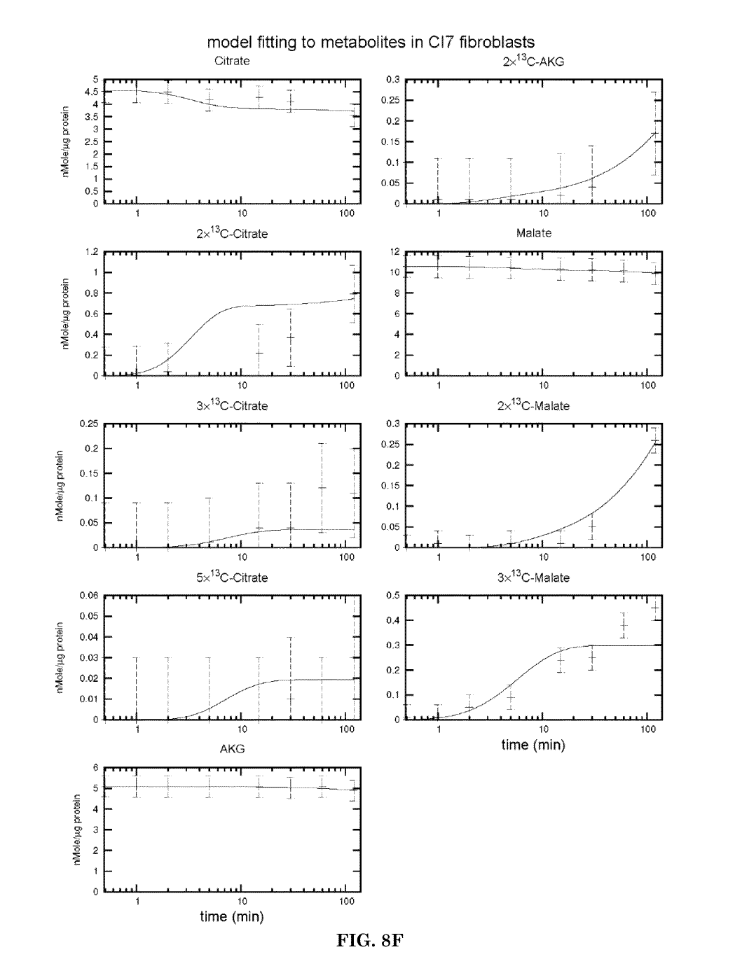

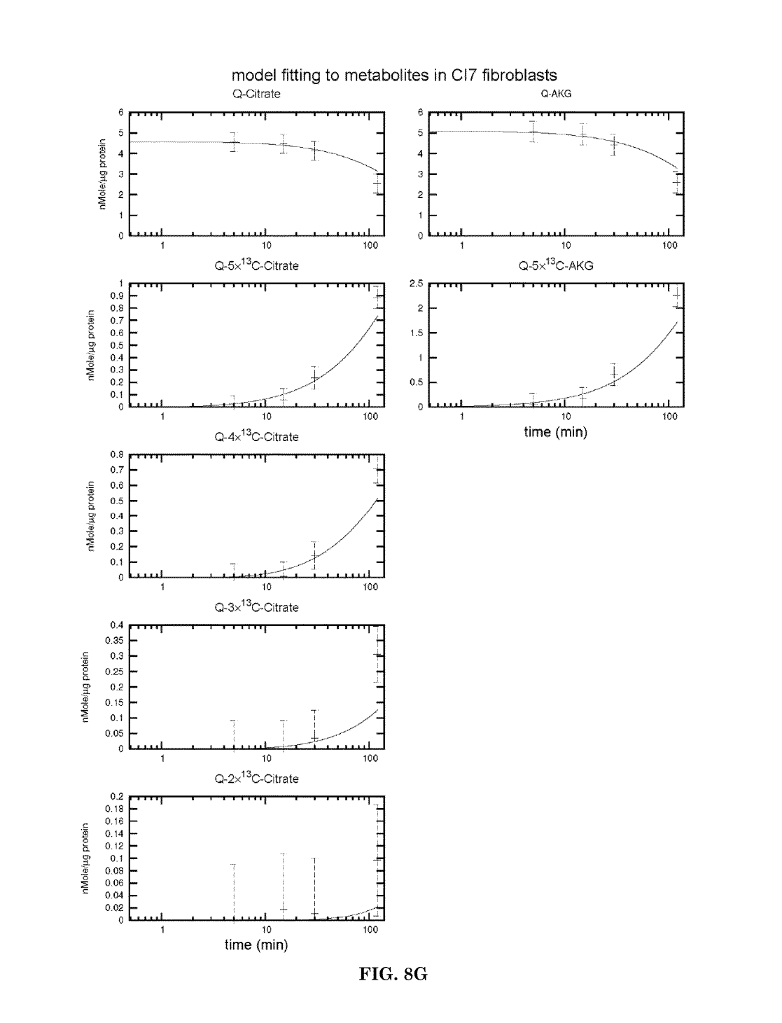

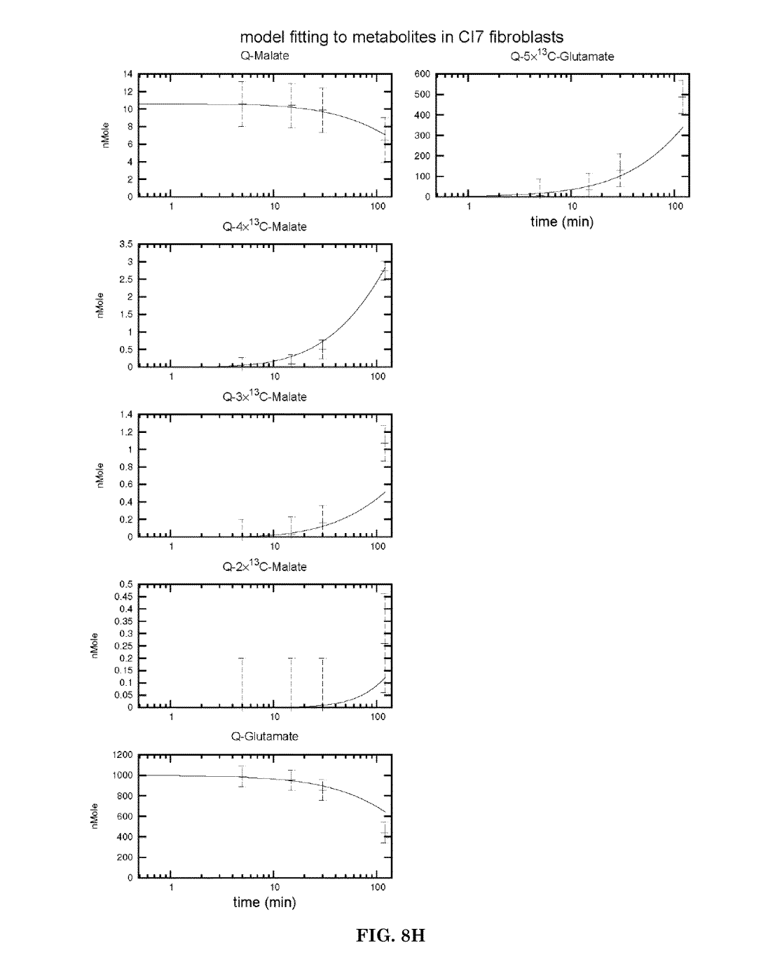

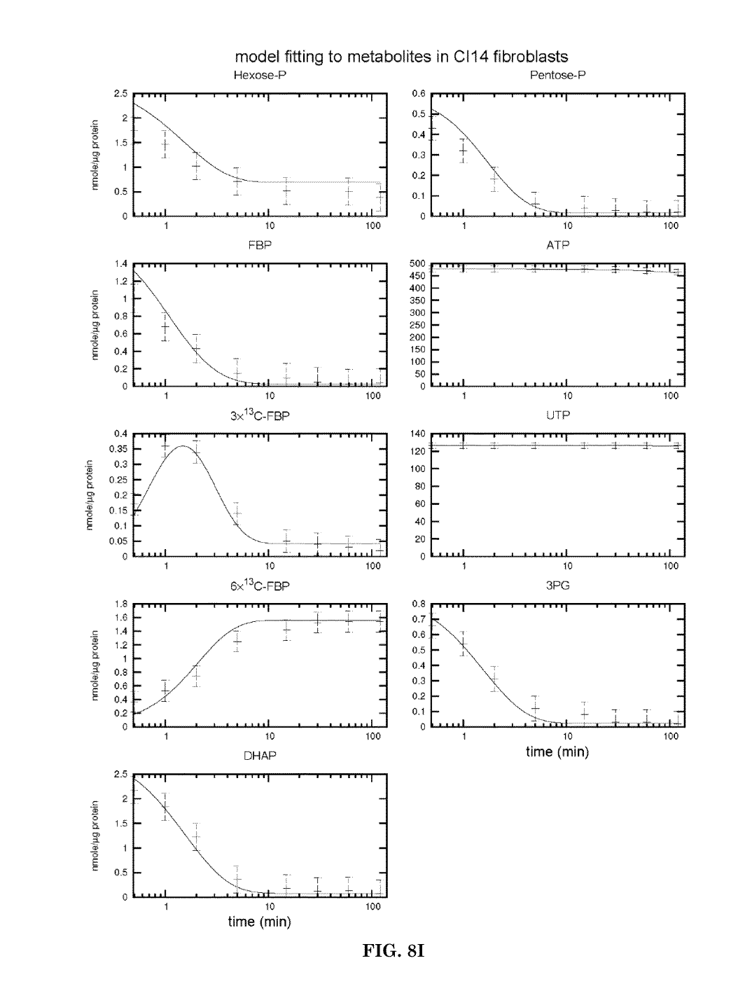

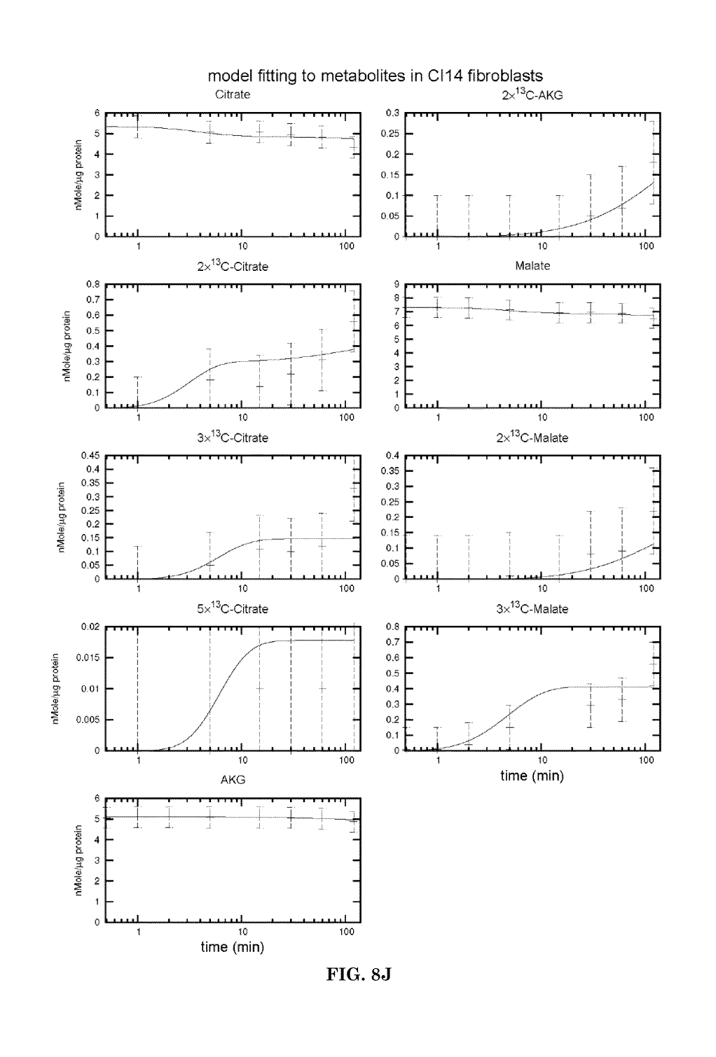

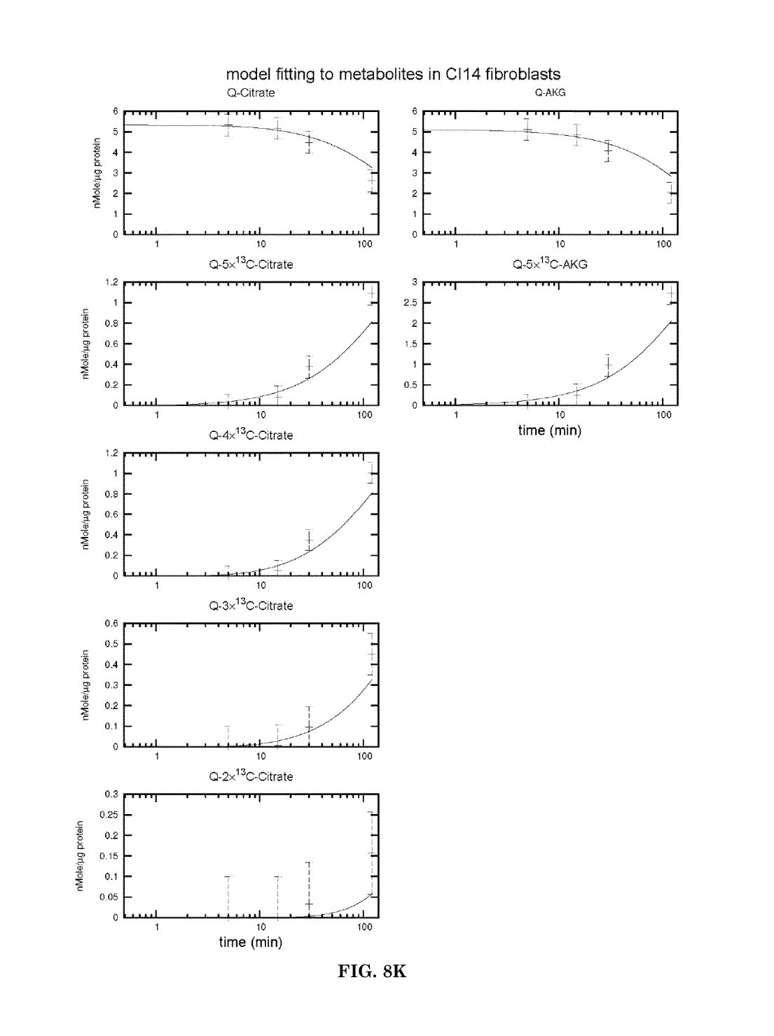

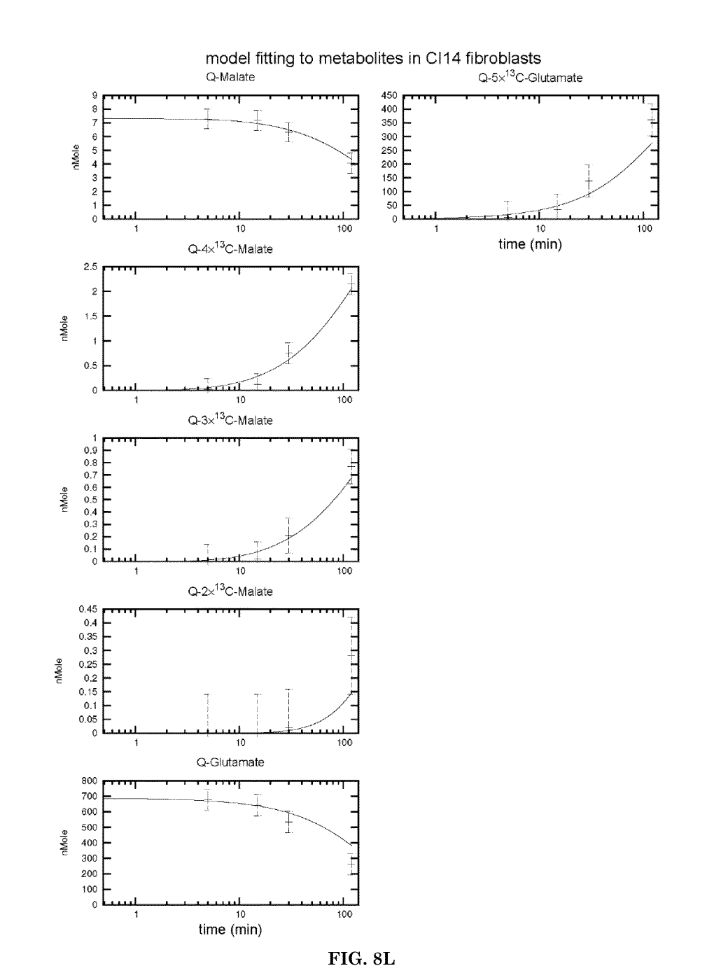

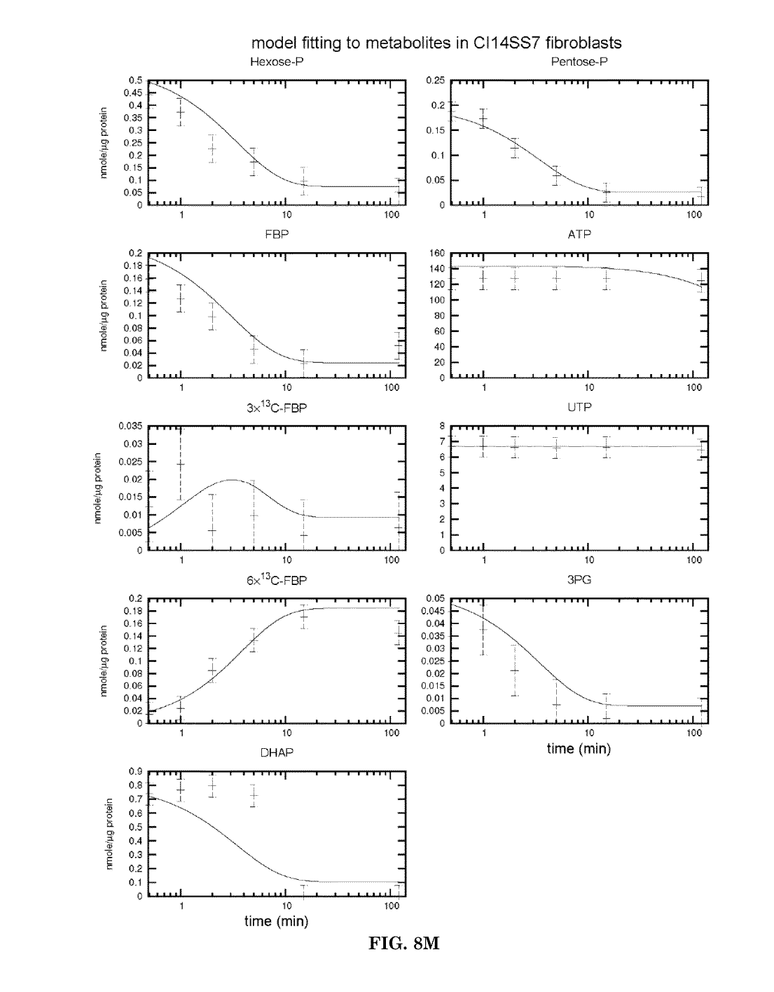

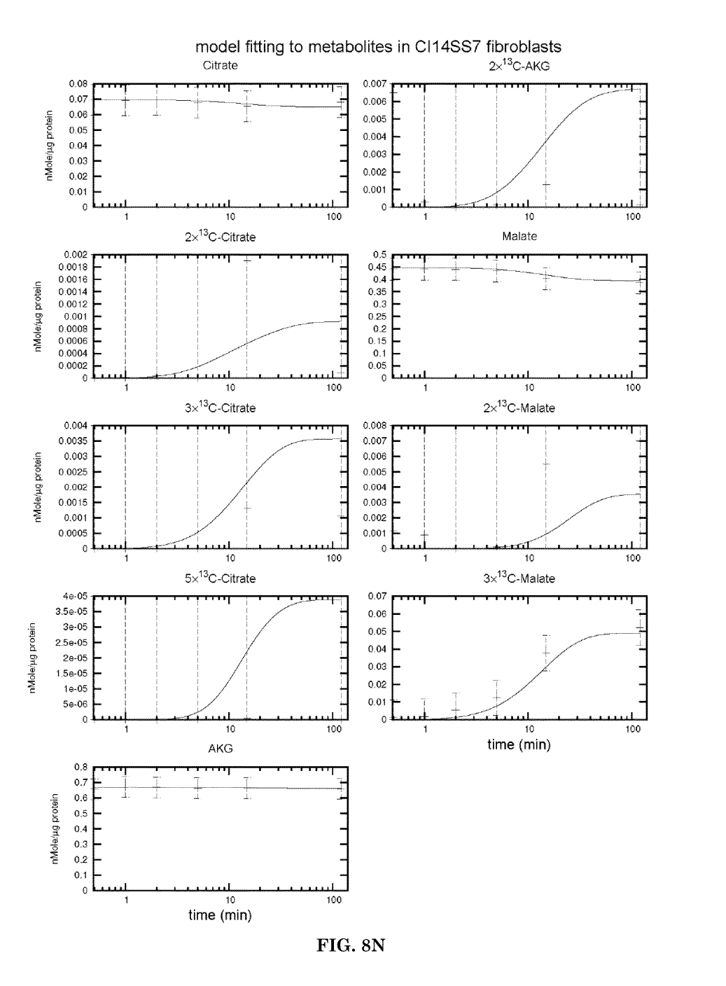

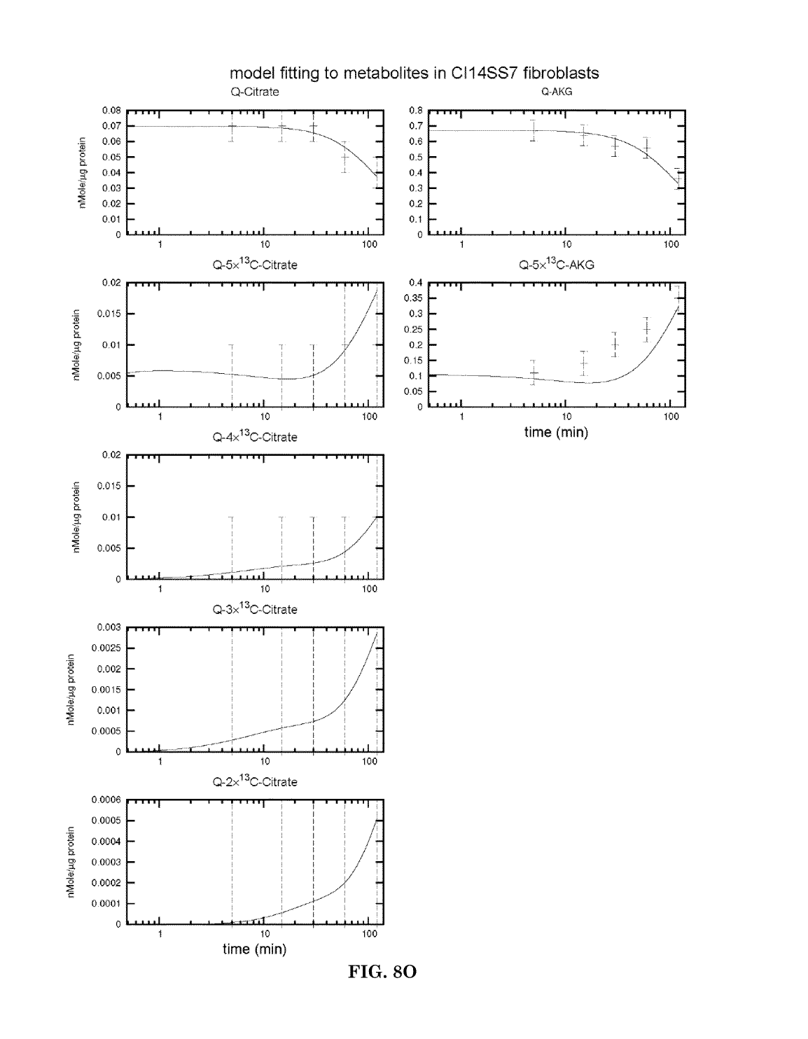

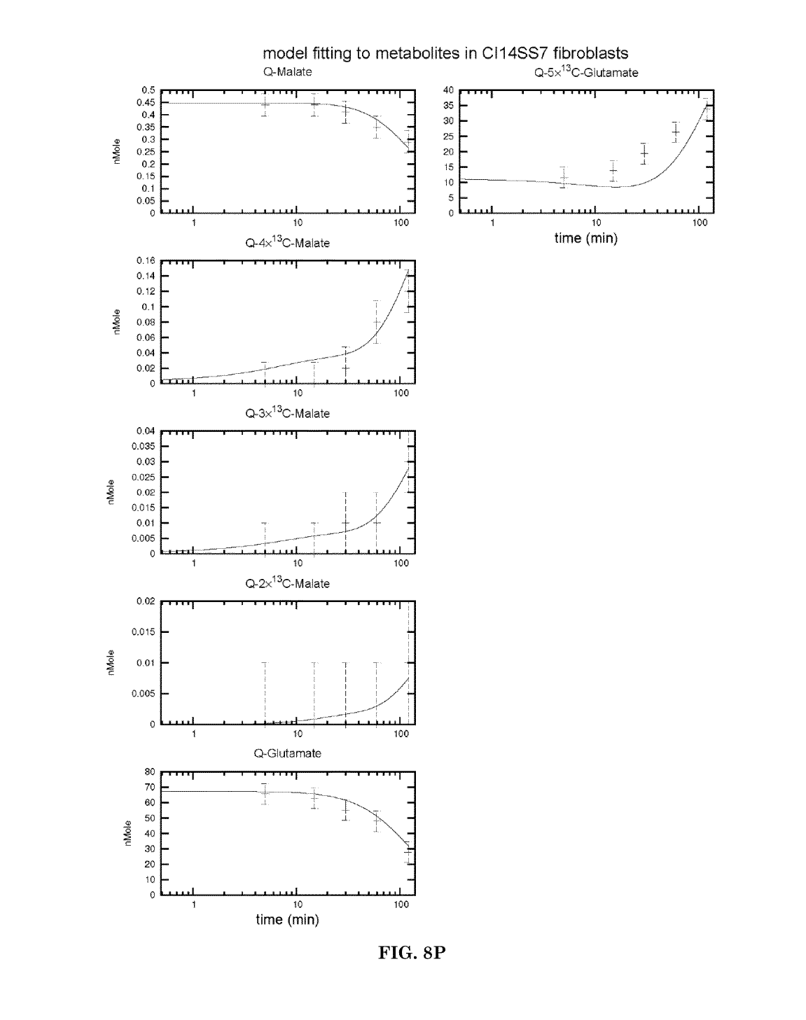

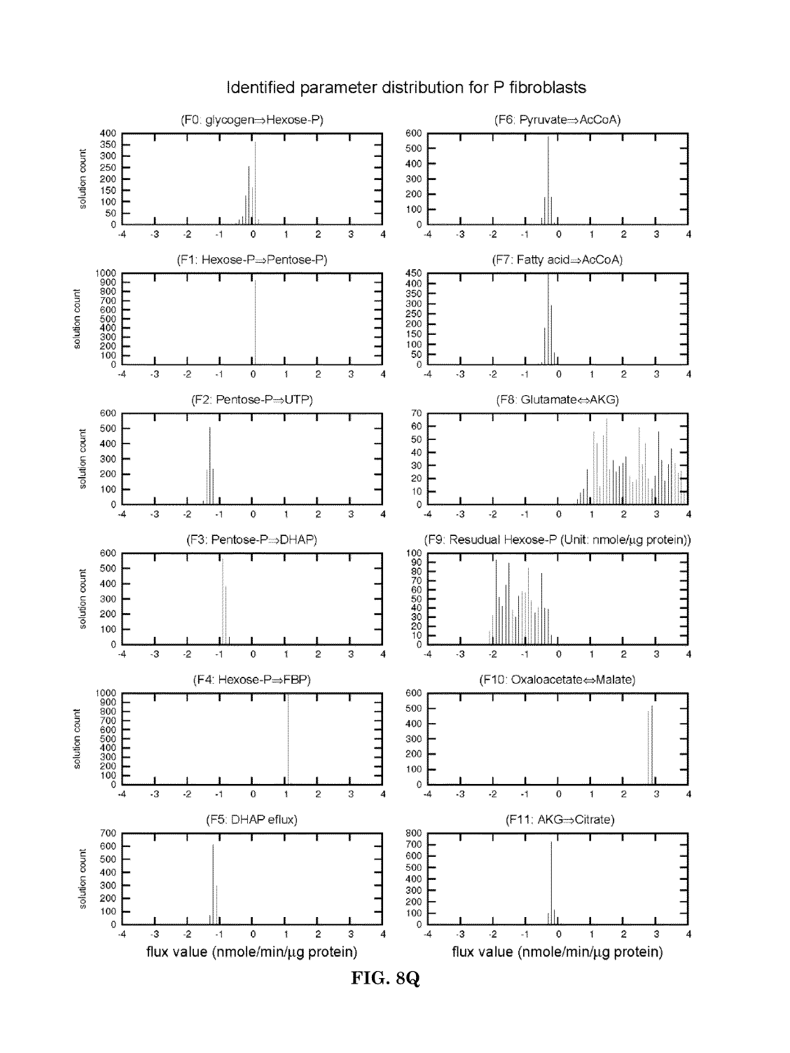

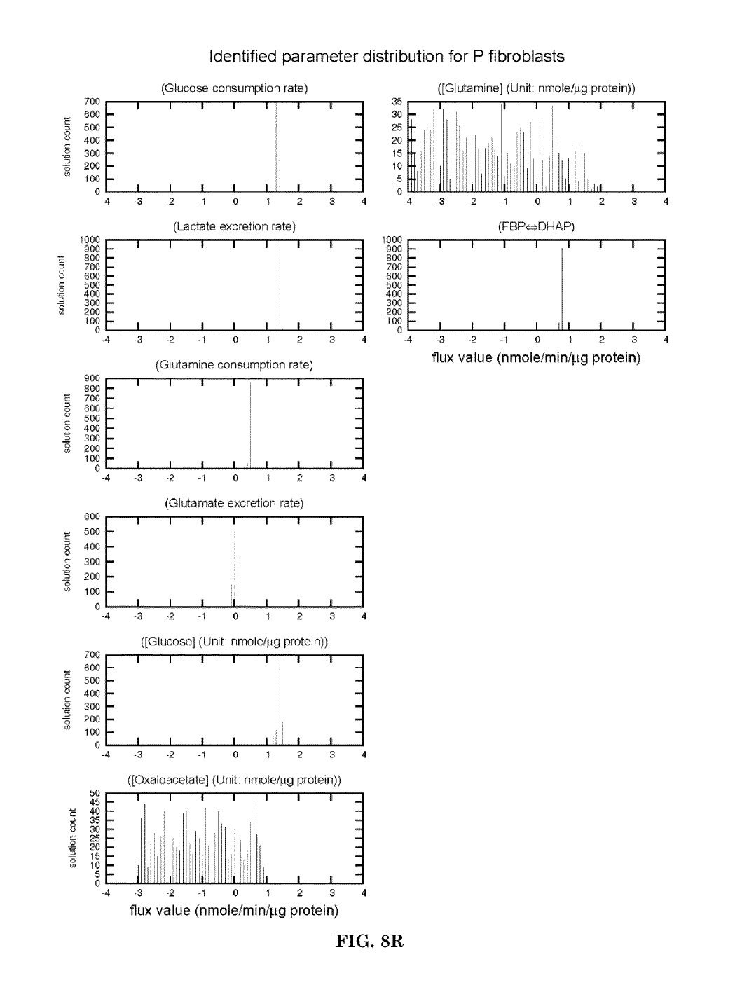

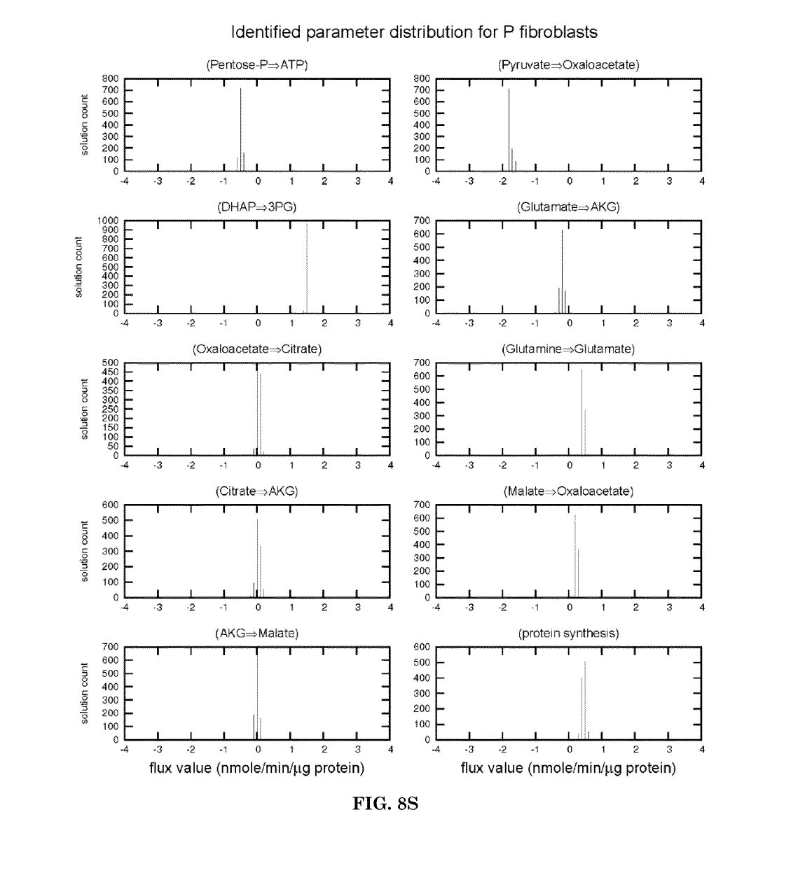

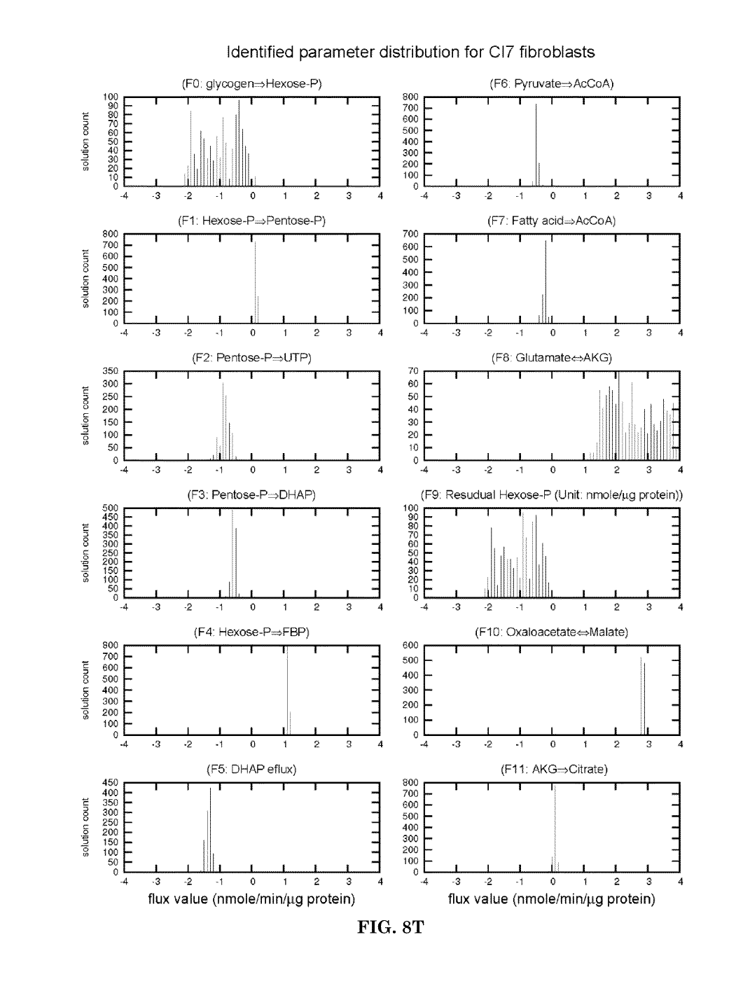

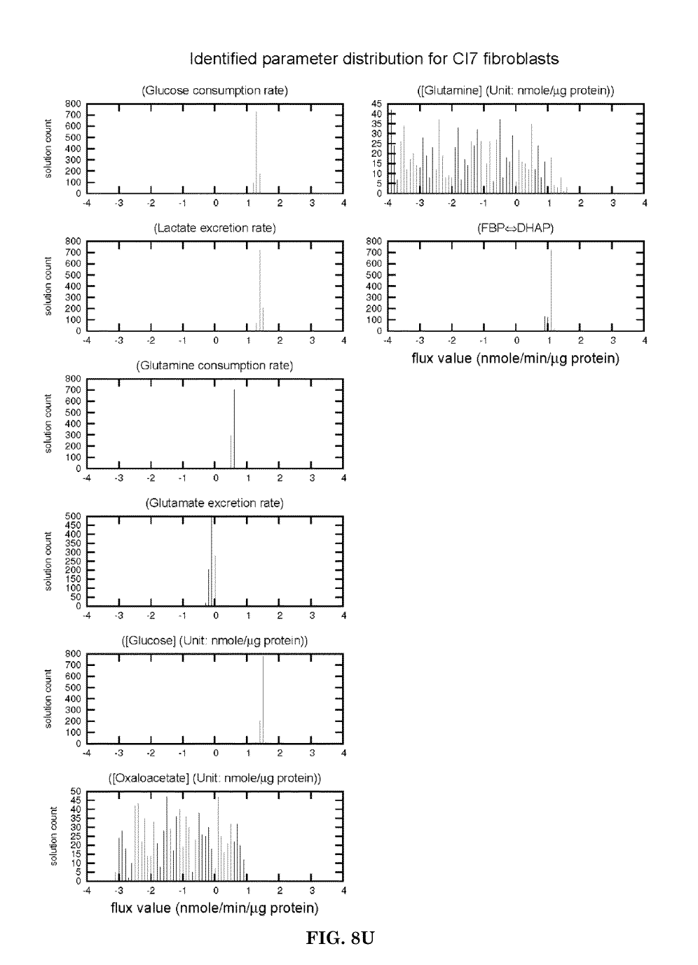

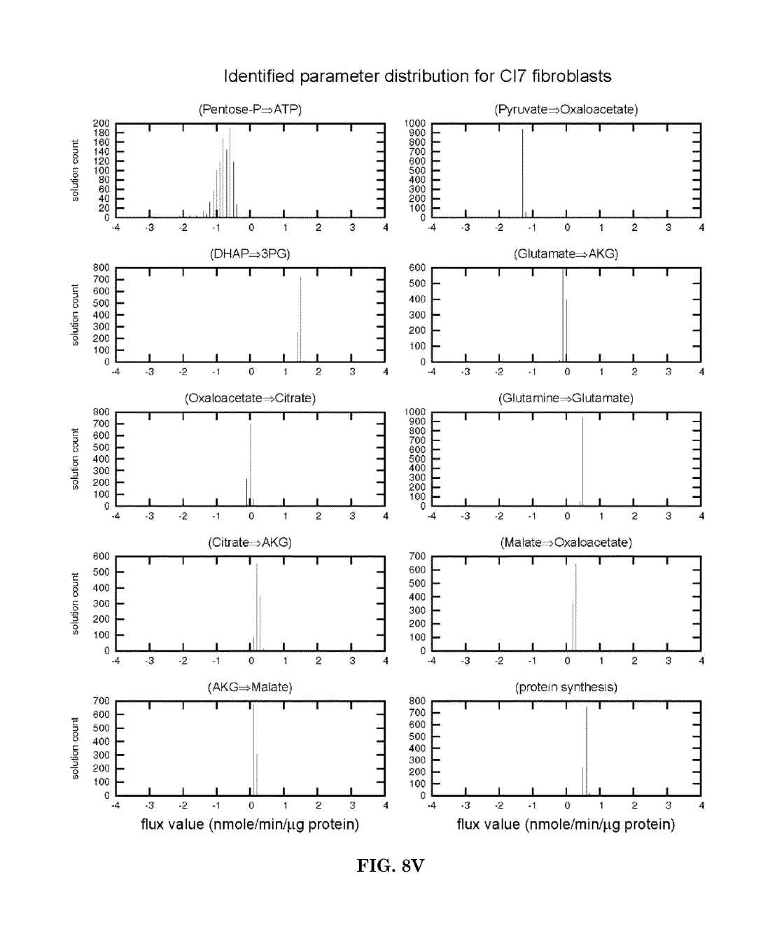

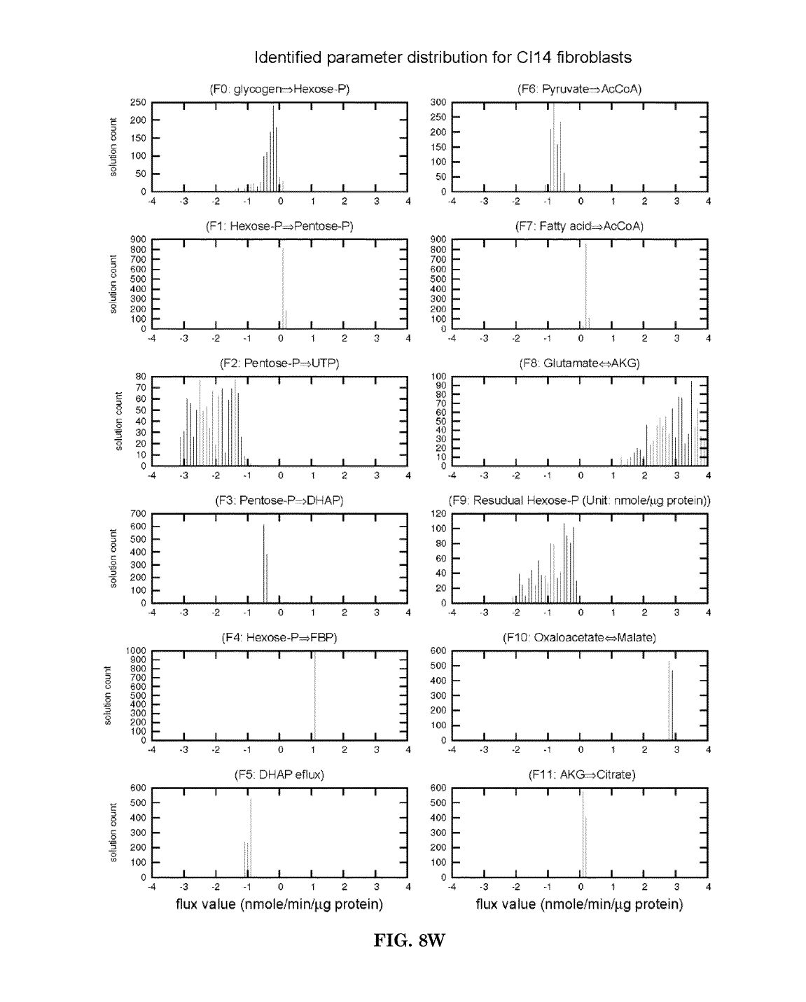

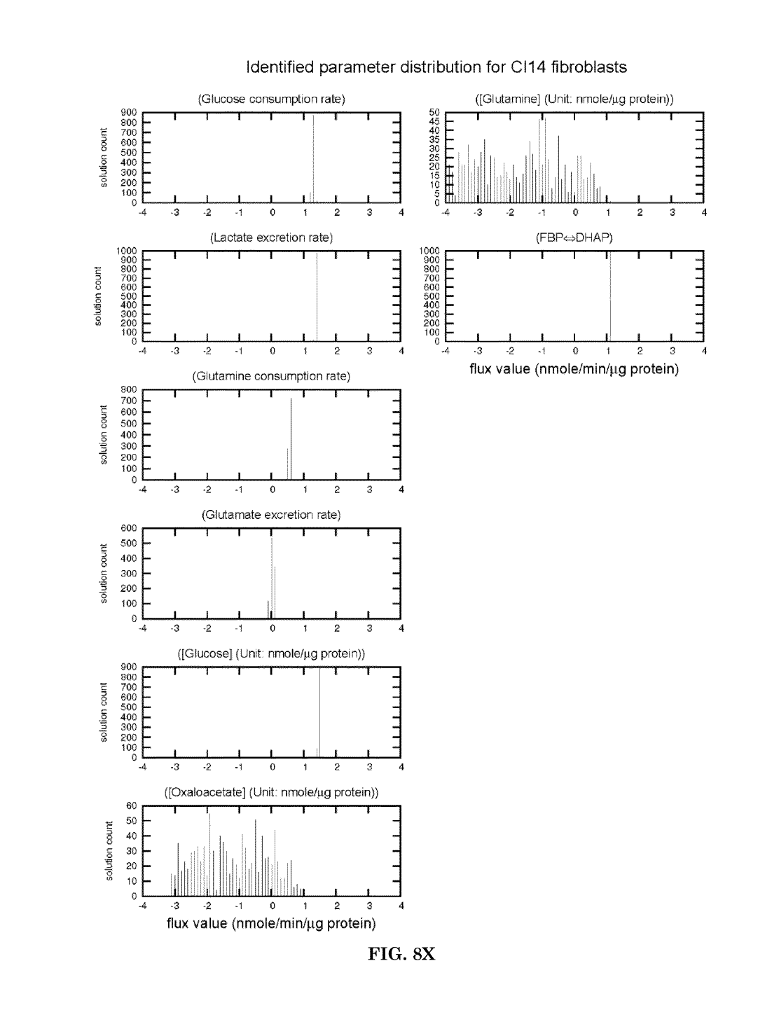

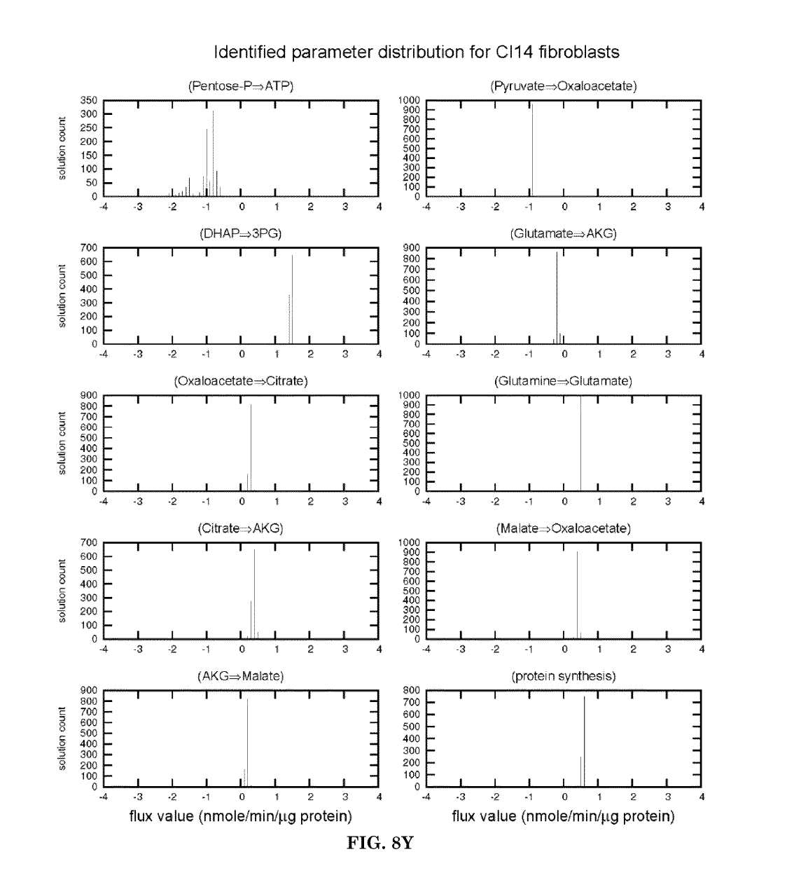

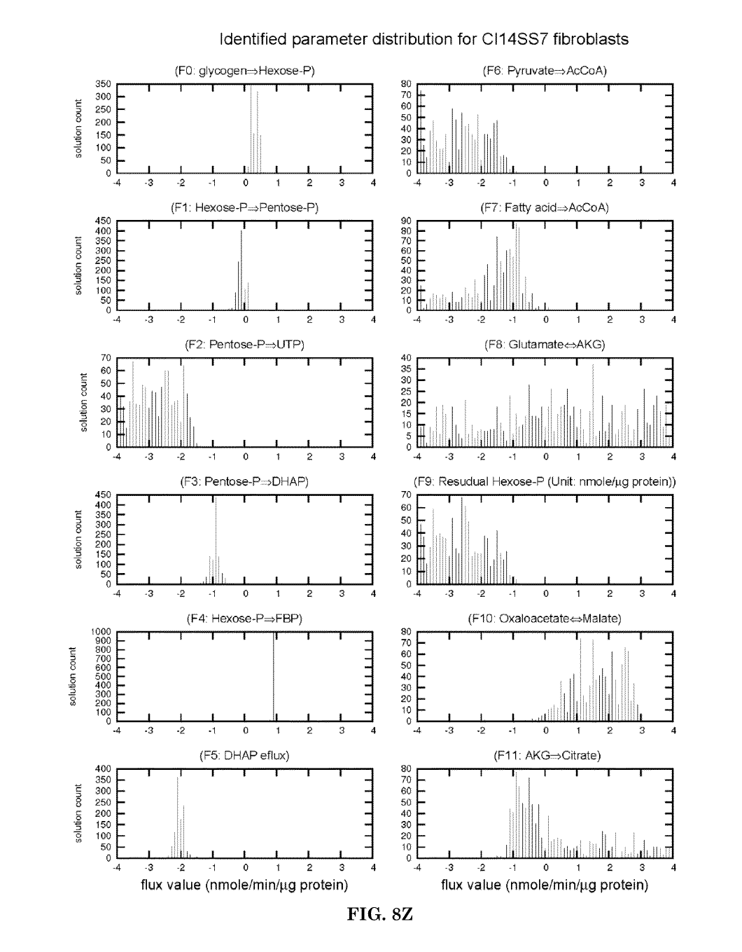

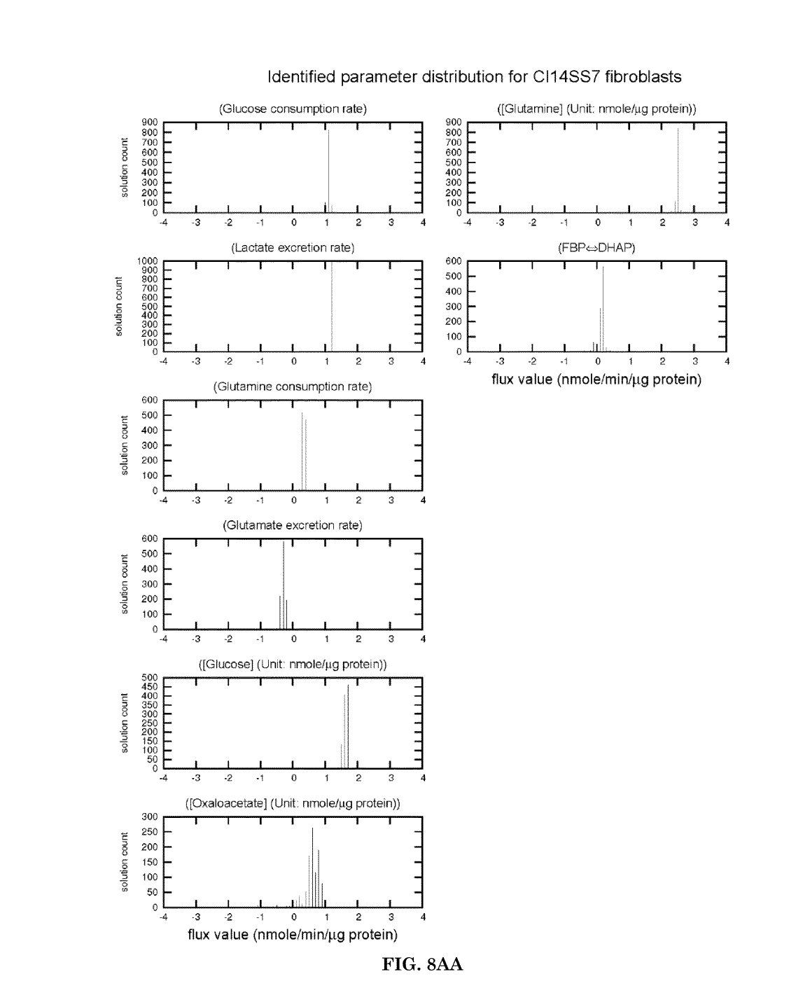

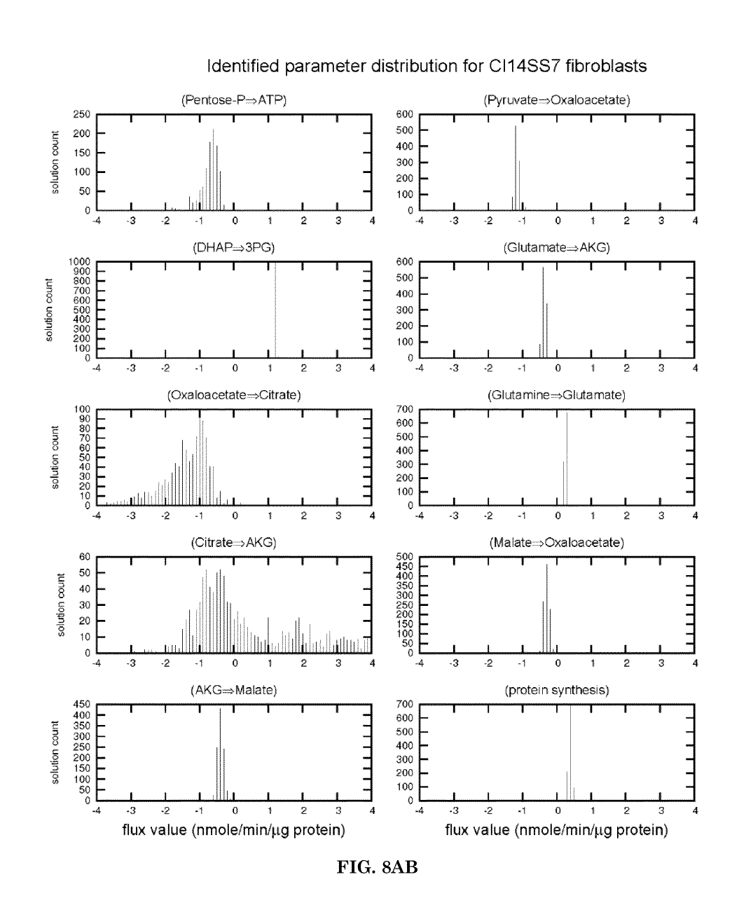

A computational model based on ordinary differential equations (ODEs) of central carbon metabolism for the P, CI7, CI14 and CI14SS7 fibroblasts was developed. The ODEs in the model quantify the isotope labeling dynamics of the relevant metabolites after switching into ¹³C-labeled carbon sources (FIGS. 7A-B). Model parameters (i.e., metabolic fluxes and some unmeasured pool sizes) were identified from fitting all of the available laboratory data (labeling dynamics, pseudo-steady-state labeling patterns, measured pool sizes, uptake and excretion rates). This systems-level approach enabled quantitation of flux through different metabolic pathways in P, CI7, CI14 and CI14SS7 fibroblasts (FIGS. 8A-AB and TABLE 1). For glycolysis, the inferred fluxes from hexose-phosphate to FBP, and from DHAP to 3-phosphoglycerate were similar in P, CI7 and CI14 conditions (FIGS. 8A-AB and 9; see examples below for information regarding statistical significance). In CI14SS7 fibroblasts, hexose-1-phosphate to FBP and DHAP to 3-phosphoglycerate fluxes were approximately half those in the other conditions (FIGS. 8A-AB and TABLE 1), consistent with an approximately two-fold reduction in glucose consumption. It was concluded that glucose consumption and lactate excretion proceed rapidly in fibroblasts induced into quiescence by contact inhibition.

### Table 1

### Absolute Fluxes in Proliferating and Quiescent Fibroblasts

TABLE 1 is split into TABLE 1A and TABLE 1B, below. For each identified flux, the median value of its distribution (TABLE 1A) and the best value (i.e., the one that resulted in the best match between the experimental data and computational simulations) (TABLE 1B) are reported. Flux values that are statistically higher in quiescent than proliferating conditions (i.e., whose distributions do not overlap) are highlighted in bold text, while the fluxes that are lower in quiescent than proliferating conditions are highlighted in italic text.

TABLE 1A
Median values of the absolute fluxes in proliferating
and quiescent fibroblasts.

| Cell type | P | CI7 | CI14 | CI14SS7 |
|---|---|---|---|---|
| Glycogen to HexP | 1.1 | 0.1 | 0.6 | 2.2 |
| Hex-p to RibP | 1.3 | 1.4 | 1.6 | 0.9 |
| RibP to ATP | 0.4 | 0.2 | 0.1 | 0.2 |
| RibP to UTP | 0.06 | 0.2 | 0.01 | 0.002 |
| RibP to DHAP | 0.2 | 0.3 | 0.4 | 0.1 |
| HexP to FBP | 15.8 | 15.8 | 15.8 | 8.9 |
| FBP-DHAP exchange | 7.9 | 14.1 | 12.6 | 1.7 |
| DHAP to 3PG | 31.8 | 31.9 | 32.0 | 18.0 |
| PYR to AcCoA | 0.6 | 0.4 | 0.2 | 0.003 |
| FA to AcCoA | 0.6 | 0.7 | 2.0 | 0.04 |
| PYR to OAA | 0.02 | 0.06 | 0.1 | 0.08 |
| AcCoA to CIT | 1.2 | 1.1 | 2.2 | 0.06 |
| CIT to AKG | 1.2 | 1.9 | 2.6 | 0.7 |
| AKG to CIT | 0.8 | 1.4 | 1.6 | 0.7 |
| AKG to MAL | 1.1 | 1.5 | 1.7 | 0.4 |
| FA synthesis | 0.8 | 0.6 | 1.3 | 0.1 |
| GLT to AKG | 0.7 | 1.0 | 0.7 | 0.5 |
| GLT-AKG exchange | 200 | 316 | 1122 | 2.2 |
| GLN to GLT | 3.1 | 3.5 | 3.4 | 2.1 |
| MAL to OAA | 1.9 | 2.0 | 2.9 | 0.5 |
| OAA-MAL exchange | 891 | 794 | 794 | 56 |
| GLC uptake | 23.8 | 22.9 | 21.9 | 14.2 |
| LAC excretion | 30.4 | 30.5 | 30.7 | 17.3 |
| GLN uptake | 3.6 | 4.1 | 4.0 | 2.5 |
| GLT excretion | 1.2 | 0.9 | 1.2 | 0.6 |
| Protein synthesis rate | 3.2 | 4.3 | 4.1 | 2.8 |

Supplementary Table S1B: Best values of the absolute
fluxes in proliferating and quiescent fibroblasts.

| Cell type | P | CI7 | CI14 | CI14SS7 |
|---|---|---|---|---|
| Glycogen to HexP | 1.1 | 0.6 | 0.6 | 2.0 |
| Hex-p to RibP | 1.3 | 1.8 | 1.8 | 1.1 |
| RibP to ATP | 0.4 | 0.2 | 0.1 | 0.3 |
| RibP to UTP | 0.03 | 0.1 | 0.001 | 0.0002 |
| RibP to DHAP | 0.3 | 0.8 | 1.0 | 0.4 |
| HexP to FBP | 15.8 | 17.8 | 15.8 | 8.9 |
| FBP-DHAP exchange | 7.9 | 14.1 | 12.6 | 2.0 |
| DHAP to 3PG | 31.8 | 35.9 | 32.1 | 18.0 |
| PYR to AcCoA | 0.9 | 0.4 | 0.3 | 0.002 |
| FA to AcCoA | 0.7 | 0.8 | 2.0 | 0.14 |
| PYR to OAA | 0.03 | 0.06 | 0.1 | 0.09 |
| AcCoA to CIT | 1.6 | 1.2 | 2.2 | 0.2 |
| CIT to AKG | 1.6 | 2.1 | 2.6 | 0.2 |
| AKG to CIT | 1.0 | 1.4 | 1.6 | 0.2 |
| AKG to MAL | 1.3 | 1.7 | 1.7 | 0.5 |
| FA synthesis | 1.0 | 0.6 | 1.3 | 0.2 |
| GLT to AKG | 0.7 | 1.0 | 0.7 | 0.5 |
| GLT-AKG exchange | 112 | 89 | 562 | 0.4 |
| GLN to GLT | 3.1 | 3.7 | 3.4 | 2.3 |
| MAL to OAA | 2.3 | 2.3 | 2.9 | 0.6 |
| OAA-MAL exchange | 891 | 794 | 891 | 8.9 |
| GLC uptake | 22.2 | 21.9 | 20.9 | 13.0 |
| LAC excretion | 30.1 | 34.4 | 30.8 | 17.0 |
| GLN uptake | 3.6 | 4.3 | 4.0 | 2.7 |
| GLT excretion | 1.2 | 1.1 | 1.2 | 0.7 |
| Protein synthesis rate | 3.3 | 4.3 | 4.1 | 2.9 |

### Example 12

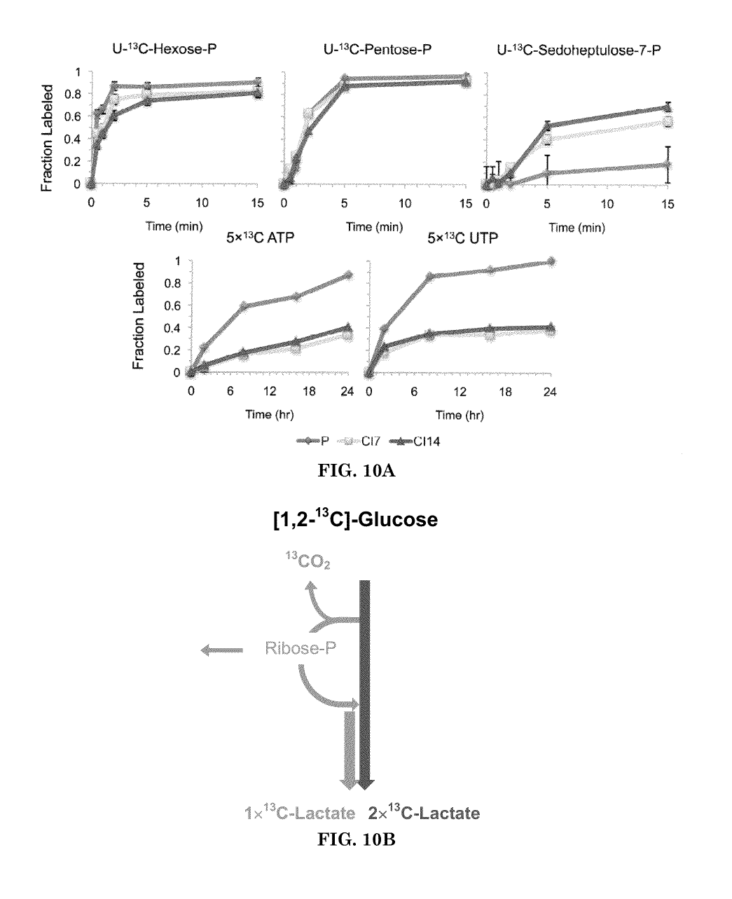

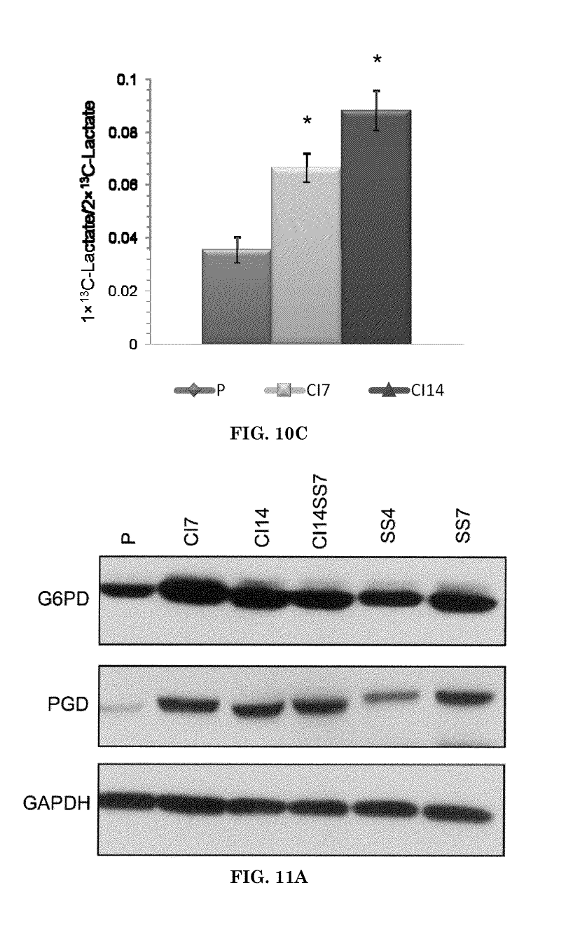

Quiescent fibroblasts exhibit high PPP activity. The PPP produces ribose-5-phosphate needed for the biosynthesis of nucleotides, and NADPH, which can be used as a cofactor for the biosynthesis of macromolecules including fatty acids. It was anticipated that proliferating cells would have higher demands for both ribose-5-phosphate and NADPH than quiescent cells, and thus higher PPP flux. Surprisingly, the pentose phosphate pool incorporated ¹³C label very rapidly in P, CI7 and CI14 fibroblasts when the cells were incubated with labeled [U-¹³C]-glucose (FIG. 10A). Indeed, according to the computational model, hexose-phosphate to pentose-phosphate flux was actually slightly higher in contact-inhibited (both CI7 and CI14) fibroblasts compared with proliferating fibroblasts (though the effect was not statistically significant). Additional serum deprivation only slightly decreased oxidative PPP flux, with the oxidative PPP flux to glycolytic flux ratio highest in CI14SS7 fibroblasts. Thus, the oxidative PPP is actively utilized in both proliferating and quiescent cells.

It was anticipated that ribose generated from the PPP would be incorporated into nucleotide-triphosphates more rapidly in proliferating than quiescent cells due to their increased need for nucleotide triphosphates for RNA and DNA synthesis. Indeed, ATP and UTP with labeled ribose rings accumulate more rapidly in proliferating fibroblasts (FIG. 10A). The results confirm that biosynthesis of nucleotides is more rapid in the proliferating cells.

As discovered and described above, fibroblasts do not commit ribose-phosphate to nucleotide biosynthesis. It was tested whether quiescent cells might recycle ribose-phosphate back to glycolytic intermediates through the non-oxidative branch of the PPP. For this test, the ratio of 1×¹³C-lactate to 2×¹³C-lactate was monitored after incubating the cells with [1,2-¹³C]-glucose. As previously described [27], 1×¹³C-lactate is formed when glucose is metabolized through the oxidative portion of the PPP to ribulose-5-phosphate. In this pathway, glucose molecules lose one ¹³C atom in the form of CO₂, and are then returned to glycolysis through the non-oxidative branch of the PPP (FIG. 10B). 2×¹³C-lactate is formed by the canonical glycolysis pathway from glucose to lactate. The ratio of 1×¹³C-lactate to 2×¹³C-lactate provides an indication of the extent to which the non-oxidative branch of the PPP is utilized. This ratio was significantly higher in CI7 than proliferating fibroblasts, and even higher in CI14 fibroblasts (FIG. 10C).

As another indication of the rate of flux through the non-oxidative branch of the PPP, labeling of sedoheptulose-7-phosphate, a metabolic intermediate in the non-oxidative PPP, was monitored. Sedoheptulose-7-phosphate was labeled rapidly in CI7 and CI14 but not proliferating fibroblasts fed [U-¹³C]-glucose (FIG. 10A) indicating higher flux through the non-oxidative branch of the PPP in quiescent cells. The systems level flux analysis confirmed increased flux from ribose-phosphate back to glycolysis in contact-inhibited compared with proliferating fibroblasts (FIGS. 8A-AB and 9, and TABLE 1). Thus, ribose-phosphate generated from the PPP is utilized for nucleotide biosynthesis in proliferating fibroblasts but is recycled back to glycolytic intermediates in quiescent fibroblasts.

Functional importance of the PPP. To investigate the mechanistic basis for the high PPP flux in quiescence fibroblasts, protein levels of two key enzymes in the PPP monitored. The two key enzymes both generate NADPH, glucose-6-phosphate dehydrogenase (G6PD, Entrez geneID 2539) and 6-phosphogluconate dehydrogenase (PGD, Entrez geneID 5226). Protein levels of both G6PD and PGD were elevated in fibroblasts induced into quiescence by either contact inhibition or serum starvation in comparison to proliferating fibroblasts (FIG. 11A). Serum-starved fibroblasts may activate a program that results in increased levels of PPP enzymes.

Both proliferating and quiescent fibroblasts generate NADPH through the PPP. The NADPH may be used for biosynthesis or to regenerate the reduced forms of glutathione or thioredoxin. The results are consistent with a model in which quiescent fibroblasts up-regulated NADPH production in part to ensure adequate reduced glutathione as protection against free radicals.

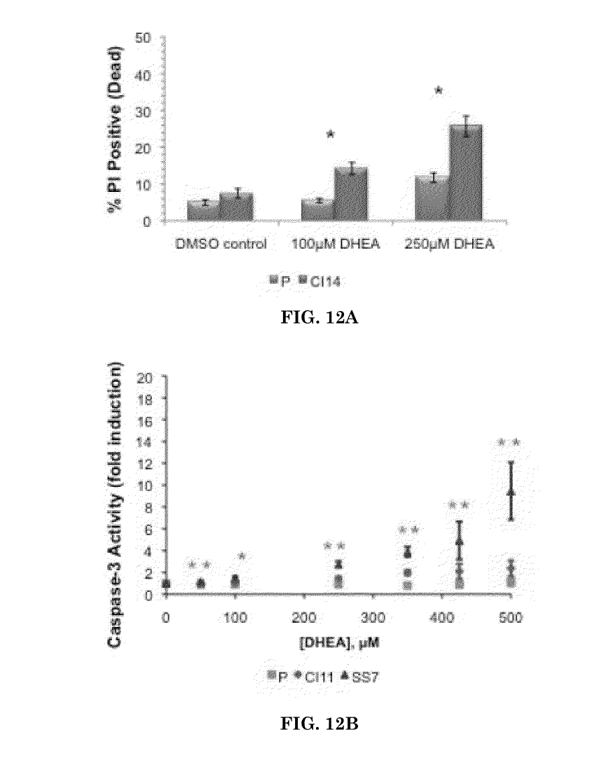

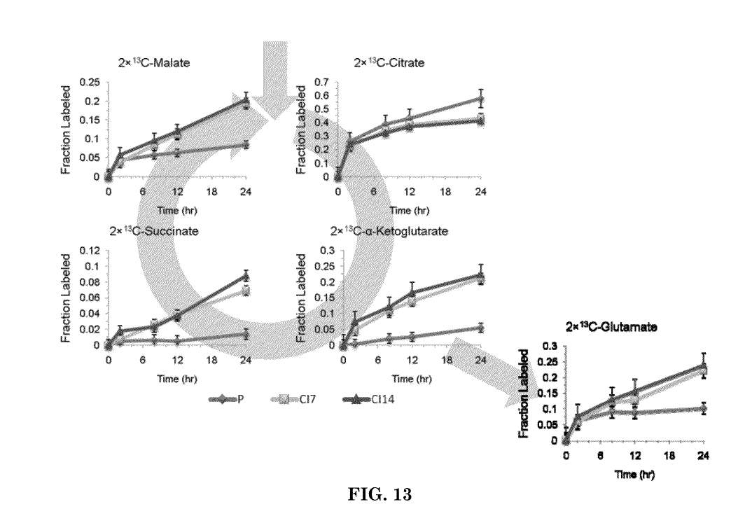

The function of the PPP in quiescent and proliferating fibroblasts was tested. Proliferating or CI14 fibroblasts were incubated with DHEA, a small molecule inhibitor of the PPP for four days. The fraction of cells that were dead was monitored with propidium iodide (PI) labeling followed by flow cytometry. It was discovered that the contact-inhibited fibroblasts exhibited a statistically significant increase in cell death compared with the proliferating fibroblasts from DHEA treatment at 100 μM and 250 μM doses (p<0.01) (FIG. 12A). This result is particularly impressive given that almost all known metabolic inhibitors and cytotoxins preferentially kill proliferating cells. Assaying for caspase 3/7 activity revealed that the mechanism of DHEA-induced cell death in the quiescent fibroblasts is via apoptosis (FIG. 12B). The apoptosis-inducing effect of DHEA was significantly stronger in fibroblasts that were confluent for 11 days than proliferating fibroblasts, and yet stronger in fibroblasts serum-starved for seven days in the absence of contact inhibition.

Considering the relative lack of specificity of the G6PD inhibitor, DHEA, the role of the PPP for quiescent fibroblast survival could be more specifically addressed using G6PD knockdown. Retroviral vectors containing shRNAs that target G6PD could be tested.

### Example 13

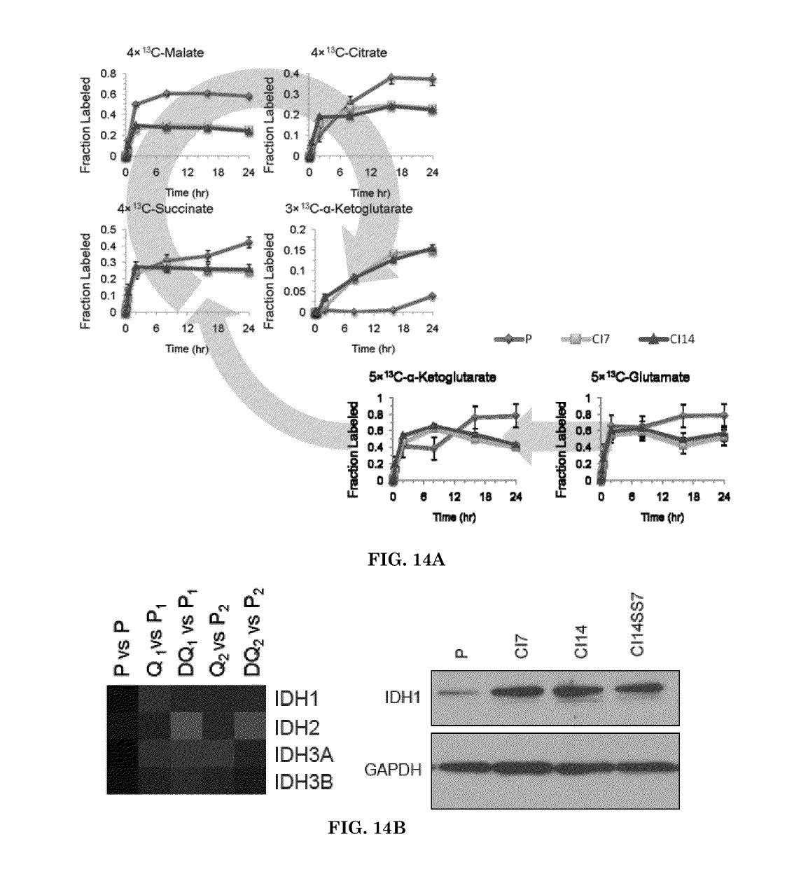

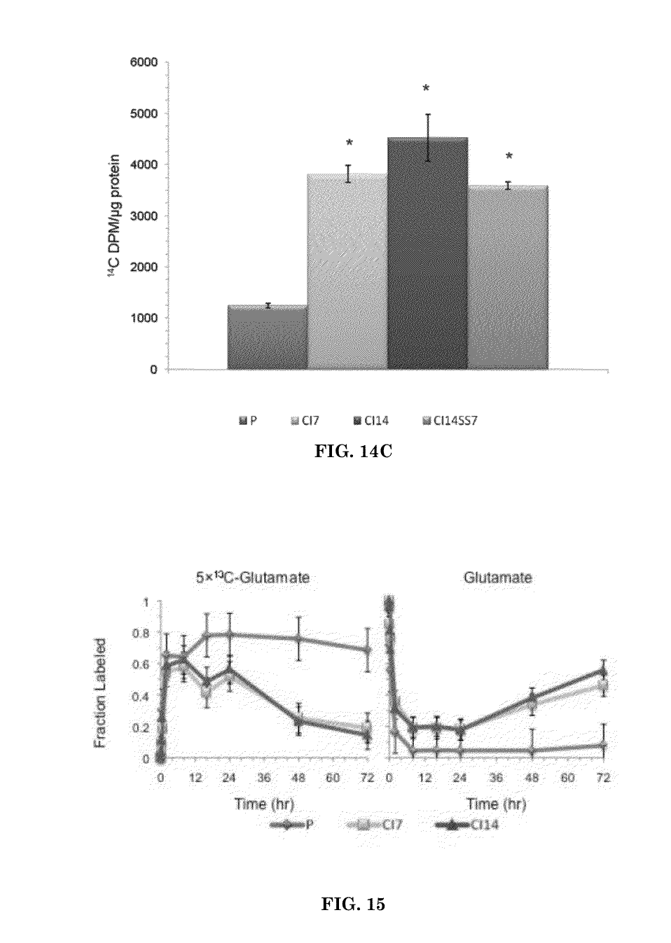

Truncated TCA cycle in proliferating but not quiescent fibroblasts. Previous studies concluded that proliferating lymphocytes actively utilize glycolytic pathways to generate ATP while quiescent lymphocytes generate energy via an influx of fatty acids and proteins that are metabolized through the TCA cycle. To investigate TCA cycle usage, metabolite labeling through the TCA cycle after addition of [U-¹³C]-glucose, [3-¹³C]-glucose and [U-¹³C]-glutamine in P, CI7 and CI14 fibroblasts was monitored. As shown in FIGS. 14A-C, proliferating and contact-inhibited fibroblasts incorporate two carbon units from glucose into citrate via acetyl-CoA at comparable rates. In CI7 and CI14 fibroblasts, the labeled carbons progress through the TCA cycle to form 2×¹³C-α-ketoglutarate as expected. In proliferating fibroblasts, however, there is a substantial decrease in the transmission of labeled carbons from citrate to α-ketoglutarate, succinate, and malate. Experiments using [U-¹³C]-glutamine further support the truncation of the TCA cycle (FIGS. 14A-C). While carbon from glutamine effectively transverses the left side of the TCA cycle in the standard clockwise direction to yield 4×¹³C— citrate in both proliferating and quiescent fibroblasts, subsequent formation of 3×¹³C-α-ketoglutarate by isocitrate dehydrogenase hardly occurs in proliferating fibroblasts. The decreased flux from citrate to α-ketoglutarate in proliferating fibroblasts was confirmed via the systems-level flux identification (FIGS. 8A-AB and 9, and TABLE 1).

When carbon skeletons are removed from the TCA cycle for the synthesis of macromolecular precursors including amino acids, other long carbon skeletons are needed to replace them. This anaplerotic refilling should be especially important for proliferating fibroblasts since their TCA cycle activity is truncated at citrate. The major anaplerotic reaction from glycolysis involves the carboxylation of pyruvate to form oxaloacetate. This reaction can be monitored by feeding cells [3-¹³C]-glucose and monitoring the fraction of citrate or malate with label since the ¹³C is retained only when the anaplerotic reaction via pyruvate carboxylase is utilized. Surprisingly, the ratios of 1×¹³C-citrate to unlabeled citrate and/or 1×¹³C-malate to unlabeled malate were significantly increased in CI7, CI14, and CI14SS7 fibroblasts compared with proliferating fibroblasts (TABLE 2). In addition, quantitative flux analysis revealed that anaplerotic flux from pyruvate to oxaloacetate is elevated in CI7, CI14 and CI14SS7 compared with proliferating fibroblasts (FIGS. 8A-AB and TABLE 1), while the flux from pyruvate to acetyl-CoA is lower in CI14 and CI14SS7 fibroblasts than proliferating fibroblasts. Thus, contact inhibition was associated with both an increase in canonical TCA cycle activity past citrate, and an increase in anaplerotic TCA cycle flux from pyruvate to oxaloacetate. Proliferating fibroblasts, in contrast, seem less likely to have sufficient carbon skeletons from glucose for the production of proteogenic amino acids not present in the cell growth media.

TABLE 2
Malate and citrate labeling after incubation with [3-¹³C]-glucose
in P, CI7, CI14 and CI14SS7 fibroblasts.

| | P | CI7 | CI14 | CI14SS7 |
|---|---|---|---|---|
| 1 × 13C- | 0.0653 ± 0.0120 | 0.0914 ± 0.0155 | 0.0891 ± 0.0163 | 0.119 ± 0.0163 |
| Malate/Malate | | | | |
| p-value compared | — | 0.0198 | 0.00492 | 0.00136 |
| to Proliferating | | | | |
| 1 × 13C- | 0.0956 ± 0.0262 | 0.141 ± 0.0145 | 0.138 ± 0.0209 | 0.126 ± 0.0254 |
| Citrate/Citrate | | | | |
| p-value compared | — | 0.000644 | 0.00207 | 0.0924 |
| to Proliferating | | | | |

### Example 14

Glutamine is the preferred anaplerotic source in proliferating fibroblasts. It was hypothesized that proliferating fibroblasts rely on another source for carbon skeletons. Supplementation with glutamine has been shown to be necessary for cultured cells, especially actively proliferating cells. Accordingly, the rate of glutamine consumption by P, CI7, CI14 and CI14SS7 fibroblasts was monitored (FIGS. 4A and 6A-E). CI7, CI14 and CI14SS7 fibroblasts consume approximately half as much glutamine per microgram of protein as proliferating fibroblasts. CI7 and CI14 fibroblasts secrete glutamate at a lower rate compared with proliferating fibroblasts, and CI14SS7 fibroblasts secrete glutamate at a lower rate than CI7 or CI14 fibroblasts. SS4 and SS7 fibroblasts, on the other hand, consume glutamine and secrete glutamate at a faster rate than proliferating fibroblasts (FIGS. 5A-E). The relative rate of glutamine consumption in P versus CI14 fibroblasts in low glucose/low glutamine conditions is similar to that in standard medium. As shown in FIG. 14A, incubation of P, CI7 and CI14 fibroblasts with [U-¹³C]-glutamine results in rapid labeling of glutamate, α-ketoglutarate, succinate, malate and citrate, indicating that glutamine is used by both proliferating and contact-inhibited fibroblasts for TCA cycle anaplerosis. Since very few glucose carbons are incorporated into the TCA cycle in proliferating fibroblasts, glutamine may serve as the major anaplerotic precursor in proliferating fibroblasts.

### Example 15

Glutamine labeling reveals “reverse” TCA flux. [U-¹³C]-glutamine is converted into 5×¹³C-glutamate and subsequently to 5×¹³C-α-ketoglutarate. 5×¹³C-α-ketoglutarate can proceed through the TCA cycle in the forward direction to generate 4×¹³C-succinate, or alternatively, it can be reductively carboxylated to 5×¹³C-citrate using NADPH as the electron source. Introduction of [U-¹³C]-glutamine led to conversion of −15% of the citrate to the 5×¹³C— form in P, CI7, and CI14 fibroblasts by 8 hours, with more rapid labeling in contact-inhibited fibroblasts. These results support a model in which there is both forward and reverse flux between citrate and α-ketoglutarate, with greater flux in both directions in contact-inhibited than proliferating fibroblasts (FIGS. 8A-AB and 9, and TABLE 1). The forward and reverse flux likely occurs in different compartments, with α-ketoglutarate reductively carboxylated by IDH2 (Entrez geneID 3418) in the mitochondrion, and the resulting citrate reconverted to α-ketoglutarate by IDH1 in the cytosol. As both IDH1 (Entrez geneID 3417) and IDH2 use NADP(H) as their redox cofactor, the net effect is transfer of high energy electrons in the form of NADPH to the cytosol. Consistent with greater flux through this pathway in contact-inhibited fibroblasts, IDH1 protein is increased by contact inhibition at the transcript and protein levels (FIG. 14B). Thus, two major pathways to cytosolic NADPH, the PPP and the IDH2/IDH1 shuttle, are up-regulated at both the protein and flux level in contact-inhibited fibroblasts.

### Example 16

Fatty acid and protein degradation and re-synthesis occur rapidly in proliferating and quiescent fibroblasts. Quiescent cells do not dilute out older macromolecules, organelles or membranes with cell division, and thus may be more dependent than proliferating cells on mechanisms to break-down and re-synthesize membrane components and macromolecules. The data herein are consistent with increased fatty acid degradation in contact-inhibited fibroblasts. Carnitine, a metabolite involved in the transport of fatty acids from the cytoplasm to the mitochondria during fatty acid degradation, is present at higher levels in CI7 and CI14 fibroblasts than in proliferating fibroblasts (FIG. 6). Also, quantitative flux identification revealed, based on long-term labeling patterns of citrate, increased fatty acid breakdown in CI7 and CI14 fibroblasts, but lower rates of fatty acid breakdown in CI14SS7 fibroblasts (FIGS. 8A-AB and 9, and TABLE 1).

The enhanced rate of fatty acid degradation in contact-inhibited fibroblasts may be enabling fatty acid biosynthesis to occur at a similar rate in proliferating and contact-inhibited fibroblasts. During fatty acid synthesis, citrate is transported out of the mitochondria to the cytoplasm where it is broken down by ATP citrate lyase into oxaloacetate and acetyl-CoA used in fatty acid biosynthesis. ATP citrate lyase activity can be monitored based on the conversion of 5×¹³C-citrate to 2×¹³C-acetyl-CoA and 3×¹³C-oxaloacetate (measured as 3×¹³C-malate). 3×¹³C-malate is produced similarly in P, CI7 and CI14 cells, consistent with fibroblasts in all of these states being actively engaged in fatty acid biosynthesis. To more directly assess fatty acid biosynthesis in proliferating and quiescent fibroblasts, lipids were extracted from P, CI7, CI14 and CI14SS7 fibroblasts fed [U-¹⁴C]-glutamine. The contribution of carbons to fatty acids from glutamine was significantly higher in all of the quiescent fibroblasts compared with the proliferating fibroblasts (FIG. 14C), consistent with higher “backwards” flux from α-ketoglutarate to citrate (FIGS. 8A-AB and 9, and TABLE 1). The higher levels of fatty acid synthesis in contact-inhibited fibroblasts may contribute to the maintenance of membrane integrity, and may also provide a major sink for cytosolic NADPH.

The results herein suggest that contact-inhibited fibroblasts may also be actively degrading existing protein, and thus re-synthesizing protein to replace the degraded proteins. As shown in FIG. 15, the fraction of glutamate that is labeled in fibroblasts under all conditions increases rapidly after switching cells into [U-¹³C]-glutamine and then drops off in CI17 and C114 fibroblasts, but not in proliferating fibroblasts. This decline in the fraction of glutamate molecules with five labeled carbons corresponds to an increase in the fraction of unlabeled glutamate. One possible explanation for these data is a breakdown of unlabeled proteins and release of free amino acids into the glutamate pool. These results are in agreement with the quantitative flux analysis: protein synthesis rates are similar across all conditions. (FIGS. 8A-AB and 9, and TABLE 1). Protein synthesis rates in the best fit model are 3.3 nmole/min/μg protein for proliferating fibroblasts, 4.3 nmole/min/μg protein for C17 fibroblasts, 4.1 nmole/min/μg protein for C114 fibroblasts, and 2.9 nmole/min/μg protein for CI14SS7 fibroblasts. Thus, one reason for the active metabolism observed in contact-inhibited fibroblasts may be to rebuild and thus refresh their lipid and protein contents.

### Example 17

Contact-inhibited fibroblasts secrete large amounts of extracellular matrix proteins. The high metabolic activity of quiescent fibroblasts might also be partially explained by their synthesis and secretion of extracellular matrix molecules needed for the structural integrity of tissue. While proliferating fibroblasts would be expected to secrete molecules important for wound healing, quiescent fibroblasts might be expected to secrete extracellular matrix molecules required at the end of a wound healing process or for maintenance of quiescent tissue. The levels of secreted protein in conditioned medium collected from plates containing proliferating or C114 fibroblasts were monitored. Because serum interferes with immunoblotting for specific proteins, these experiments were performed in no serum and 0.1% serum conditions. As shown in FIG. 15, the levels of fibronectin, collagen 21A1 and laminin alpha 2 in conditioned medium from 14-day contact-inhibited (C114) fibroblasts was higher than the levels in conditioned medium from proliferating fibroblasts, thus demonstrating a biosynthetic commitment for contact-inhibited fibroblasts that may contribute to their high metabolic rate.

### Example 18

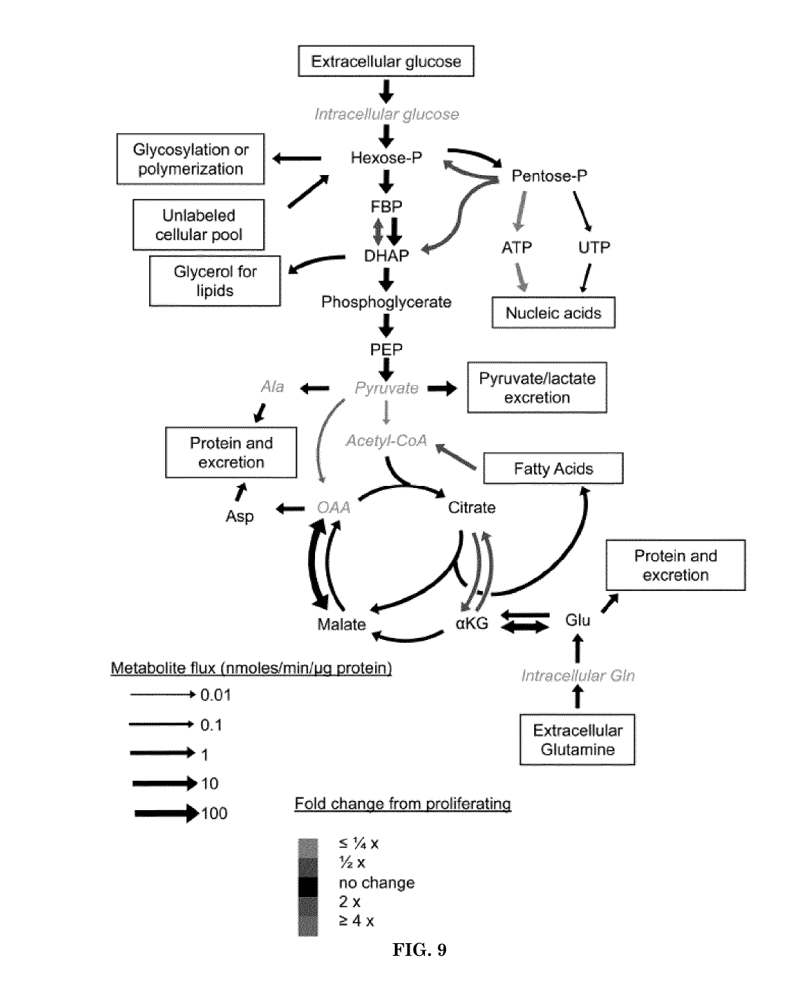

Overview of the metabolic changes between proliferation and quiescence in fibroblasts. The metabolic profiles of proliferating and 14-day contact-inhibited fibroblasts are summarized in FIG. 9. Fibroblasts in both proliferating and contact-inhibited states utilize glycolysis extensively. Proliferating fibroblasts rely on the PPP to generate ribose for nucleotide biosynthesis and NADPH for biosynthetic purposes. Contact-inhibited fibroblasts employ the oxidative PPP to generate NADPH, and the carbon skeletons are largely returned to glycolysis as glyceraldehyde-3-phosphate and fructose-6-phosphate. Fibroblasts in both proliferating and contact-inhibited states contribute some glucose carbons to the TCA cycle. In contact-inhibited fibroblasts, carbons contributed by glucose are transmitted through the TCA cycle; in proliferating fibroblasts, there is little forward flux between citrate and α-ketoglutarate. Contact-inhibited fibroblasts rely more heavily on anaplerotic flux from pyruvate to oxaloacetate via pyruvate carboxylase; proliferating fibroblasts rely more heavily on glutamine, perhaps due to their higher demand for nitrogen. Glutamine drives the forward flux through the TCA cycle and also reverse flux from α-ketoglutarate to citrate, especially in the contact-inhibited fibroblasts. This reverse flux provides a mechanism for shuttling NADPH from mitochondria to the cytosol.

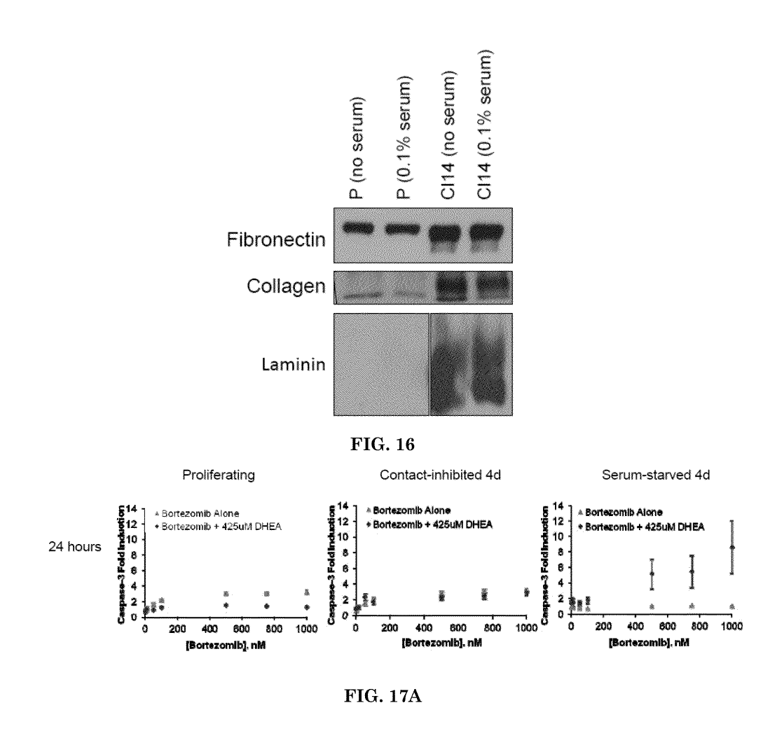

FIG. 16 shows that contact-inhibited fibroblasts secrete high levels of specific extracellular matrix proteins. Four-day conditioned medium was collected from proliferating (P) and 14-day contact-inhibited (CI14) fibroblasts conditioned with either no serum or 0.1% serum, and with 0.03% platelet derived growth factor (PDGF-BB) for proliferating cells. The amount of conditioned medium was normalized to the change in protein content over time. Conditioned medium was precipitated and immunoblotted with an antibody to fibronectin, collagen (col21a1) or laminin (lama2).

### Example 19

### Materials and Methods

Tissue culture: Primary human fibroblasts were isolated from foreskin as previously described. See Legesse-Miller A, Elemento O, Pfau S J, Forman J J, Tavazoie S, et al. (2009) let-7 Overexpression leads to an increased fraction of cells in G2/M, direct down-regulation of Cdc34, and stabilization of Wee1 kinase in primary fibroblasts. J Biol Chem 284: 6605-6609, which is incorporated herein by reference as if fully set forth. Fibroblasts were maintained in DMEM (Dulbecco's Modified Eagle Medium, Hyclone, Thermo Fisher Scientific Inc., Logan, Utah) supplemented with 10% fetal bovine serum (Hyclone) and 100 μg/ml penicillin and streptomycin (Invitrogen Corp., Carlsbad, Calif.). Cells were collected while proliferating, after 1 week of confluent maintenance (CI7), after 2 weeks of confluent maintenance (CI14), after 2 weeks of maintenance with the last 7 days in 0.1% serum (CI14SS7), in 0.1% serum for three days (SS3), in 0.1% serum for four days (SS4) or in 0.1% serum for seven days (SS7). Cells made quiescent by serum starvation alone were plated sufficiently sparsely so that they did not contact surrounding cells. Medium was changed every two days. Proliferating cells were sampled the day after seeding. In order to better simulate conditions in vivo, low glucose/low glutamine conditions were also used in which glucose levels are 1 g/l and glutamine is 0.7 mM compared with a glucose level of 4.5 g/l and a glutamine level of 4 mM in standard DMEM. While cells were confluent, the medium was changed regularly. For analysis, cells were transferred to Dulbecco's Modified Eagle's Medium with 7.5% dialyzed fetal bovine serum (Atlanta Biologicals, Lawrenceville, Ga. or Hyclone) the day before the experiment. Fibroblasts were photographed through a Nikon Eclipse TS100 microscope using a Scion 8-bit color firewire 1394 digital camera. Images were captured with Scion VisiCapture software (Scion Corp., Frederick, Md.).

Flow cytometry for cell cycle: Cells were trypsinized and collected into phosphate-buffered saline (PBS) containing 5% bovine growth serum (Hyclone). Cells were pelleted, resuspended in 67% ethanol in PBS, and stored at 4° C. For flow cytometry, cells were pelleted, washed with PBS, and resuspended in PBS with PI (40 μg/ml) (VWR, West Chester, Pa.) and RNAse A (200 μg/ml) (Thermo Fisher Scientific Inc., Rockford, Ill.). Samples were incubated in the dark for one hour at room temperature, and analyzed using a FACSort flow cytometer (BD Biosciences, San Jose, Calif.). The PI was excited at 488 nm and emitted fluorescence was collected on detector FL2 with a bandpass filter of 585/42 nm. At least 20,000 cells were collected and analyzed with CellQuest software (BD Biosciences). Cell cycle distributions were calculated with ModFit LT software using the Watson Pragmatics algorithm.

Flow cytometry analysis for pyronin Y: To differentiate cells in G₀ versus G₁, fibroblasts representing each quiescence condition were trypsinized and suspended in cold Hank's buffered saline solution (HBSS) at a concentration of 2×10⁶ cells/mL, then added to a fixative of ice cold 70% ethanol. Cells were fixed for at least 2 hours, washed, and re-suspended at 4×10⁶ cells/mL. A solution of 4 μg/mL pyronin Y and 2 μg/mL Hoechst 33342 was added to the cell suspension and incubated on ice for 20 minutes before measuring cell cycle status by flow cytometry. To determine RNA content, pyronin Y was excited at 488 nm and emission was measured at 562-588 nm. DNA content was determined by Hoechst 33342. Excitation was measured at 355 nm and emission was measured at 425-475 nm. Cells in G₀ were identified as the population with 2N DNA content and an RNA content lower than the level in S phase.

Protein content and immunoblot analysis of proliferating and quiescent fibroblasts for p27^{Kip1}, IDH1, G6PD and PGD levels: Cells were made quiescent by contact inhibition, serum starvation or a combination as indicated in the text or figure, and collected at the indicated times. The cells were lysed in RIPA buffer (50 mM Tris-Cl pH 7.4, 150 mM NaCl, 1% Triton X-100, 1% sodium deoxycholate and 0.1% SDS) containing protease and phosphatase inhibitors (10 mM NaPO4 pH 7.2, 0.3 M NaCl, 0.1% SDS, 1% NP40, 1% Na deooxycholate, 2 mM EDTA, protease inhibitor cocktail (Roche, Basel, Switzerland) and Halt Phosphatase inhibitors (Thermo Fisher)). Lysates were sonicated with five pulses for fifteen seconds each at 60 J/W. Lysates were then incubated for thirty minutes on ice with periodic vortexing and cleared by centrifugation for 2-5 min at 4° C. at 10,000 rpm. Total protein amount was assessed by the Lowry method using the BioRad-DC Protein Assay Kit II (BioRad Inc., Hercules, Calif.) as described by the manufacturer. Spectrophotometer readings taken at 650 nm were compared against a standard curve to determine lysate concentration. Total protein content was determined as the product of lysate concentration and lysate volume. Equal amounts of total cellular proteins were resolved on 12% SDS-PAGE and electro-transferred onto a PVDF membrane. Membranes were blocked for 1 hour at room temperature in blocking buffer TBS-T (10 mM Tris pH 7.6, 15 mM NaCl and 0.1% Tween-20) or PBS-T (PBS and 0.1% Tween-20) containing 5% non-fat dried milk. Membranes were incubated with antibodies to p27 (1:500 diluted in TBS-T/5% milk) (Santa Cruz Biotechnology, Santa Cruz, Calif.), IDH1 (1 μg/ml diluted in PBS-T/1% milk) (Lifespan Biosciences, Seattle, Wash.), G6PD (1:1,500 diluted in PBS-T/1% milk) (Novus Biologicals, Littleton, Colo.) or PGD (1:1000 diluted in PBS-T/1% milk) (GeneTex, Irvine, Calif.) overnight. Following incubation, the membranes were washed three times in TBS-T or PBS-T and incubated for 1 hour with horseradish peroxidase-conjugated anti-rabbit secondary antibody (1:3000 diluted into TBS-T/5% milk for p27 or 1:10,000 diluted in PBS-T/1% milk for IDH1 and G6PD) (GE Healthcare, Little Chalfont, Buckinghamshire, UK). The membranes were washed three times with TBS-T or PBS-T and immunoreactive bands were detected with an enhanced chemiluminescence kit (Pierce, Thermo Scientific). The membranes were stripped using Restore Western Blot Stripping Buffer (Thermo Scientific) according to the manufacturer's instruction and immunoblotted with GAPDH (Abcam, Cambridge, Mass.) (1:5000 dilution) in PBS-T/1% milk or TBS-T/5% milk as a loading control.

Intracellular metabolite analysis: Highly parallel measurement of intracellular metabolites was performed as previously described. See Munger J, Bajad S U, Coller H A, Shenk T, Rabinowitz J D (2006) Dynamics of the cellular metabolome during human cytomegalovirus infection. PLoS Pathog 2: e132, which is incorporated herein by reference as if fully set forth. Metabolites were extracted from P, CI7, CI14 or CI14SS7 cells by aspirating the medium from the plate and flash-quenching metabolic activity with 80% methanol maintained at −80° C. Cells were incubated in methanol for 15 minutes, scraped on dry ice and pelleted with centrifugation at 4400 rpm for 5 minutes. Samples were re-extracted twice with 80% methanol on dry ice. The three extractions were pooled and dried under nitrogen gas, dissolved in 300 μl of 50% methanol and spun at 13,000×g for 5 min. Methanol supernatant was then passed through an aminopropyl column. See Bajad S U, Lu W, Kimball E H, Yuan J, Peterson C, et al. (2006) Separation and quantitation of water soluble cellular metabolites by hydrophilic interaction chromatography-tandem mass spectrometry. J Chromatogr A, which is incorporated herein by reference as if fully set forth. Eluate from the column was analyzed with positive ion mass spectrometry via a Finnigan TXQ Quantum Ultra triple-quadrupole mass spectrometer equipped with an electrospray ionization source (Thermo Fisher Scientific Inc.). See Lu W, Kimball E, Rabinowitz J D (2006) A high-performance liquid chromatography-tandem mass spectrometry method for quantitation of nitrogen-containing intracellular metabolites. J Am Soc Mass Spectrom 17: 37-50, which is incorporated herein by reference as if fully set forth. A TSQ Quantum Discovery MAX mass spectrometer, also equipped with an electrospray ionization source, was used to collect data on negative mode ions after separation on a cm C18 column coupled with a tributylamine ion pairing agent to aid in the retention of polar compounds. See Luo B, Groenke K, Takors R, Wandrey C, Oldiges M (2007) Simultaneous determination of multiple intracellular metabolites in glycolysis, pentose phosphate pathway and tricarboxylic acid cycle by liquid chromatography-mass spectrometry. J Chromatogr A 1147: 153-164; and Lu W, Bennett B D, Rabinowitz J D (2008) Analytical strategies for LC-MS-based targeted metabolomics. J Chromatogr B Analyt Technol Biomed Life Sci 871: 236-242, which are incorporated herein by reference as if fully set forth.

To quantify metabolites, peak heights were initially assigned using XCalibur software (Thermo Fisher Scientific Inc.) and then evaluated manually. Metabolites not enriched at least 5-fold in a sample compared with a control plate containing only media were eliminated from analysis. Of the 172 metabolites monitored, 62 met these criteria. Signals that were below the limit of detection were assigned 100. Metabolite levels were normalized by the amount of protein present.

Metabolic flux analysis: To monitor the flux through metabolic pathways, samples were incubated with medium containing isotope-labeled nutrient for different amounts of time. Dulbecco's medium lacking glucose and glutamine was isotope-labeled by adding back glucose or glutamine ([U-¹³C]-glucose, [1,2-¹³C]-glucose, [3-¹³C]-glucose or [U-¹³C]-glutamine, Cambridge Isotope Laboratories, Andover, Mass.) to a final concentration of 4.5 g/L glucose or 0.584 g/L glutamine. Samples were taken at the indicated time points after medium change and processed as described above. Levels of ¹²C and ¹³C forms of metabolic intermediates were monitored with LC-MS/MS. See Munger J, Bennett B D, Parikh A, Feng X J, McArdle J, et al. (2008) Systems-level metabolic flux profiling identifies fatty acid synthesis as a target for antiviral therapy. Nat Biotechnol 26: 1179-1186, which is incorporated herein by reference as if fully set forth.

Metabolite uptake and excretion: Medium was sampled from cells under a variety of conditions: P, CI7, CI14, CI14SS7, SS4, SS7 low glucose/low glutamine P, low glucose/low glutamine CI14. Conditioned medium was sampled over a time course from 0 to 96 hours for fibroblasts depending upon the experiment. The levels of glucose, lactate, glutamine and glutamate were measured using a YSI 7100 Select™ Biochemistry Analyzer (YSI Incorporated, Yellow Springs, Ohio). The rate of glucose consumption, lactate excretion, glutamine consumption and glutamate excretion was determined as the rate that these metabolites appeared or disappeared from the medium divided by the time integral of the protein mass of cells on the plate during that time period.

PPP inhibition and PI live/dead analysis: P and CI14 fibroblasts were treated with dehydroepiandrosterone dissolved in ethanol or dimethylsufoxide (0.1% vol/vol) for four days. On the fourth day of treatment with the inhibitor, cells were trypsinized and collected into conditioned media. Cells were then centrifuged for 5 min at 1000 rpm. The supernatant was aspirated and cells were taken up in PBS with 1 μg/ml PI (VWR, West Chester, Pa.). Cells were kept on ice and immediately analyzed by flow cytometry using a BD LSRII multi-laser analyzer (BD Biosciences, San Jose, Calif.). PI was excited at 488 nm and emitted fluorescence was collected through a 610/20 bandpass filter. At least 40,000 cells were collected and analyzed with FACSDiVa software (BD Biosciences, San Jose, Calif.). PI negative cells were counted as live cells and PI positive cells were counted as dead cells.

PPP inhibition and apoptosis analysis: Apoptosis was measured based on the levels of caspase 3/7 released into the media using the ApoTox-Glo Triplex Assay according to the manufacturer's instructions (Promega Corp., Madison, Wis.). Cells were plated in triplicate at 10,000 cells per well in white-walled, clear-bottom 96-well plates (Costar, Corning Life Sciences, Lowell, Mass.). For contact inhibition, cells were plated 7 days prior to the start of treatment, for serum starvation cells were plated 4 days prior to treatment and switched to 0.1% serum media for the remaining 3 days, while proliferating cells were plated the day prior to the start of treatment. Increasing concentrations of DHEA or ethanol vehicle alone were added to media in each well and treatment proceeded for four days. Cells in serum starvation conditions were incubated in 0.1% serum during treatment as well. The apoptosis reagent was added at 100 μl per well and incubated for 1 h prior to reading. Luminescence was read from the top using a Synergy-2 plate reader (Biotek, Winooski, Vt.). Luminescence data were normalized to the vehicle only condition.

Measurement of carbon incorporation into fatty acids: Lipid synthesis from glutamine was measured using a modified version of a previously published protocol. See Munger J, Bennett B D, Parikh A, Feng X J, McArdle J, et al. (2008) Systems-level metabolic flux profiling identifies fatty acid synthesis as a target for antiviral therapy. Nat Biotechnol 26: 1179-1186, which is incorporated herein by reference as if fully set forth. Briefly, P, CI7, CI14 and CI14SS7 fibroblasts were incubated in medium containing 5 μCi/ml [U-¹⁴C]-glutamine at 4 mM (0.4% labeled). After incubation for 24 h, the culture medium was aspirated, cells were washed with PBS and phospholipids were extracted by addition of 500 μl of 3:2 hexane:isopropanol. The culture dishes were then washed with an additional 500 μl of the hexane:isopropanol mixture. The resulting total extract was dried using a speed-vac, resuspended in 500 μl of 1 N KOH in 90:10 methanol:water and incubated at 70° C. for 60 min to saponify lipids. Sulfuric acid (100 μl, 2.5 M) was then added, followed by hexane (700 μl) to extract the saponified fatty acids. The organic and aqueous phases were separated by centrifugation and scintillation-counted.

Microarray analysis: To monitor gene expression levels, P, CI7 or CI14 fibroblasts were trypsinized, from the plate, pelleted and stored at −80° C. Total RNA was isolated using the mirVana miRNA Isolation kit (Ambion, Austin, Tex.) according to the manufacturer's instructions. RNA quality was verified using a Bioanalyzer 2100 (Agilent Technology, Santa Clara, Calif.) and the amount was determined with a Nanodrop spectrophotometer (NanoDrop Technologies, Wilmington, Del.). 325 ng total RNA was amplified using Low RNA Input Fluorescent Labeling Kit (Agilent Technologies) according to the manufacturer's protocol. Cyanine 3-CTP (Cy-3) (Perkin Elmer, Waltham, Mass.) was directly incorporated into the cRNA from P cells during in vitro transcription. Cyanine 5-CTP (Cy-5) was incorporated into cRNA from CI17 or CI14 fibroblasts. Mixtures of Cy-3 labeled and Cy-5 labeled cRNA were co-hybridized to Whole Human Genome Oligo Microarray slides (Agilent Technologies) at 60° C. for 17 hrs and subsequently washed according to the Agilent standard hybridization protocol. Slides were scanned with a dual laser scanner (Agilent Technologies). Images were monitored for quality control. The Agilent feature extraction software, in conjunction with the Princeton University Microarray database (PUMAdb http://puma.princeton.edu/), was used to compute the log ratio of the two samples for each gene after background subtraction and dye normalization. The entire experiment was performed twice.

Analysis of extracellular matrix protein levels in conditioned medium: For the analysis of extracellular matrix proteins in conditioned medium, the experiments could not be performed in the presence of high amounts of serum because serum inhibited protein transfer after immunoblotting. As previously described, proliferating fibroblasts were conditioned at low cell density in the presence of platelet-derived growth factor with either no serum or 0.1% serum. See Pollina E A, Legesse-Miller A, Haley E M, Goodpaster T, Randolph-Habecker J, et al. (2008) Regulating the angiogenic balance in tissues. Cell Cycle 7: 2056-2070, which is incorporated herein by reference as if fully set forth. Quiescent fibroblasts were cultured at high density in the absence of platelet-derived growth factor with either no serum or 0.1% serum. Medium was conditioned over four days and during that time, protein lysates were collected over a timecourse. The protein content of the cell lysates was plotted against the time of lysate collection. A curve that fit the data was generated and the area under the curve, the integrated protein-hour quantity, was divided by the volume of media collected from the proliferating or quiescent plate. The total protein-hour/volume for each sample was used to adjust the volume of conditioned medium, which was then mixed with 25% volume of trichloroacetic acid (Sigma-Aldrich) containing 0.1% sodium deoxycholate (Sigma-Aldrich), and incubated for thirty minutes on ice. Following centrifugation, samples were washed 3-4 times with −20° C. acetone, resuspended in sodium dodecyl sulfate-polyacrylamide gel electrophoresis sample buffer and separated under reducing conditions on 5% (for fibronectin and COL21A1) or 12% (for LAMA2) sodium dodecyl sulfate-polyacrylamide gels. Proteins were transferred for 1 hour at 100 volts to Westran polyvinylidene fluoride membranes (Perkin Elmer, Waltham, Mass.). Membranes were blocked for 1 hour at room temperature in 5% non-fat dried milk in PBS with 0.1% Tween-20 (PBS-T). Membranes were then incubated overnight at 4° C. with a mouse monoclonal anti-fibronectin clone HFN7.1 (1:2000 dilution, generous gift of Jean Schwarzbauer, Princeton University), mouse polyclonal antibody against COL21A1 (1:750 dilution, Abcam, Cambridge, Mass.), or mouse monoclonal antibody against LAMA2 (3 μg/ml, Abnova, Taipei, Taiwan) diluted in PBS-T/1% milk. Following overnight incubation in the primary antibody, membranes were washed three times in PBS-T, incubated for 1 hour in a 1:10,000 dilution of horseradish peroxidase-conjugated sheep anti-mouse secondary antibody (GE Healthcare) in PBS-T/1% milk. Membranes were exposed to x-ray film, and film was scanned with a Hewlett-Packard Scanjet 4890 using Hewlett-Packard software. The intensity of individual bands was determined with ImageJ analysis software.

Computational determination of fluxes: Fluxes were determined by integration of all available forms of experimental data within a quantitative flux-balanced framework using the same strategy as described in Munger et al. 2008. See Munger J, Bennett B D, Parikh A, Feng X J, McArdle J, et al. (2008) Systems-level metabolic flux profiling identifies fatty acid synthesis as a target for antiviral therapy. Nat Biotechnol 26: 1179-1186, which is incorporated herein by reference as if fully set forth. An ODE model (FIGS. 7A-B) of central carbon metabolism was constructed. The model assumes steady-state, mass-balanced flux and simulates the resulting labeling dynamics after switching cells from unlabeled media to uniformly ¹³C-labeled glucose or glutamine. The model consists of 55 ODEs, describing the rate of loss of unlabeled metabolites and the rate of accumulation of labeled metabolites. It builds upon the previously described model (See Munger J, Bennett B D, Parikh A, Feng X J, McArdle J, et al. (2008) Systems-level metabolic flux profiling identifies fatty acid synthesis as a target for antiviral therapy. Nat Biotechnol 26: 1179-1186, which is incorporated herein by reference as if fully set forth) with a few changes. An exchange flux (F₁₂) was introduced in glycolysis between DHAP and FBP. Backward flux (F₁₁) from α-ketoglutarate to citrate, together with a latent citrate pool that is never labeled (determined by the lowest unlabeled citrate pool size observed in all experiments), was introduced in the TCA cycle. The latent citrate pool was added because for citrate, but not other metabolites, a substantial fraction of the pool (approximately 40% for the proliferating cells) did not label over the course of the experiment. Beyond labeling dynamics, additional input data included metabolite levels, rates of metabolite consumption and excretion, and the glycolysis-PPP flux convergence ratio determined after feeding [1,2-¹³C]-glucose for 2 h. Model parameters (fluxes, as well as pool sizes of a small number of metabolites that could not be directly experimentally measured) were identified by a genetic algorithm that minimizes a cost function defined as the sum of weighted differences between the experimental data and computational results (TABLE 3). See Feng X J, Rabitz H (2004) Optimal identification of biochemical reaction networks. Biophys J 86: 1270-1281, which is incorporated herein by reference as if fully set forth. As a global search algorithm, the genetic algorithm computationally probes for alternative flux solutions consistent with the experimental results. For each cell type, the algorithm was run until 1000 consistent solutions (i.e., parameter sets that produced the lowest cost values when the algorithm reached convergence) were obtained. The distribution of the 1000 values was then used to quantitatively represent each identified parameter. Since the distributions are not Gaussian, a flux is considered quantitatively different between proliferating and quiescent cells only when the distributions from the proliferating and quiescent fibroblasts do not overlap. This measure minimizes the false positives that may occur when only one or a few solutions are identified. Although qualitatively supportive of the model-inferred enhancement of anapleurotic flux from glucose in quiescent fibroblasts, labeling data for [3-¹³C]-glucose, which was taken at 8 hours, were quantitatively inconsistent with the other labeling data, which covered the first two hours of incubation only. The [3-¹³C]-glucose data were accordingly excluded from the computational analysis.

TABLE 3
Functional forms of the components of the cost function for the genetic algorithm.
$The expression for the total cost is J = \frac{1}{5} {\sum }_{n = 1}^{5} {J}_{n} + {J}_{6} .$
The best possible cost value is 1 when the model results fit all experimental data perfectly.

| Description | Equation | Variables |
|---|---|---|
| Kinetic flux profiling | ${J}_{1} = {\sum }_{i = 1}^{{N}_{sp}} {\sum }_{t = 1}^{{N}_{t}} \{ \begin{matrix}1 : \vert {M}_{i , t}^{calc} - {M}_{i , t}^{exp} \vert \leq {ɛ}_{i , t} \\ \frac{\vert {M}_{i , t}^{calc} - {M}_{i , t}^{exp} \vert}{{ɛ}_{i}} : \vert {M}_{i , t}^{calc} - {M}_{i , t}^{exp} \vert > {ɛ}_{i , t}\end{matrix} \}$ | Nₛₚ =Number of species (55, for both glucose and glutamine labeling) Nₜ =Number of experimental time points M_{i,t}^{calc} = calculated value for the i^{th} metabolite at the t^{th} time point M_{i,t}^{exp} = measured laboratory value for the i^{th} metabolite at the t^{th} time point εᵢ = average of 1 SD of the laboratory measurement for the i^{th} metabolite across all time points |
| Uptake and excretion | ${J}_{2} = 8 \times {\sum }_{1}^{4} \{ \begin{matrix}1 : \vert {F}_{i}^{calc} - {F}_{i}^{exp} \vert \leq {ɛ}_{i} \\ \frac{\vert {F}_{i}^{calc} - {F}_{i}^{exp} \vert}{{ɛ}_{i}} : \vert {F}_{i}^{calc} - {F}_{i}^{exp} \vert > {ɛ}_{i}\end{matrix} \}$ | 1: glucose uptake; 2: sum of lactate, alanine, and pyruvate excretion; 3: glutamine uptake; 4: glutamate excretion Fᵢ^{calc} = the calculated value for the i^{th} flux Fᵢ^{exp} = the measured laboratory value for the i^{th} flux εᵢ =estimated laboratory error for the measurement of the i^{th} flux |
| Glycolysis/ PPP ratio | ${J}_{3} = 8 \times \{ \begin{matrix}1 : \vert {R}^{calc} - {R}^{exp} \vert \leq ɛ \\ \frac{\vert {R}^{calc} - {R}^{exp} \vert}{ɛ} : \vert {R}^{calc} - {R}^{exp} \vert > ɛ\end{matrix} \}$ | ${R}^{cal} = \frac{3 {F}_{3}}{{F}_{3} + {F}_{4}}$ ${R}^{exp} = \frac{lactate\_with \_ 1 \_ {}^{13} C\_atom}{lactate\_with \_ 1 \_or \_ 2 \_ {}^{13} C\_atoms}$ |
| | | after 1,2-¹³C-glucose feeding |
| | | ε =estimated laboratory error |
| Protein Synthesis | ${J}_{4} = 8 \times \{ \begin{matrix}1 : X - {X}_{max} \leq ɛ \\ \frac{X - {X}_{max}}{ɛ} : X - {X}_{max} > ɛ\end{matrix} \}$ | X: calculated protein synthesis rate Xₘₐₓ = 30 nMoles/min for proliferating cells and 8 nMoles/min for IC7, IC14, and IC14557 cells |
| Hexose consumption | ${J}_{5} = 8 \times \{ \begin{matrix}1 : {G}^{calc} - {G}^{max} \leq ɛ \\ \frac{{G}^{calc} - {G}^{max}}{ɛ} : {G}^{calc} - {G}^{max} > ɛ\end{matrix} \}$ | G: calculated hexose phosphate outflux G^{max} = 0.2 × glucose uptake rate |
| Penalty for negative fluxes | ${J}_{6} = {\sum }_{i = 1}^{5} \{ \begin{matrix}0.5 : {N}_{i}^{calc} < 0 \\ 0 : {N}_{i}^{calc} \geq 0\end{matrix} \}$ | Nᵢ^{calc} = calculated net flux value |

### Example 20

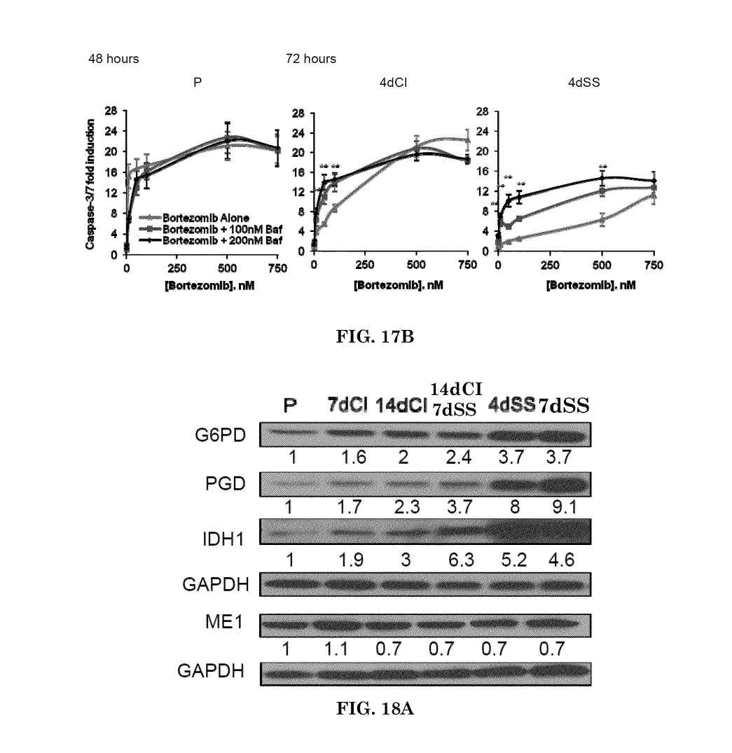

Referring to FIG. 17, the effect of treatment using DHEA and/or an autophagy inhibitor combined with proteasome inhibitors on apoptosis in quiescent cells was analyzed. Referring to FIG. 17A, the combination of DHEA and a proteasome inhibitor was shown to significantly increase the induction of apoptosis in four day serum starved fibroblasts (FIG. 17A). The proteasome inhibitor alone did not result in apoptotic induction in the four day serum starved fibroblasts used in this experiment. However, when varying concentrations of bortezomib were combined with 425 uM of DHEA, it resulted in up to a nearly 17-fold increase in the induction of apoptosis depending on the concentration of proteasome inhibitor utilized.

In this experiment, the proteasome inhibitor, bortezomib, was used in varying concentrations with DHEA during a 48 hour treatment. When DHEA alone was used, there was approximately a 2-fold increase in apoptosis induction. When DHEA was used with bortezomib at a concentration of 0.1 nM, there was a 3.1-fold increase in apoptosis induction. When DHEA was used with bortezomib at a concentration of 10 nM, there was a 1.6-fold increase in apoptosis induction. When DHEA was used with bortezomib at a concentration of 50 nM, there was a 12.7-fold increase in apoptosis induction. When DHEA was used with bortezomib at a concentration of 100 nM, there was a 14.5-fold increase in apoptosis induction. When DHEA was used with bortezomib at a concentration of 500 nM, there was a 15.7-fold increase in apoptosis induction. When DHEA was used with bortezomib at a concentration of 750 nM, there was a 16.3-fold increase in apoptosis induction. Finally, when DHEA was used with bortezomib at a concentration of 1000 nM, there was 16.8-fold increase in apoptosis induction.

Other combinations have also been shown to potentiate apoptosis. For example, the combination of the autophagy inhibitor bafilomycin, DHEA, and bortezomib has also been used to potentiate apoptosis in quiescent cells. Different proteasome inhibitors combined with DHEA have also shown the ability to increase the induction of apoptosis in quiescent cells. Specifically, the combination of DHEA and the proteasome inhibitor, MG132, has been used to induce apoptosis.

Combining an inhibitor of autophagy with a proteasome inhibitor may also potentiate apoptosis. For example, the combination bafilomycin and MG132 has been shown to increase the induction of apoptosis in quiescent cells.

Any of the examples and embodiments herein may be modified by providing a proteasome inhibitor with a at least one of a PPP inhibitor or an authophagy inhibitor.

### Example 21

### Novel Quiescent Fibroblast NADPH Production Pathway

Quiescent, serum-starved fibroblasts activate a program of increased NADPH production that results in an increase in the levels of reduced glutathione and protects quiescent fibroblasts from the accumulation of oxidized proteins and apoptosis.

Quiescent fibroblasts induce a program of NADPH generation

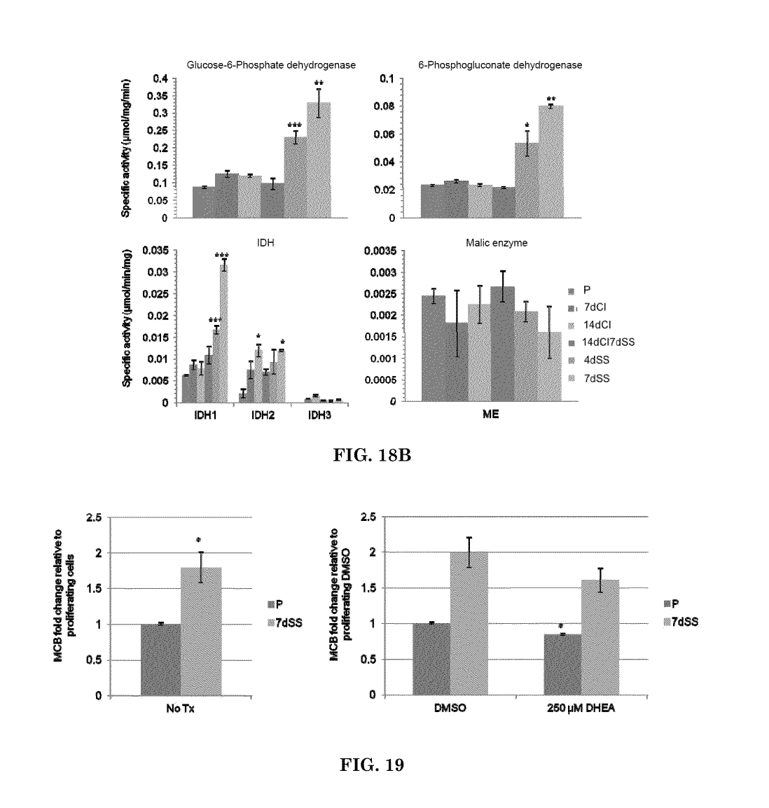

Referring to FIGS. 18A and 18B, NADPH production is induced in quiescent fibroblasts. G6PD, PGD and IDH1 are expressed at low levels in proliferating fibroblasts, at higher levels in 7 dCI, 14 dCI and 14 dCI7 dSS fibroblasts, and at even higher levels in 4 dSS and 7 dSS fibroblasts (FIGS. 18A and 18B). Malic enzyme, another protein that generates NADPH but was not predicted to be differentially active in proliferating versus quiescent fibroblasts, was expressed at similar levels in cells in all conditions. The activity of the NADPH-producing enzymes in cytosolic or mitochondrial lysates collected from cells in different proliferative conditions is shown in FIG. 18A. Immunoblotting confirmed successful separation of mitochondrial and cytosolic proteins. Lysates were incubated with the appropriate substrate and the rate of enzymatic activity was determined based on the appearance or disappearance of NADH or NADPH. G6PD had the highest specific activity among the enzymes monitored, consistent with previous reports that it is the most important contributor to NAPDH levels. G6PD activity was significantly higher in 4 dSS and 7 dSS fibroblasts and somewhat elevated in 7 dCI and 14 dCI fibroblasts compared to proliferating fibroblasts. PGD activity was significantly elevated in 4 dSS and 7 dSS fibroblasts. Thus NADPH-generating enzymes in the pentose-phosphate pathway are activated in serum-starved fibroblasts.

The activity of cytoplasmic NADP-dependent IDH1, mitochondrial NADP-dependent IDH2 and mitochondrial NAD-dependent isocitrate dehydrogenase 3 (IDH3) were monitored. Enzymatic activity assays performed on mitochondrial and cytosolic lysates revealed that IDH1 had higher activity in 7 dCI, 14 dCI and 14 dCI7 dSS states, and significantly elevated activity in 4 dSS and 7 dSS fibroblasts (FIG. 18B). IDH2 exhibited higher activity in quiescent than proliferating fibroblasts with comparable activity levels in all quiescent conditions tested. The activity of IDH3 was very low and at the limit of detection of the assays. Low levels of IDH activity in proliferating fibroblasts is consistent with the previous observation that proliferating fibroblasts exhibit little flux from isocitrate to α-ketoglutarate. In proliferating fibroblasts, citrate may be transported to the cytosol and acetyl CoA derived from citrate may be used for lipid biosynthesis or for the acetylation of proteins or histones. In quiescent fibroblasts, in contrast, there is significant flux from isocitrate to α-ketoglutarate, and we show here that quiescent fibroblasts rely on the NADP-dependent isocitrate dehydrogenases (IDH1 and IDH2) to fuel TCA cycle flux preferentially over the canonical NAD-dependent isozyme (IDH3). Further, the most active enzyme is cytoplasmic IDH1, which suggests that citrate is shuttled out of the mitochondria to the cytoplasm and α-ketoglutarate may be shuttled back from the cytoplasm to the mitochondria. Favored use of the cytoplasmic IDH1 enzyme to convert isocitrate to α-ketoglutarate could reflect a cellular drive for cytosolic NADPH. The activity of NADP-dependent malic enzyme did not change significantly across the different states, indicating that this enzyme is not part of the NADPH-producing program of quiescent fibroblasts. Thus, four different enzymes were identified, all of which generate NADPH, that exhibit elevated activity in serum-starved fibroblasts compared with proliferating fibroblasts.

Glutathione reductase activity is increased in serum-starved fibroblasts

NADPH is an important co-factor in biosynthetic reactions like fatty acid biosynthesis, and it can also be used to maintain redox homeostasis as a cofactor for the conversion of oxidized to reduced glutathione by glutathione reductase. Both fatty acid synthase (FASN) and glutathione reductase (GR) were expressed at higher levels in contact-inhibited fibroblasts and at even higher levels in serum-starved fibroblasts compared to proliferating fibroblasts. In terms of enzymatic activity, fatty acid synthase activity was lower in contact-inhibited fibroblasts than in proliferating fibroblasts and significantly higher in serum-starved fibroblasts. Serum-starved fibroblasts may upregulate fatty acid synthase as a response to the lack of fatty acids in serum. Glutathione reductase activity was higher in contact-inhibited fibroblasts than in proliferating fibroblasts and significantly higher in serum-starved fibroblasts. The high specific activity of glutathione reductase suggests that regeneration of reduced glutathione may be an important function of the NADPH production program in serum-starved fibroblasts.

Serum-starved fibroblasts contain higher levels of intracellular reduced glutathione (FIG. 19)

It was tested whether the increase in glutathione reductase activity in serum-starved fibroblasts was associated with elevated levels of reduced glutathione. 7 dSS fibroblasts were focused on because they had the highest activity of the four enzymes in the NADPH production pathway. Flow cytometry was used to monitor the levels of glutathione in proliferating and 7 dSS fibroblasts with monochlorobimane (MCB), a compound that forms blue fluorescent adducts when it reacts with intracellular reduced glutathione (Sebastia et al., “Evaluation of fluorescent dyes for measuring intracellular glutathione content in primary cultures of human neurons and beuroblastoma SH-SY5Y, Cytometry A 51, 16-25, 2003, which is incorporated herein by reference as if fully set forth). Serum-starved fibroblasts contained significantly higher levels of reduced glutathione than proliferating fibroblasts (FIG. 19). The results are consistent with a model in which the NADPH production program activated in the serum-starved fibroblasts contributes to a larger pool of reduced glutathione.

G6PD inhibitors deplete NADPH levels

The functional effects of NADPH production were tested by treating cells with an uncompetitive inhibitor of G6PD, 5-dehydroepiandrosterone (DHEA) (Shantz et al., “Mechanism of Inhibition of Growth opf 3T3-L1 fibroblasts and their differentiation to adipocytes by dehydroepiandrosterone and related steroids: role of glucose-6-phosphate dehydrogenase,” Proc Natl Acad Sci USA 86, 3852-3856, 1989, which is incorporated herein by reference as if fully set forth). Serum-starved fibroblasts are particularly sensitive to DHEA-induced apoptosis based on increased caspase-3/7 activity (FIG. 12B). These results are consistent with a more important role for pentose phosphate pathway-based NADPH production in serum-starved fibroblasts than for cells in other states. Further, the ability to selectively induce apoptosis in serum-starved fibroblasts is potentially clinically valuable because most of the agents currently available for inducing apoptosis target actively proliferating cells (Barnes and Melo, “Primitive, quiescent and difficult to kill: the role of the non-proliferating stem cells in chronic myeloid leukemia,” Cell Cycle 5, 2862-2866, 2006, which is incorporated herein by reference as if fully set forth). To ensure that DHEA treatment had the anticipated effects, and that cells in all conditions are dosed similarly, it was confirmed that G6PD activity declined in proliferating cells, and significantly declined in serum-starved fibroblasts treated with DHEA.

Glutathione Depletion Correlates with Apoptosis in Serum-Starved Fibroblasts

It was expected that the reduction in NADPH levels that resulted from DHEA treatment would result in lower activity of glutathione reductase and thus a decrease in the levels of reduced glutathione. Using flow cytometry, smaller pools of intracellular GSH were detected in serum-starved fibroblasts after DHEA treatment (FIG. 19). The depletion of glutathione in response to DHEA could be an important part of the apoptotic pathway. Alternatively, because apoptosing cells can rapidly and selectively release glutathione to the extracellular environment (van den Dobbelsteen et al., 1996, “Rapid and specific efflux of reduced glutathione during apoptosis induced by anti-Fas/APO-1 antibody,” J Biol Chem 271, 15240-15427; Franco et al., “Glutathione depletion is necessary for apoptosis in lymphoid cells independent of reactive oxygen species,” J Biol Chem 282, 30452-30465, 2007, which are incorporated herein by reference as if fully set forth), the depletion of glutathione could represent a response to a distinct apoptotic trigger. Mass spectrometry was used to measure glutathione levels in conditioned medium from 7 dSS fibroblasts treated with DMSO as a control or with DHEA. There was no increase in extracellular glutathione in conditioned medium from DHEA-treated fibroblasts compared with controls; in fact, levels were lower, consistent with a DHEA-induced reduction in intracellular levels of reduced glutathione. Thus, the decrease in the levels of reduced glutathione in DHEA-treated 7 dSS fibroblasts is not because glutathione is excreted from the cells. Taken as a whole, the data are consistent with serum-starved fibroblasts containing higher glutathione pools that are important for the maintenance of their viability, as their depletion with DHEA results in apoptosis.

Treatment with DHEA Results in Increased Oxidized Proteins in Serum-Starved Fibroblasts

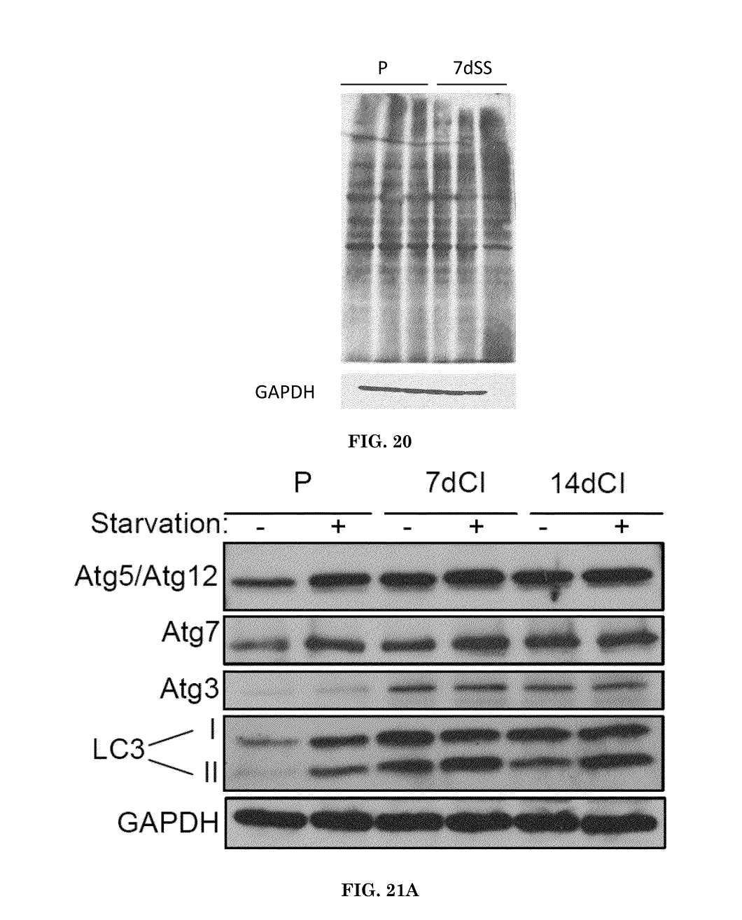

Glutathione plays an important role in ROS scavenging, both by acting as a cofactor for glutathione peroxidase and via direct interaction with ROS (Jones et al., “Kinetics of superoxide scavenging by glutathione: an evaluation of its role in the removal of mitochrondrial superoxide, Biochem Soc Trans 31, 1337-1339, 2003, which is incorporated herein by reference as if fully set forth). The effects of DHEA treatment on the levels of oxidized proteins were monitored in proliferating and 7 dSS fibroblasts (FIG. 20). Oxidized, carbonylated proteins were derivatized with 2, 4 dinitrophenol and monitored with an anti-2,4, dinitrophenol antibody. DHEA treatment had little effect on oxidized protein levels in proliferating fibroblasts, but significantly increased the levels of oxidized protein in 7 dSS fibroblasts (FIG. 20). Thus, the 7 dSS fibroblasts rely on DHEA-sensitive pathways to maintain low levels of oxidized protein.

### Example 22

### Autophagy Induction in Quiescent Dermal Fibroblasts Promotes the Degradation of Oxidized and Nitrosylated Proteins

Autophagy is induced in contact-inhibited human fibroblasts despite the presence of full nutrients.

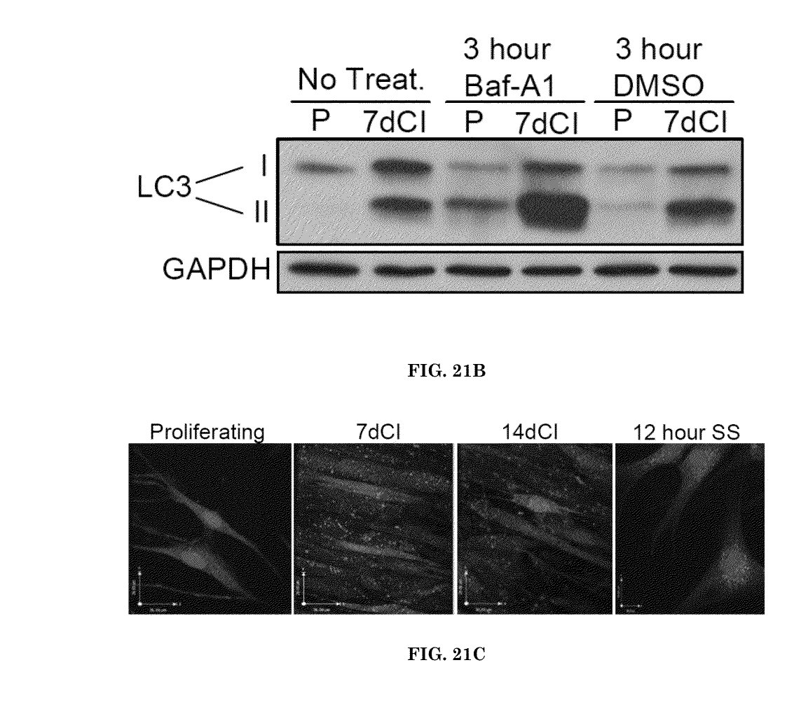

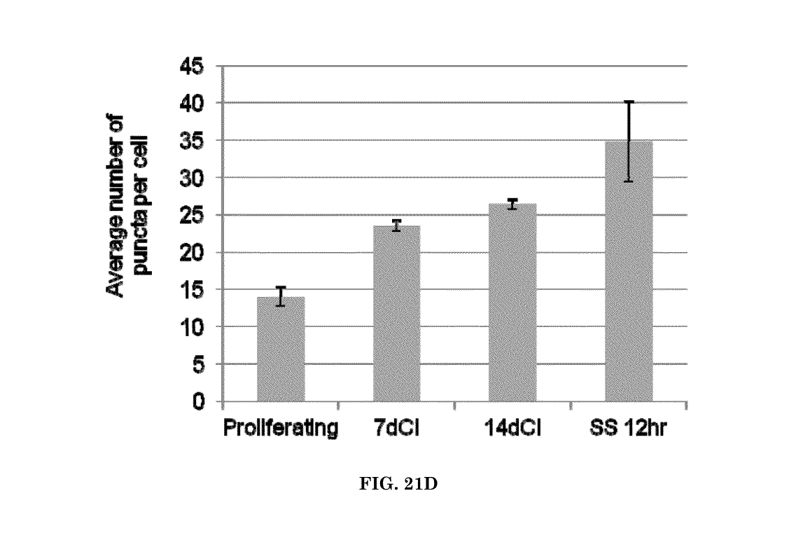

The levels of autophagy components—Atg5/Atg12, Atg7, Atg3, and LC3-I and LC3-II—were monitored in proliferating and contact-inhibited fibroblasts using immunoblotting (FIG. 21A). As a positive control for autophagy induction, samples of cells in each cell cycle state were also incubated in Kreb's Ringer Bicarbonate solution (KRB), a protocol that induces autophagy through amino acid starvation and elimination of other nutrients except glucose. Levels of Atg5/Atg12, Atg7, and LC3-II were elevated in all of the amino acid starvation and contact-inhibition states as compared to the proliferating state (FIG. 21A). Atg3 was induced in all quiescent states compared with proliferating states, including amino acid starvation. In order to differentiate whether the high levels of autophagy proteins resulted from a blockade of autophagy degradation or active flux through the pathway, we monitored the levels of LC3-II in proliferating and 7 dCI fibroblasts in the presence of bafilomycin A1 (Baf-A1). Baf-A1 is a vacuolar-type H⁺-ATPase inhibitor that prevents autophagosome fusion with the lysosome due to an increase in lysosomal pH. Baf-A1 treatment resulted in a further increase in the levels of LC3-II and a protein degraded by autophagy, p62/SQSTM1 (p62), in contact-inhibited fibroblasts as compared to proliferating or contact-inhibited vehicle-treated or non-treated controls (FIG. 21B). This finding is consistent with active autophagic flux in quiescent fibroblasts

To more directly assay autophagosome formation in contact-inhibited fibroblasts, confocal microscopy of fibroblasts stably expressing a retrovirally-encoded GFP-LC3 fusion protein were used. GFP-positive punctate structures, which represent autophagosomes, were visualized in proliferating and quiescent cells in culture. Contact-inhibited fibroblasts (7 dCI and 14 dCI) contained significantly more autophagic puncta than proliferating cells as measured by quantifying the number of GFP-positive puncta per cell in each cell cycle condition (FIGS. 21C and 21D). An average number of puncta of 14±1.3 was found in proliferating fibroblasts (n=45), 23.5±0.71 in 7 dCI cells (n=153; p=0.0002), and 26.4±0.7 for 14 dCI samples (n=191; p=6.13×10⁻⁶) (FIGS. 21C and 21D). Puncta levels in cells that were deprived of serum were also monitored, because serum starvation is a known strong signal for autophagy induction. The mean number of puncta in the serum-starved cells (n=10) was 34.8±5.4, which was higher than, but comparable to, the number of puncta in the contact-inhibited fibroblasts. These findings support our conclusion that entry into quiescence is associated with an induction of autophagy at a level sufficient to have functional consequences for the cell.

Autophagy Limits Oxidized and Nitrosylated Protein Accumulation in Quiescent Fibroblasts.

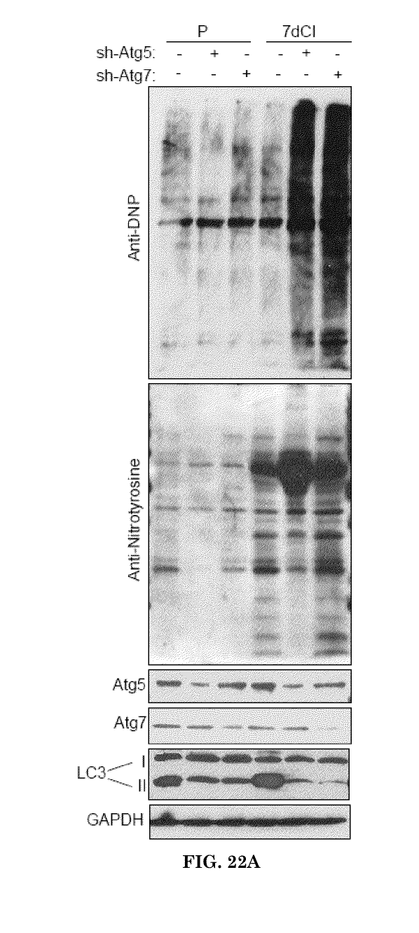

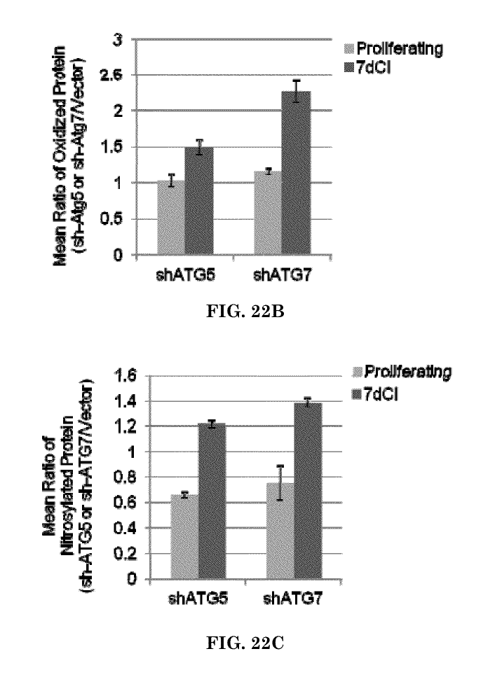

In addition to its role in providing amino acids and energy under starvation conditions, autophagy is involved in maintenance of cellular homeostasis and resistance to tumorigenesis through degradation of old or damaged proteins and entire organelles. It was hypothesized that autophagy in contact-inhibited cells could function to degrade old and/or damaged proteins that would otherwise accumulate in the cytoplasm due to lack of cell division as a mechanism for dilution of these proteins. Oxidation of proteins causes the formation of carbonyl groups on amino acids, and carbonylation can disrupt protein function. To monitor the extent of protein oxidation in proliferating and contact-inhibited fibroblasts, the protein carbonyl groups were derivatized using 2,4-dinitrophenylhydrazine (DNP), and monitored using immunoblotting with an antibody that recognizes the covalently added DNP. The levels of oxidized proteins in proliferating and 7 dCI fibroblasts were monitored in cells that were either competent to perform autophagy (control), or were stably expressing an shRNA against the essential autophagy components Atg5 (sh-Atg5) or Atg7 (sh-Atg7), which are known to represent autophagy-defective phenotypes in other model systems. In sh-Atg5 and sh-Atg7 fibroblasts, protein levels of the shRNA target were downregulated, and autophagy levels as measured by LC3-II protein levels, were lower than in cells expressing a control shRNA in both the proliferative and quiescent states (FIG. 22A). In proliferating fibroblasts, introduction of an shRNA against Atg5 or Atg7 did not result in a significant accumulation of oxidized protein. In contact-inhibited fibroblasts, in contrast, the levels of oxidized proteins increased 1.5-fold±0.1 in 7 dCI sh-Atg5 fibroblasts as compared to vector control counterparts (p=0.009) (FIG. 22B). Even more strikingly, we discovered a 2.3-fold±0.2 increase in oxidized protein in 7 dCI sh-Atg7 fibroblasts as compared to vector control counterparts (p=0.0001) (FIG. 22B).

In addition to carbonylation, proteins can also be damaged by nitrosylation reactions that occur secondary to an accumulation of reactive nitrogen species. We monitored nitrosylation of tyrosine residues in proliferating and contact-inhibited fibroblasts proficient and deficient for autophagy by immunoblotting with an antibody that recognizes nitrotyrosine (FIG. 22A). Proliferating control and autophagy-deficient fibroblasts contained similar levels of nitrotyrosine modifications. In contrast, 7 dCI sh-Atg5 fibroblasts contained a 1.2-fold±0.02 increased level of nitrosylated protein (p=0.0001) (FIG. 22C). Similarly, 7 dCI sh-Atg7 fibroblasts contained a 1.38-fold±0.03 higher level of nitrosylation in the collected protein lysates (p=0.009) (FIG. 22C). These findings demonstrate that autophagy may represent a mechanism by which quiescent fibroblasts protect themselves from the potentially damaging effects of reactive oxygen and nitrogen species.

Autophagy in Quiescent Fibroblasts Declines as they Proliferate to Heal Wounds

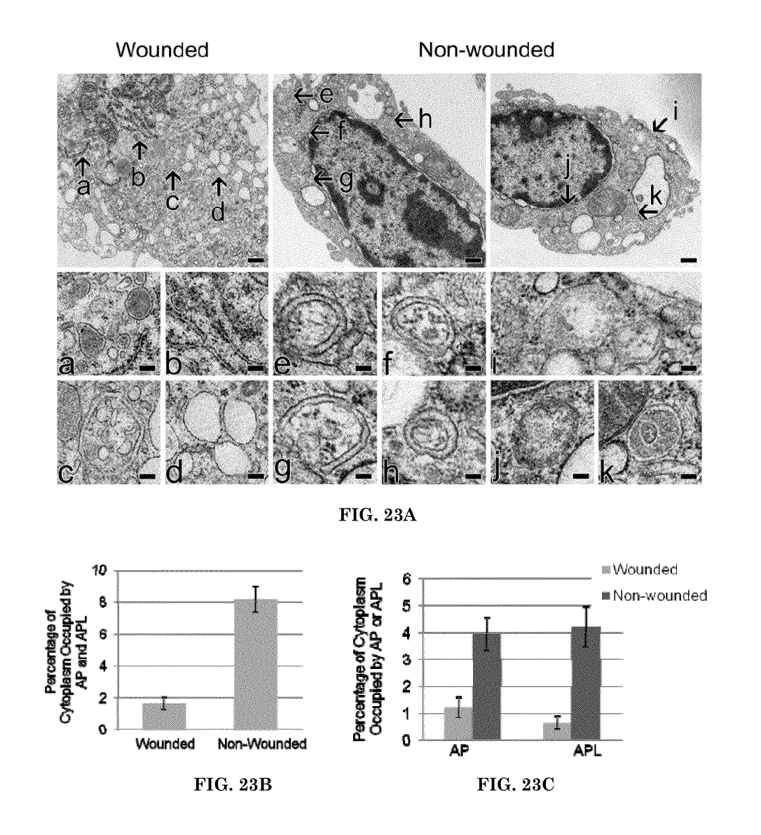

The association between quiescence and autophagy in dermal fibroblasts was addressed in mice. The flanks of three C57BL/6 mice were wounded, and the dermal fibroblasts were monitored using transmission electron microscopy (TEM) on samples collected 24 hours after the induction of the wound. Undisturbed dermal fibroblasts have been shown to be in a quiescent state in vivo. Fibroblasts from non-wounded mouse skin samples were examined as an example of a quiescent cell population and fibroblasts in the wounded area as an example of a proliferative population. Qualitative characterization of fibroblasts from wounded and non-wounded areas of tissue samples was performed (FIG. 23A). The representative fibroblast shown from the skin of a wounded mouse (FIG. 23A, first row of images on the left) contained more developed lysosomes (FIG. 23A a), rough endoplasmic reticulum (FIG. 23A b), and secretory vesicles (FIG. 23A d). In proliferating fibroblasts from wounded tissue, autophagosomes (FIG. 23A c) were present but rare. Fibroblasts from non-wounded areas of the skin (FIG. 23A, middle and right of the first row of images) contained significantly more autophagosomes (FIG. 23A e, f, and h), developing autophagosomes (FIG. 23A g), and autophagolysosomes in different stages of the process of losing their double membrane than fibroblasts from the wounded area (FIG. 23A i-k). A modified grid counting technique was used to determine the percentage of the cytoplasmic area occupied by autophagosomes and autophagolysosomes in fibroblasts from wounded and non-wounded skin (FIG. 23B). For quiescent fibroblasts from non-wound associated areas of mouse skin, 8.2%±0.8% of the cytoplasmic area was occupied by autophagosomes and autophagolysosomes. In contrast, in fibroblasts isolated from mouse skin 24 hours after wounding, 1.6%±0.4% of the cytoplasmic area was occupied by these vesicles (p=2.95×10⁻⁸). Autophagosomes and autophagolysosomes were separately quantified (FIG. 23C), and determined that the ratio of autophagosomes to autophagolysosomes (AP:APL) is 2.7 in wounded fibroblasts and 0.93 in non-wounded cells. Thus, there were almost three times more autophagosomes than autophagolysosomes in proliferating cells, suggesting that autophagosomes might not be efficiently transported to the lysosome for degradation in proliferating cells. A ratio close to one in the quiescent cells in the non-wounded skin is consistent with autophagosomes being trafficked to lysosomes, rather than accumulating in the cytoplasm. Quiescent dermal fibroblasts within skin have elevated levels of autophagy as compared to proliferative fibroblasts in wounded skin. Quiescent fibroblasts both in vitro and in vivo are characterized by the autophagy pathway and entry into the proliferative cell cycle results in loss of autophagic vesicles.

### Example 23

### Quiescent Fibroblasts are Protected from Proteasome Inhibition-Mediated Toxicity

Quiescent fibroblasts are less sensitive than proliferating cells to proteasome inhibition.

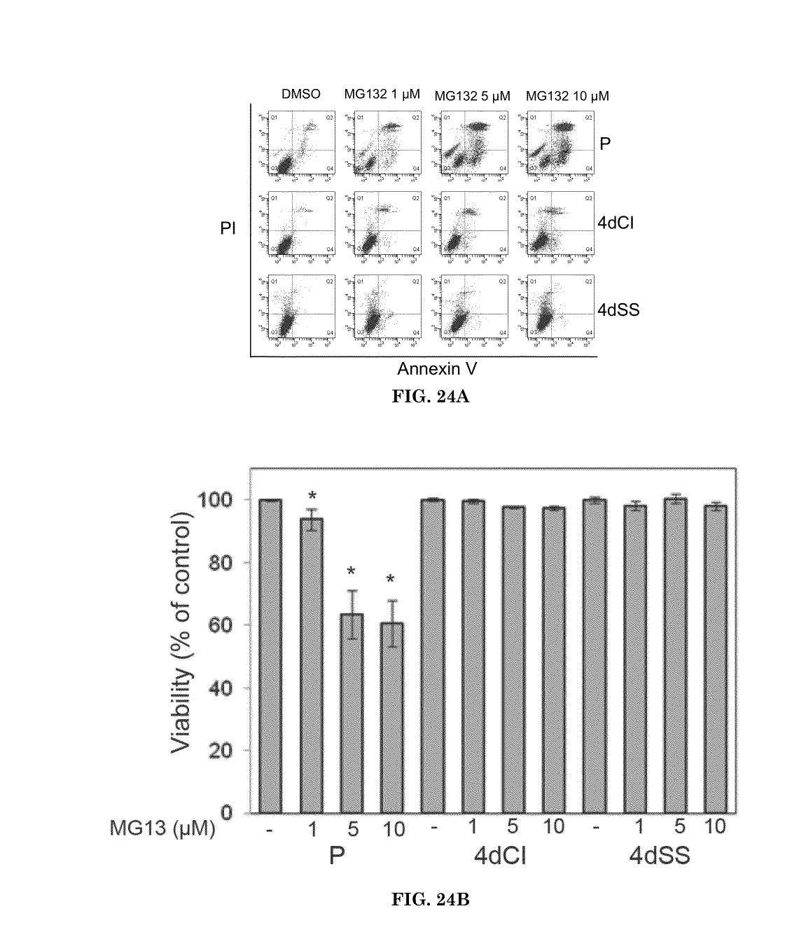

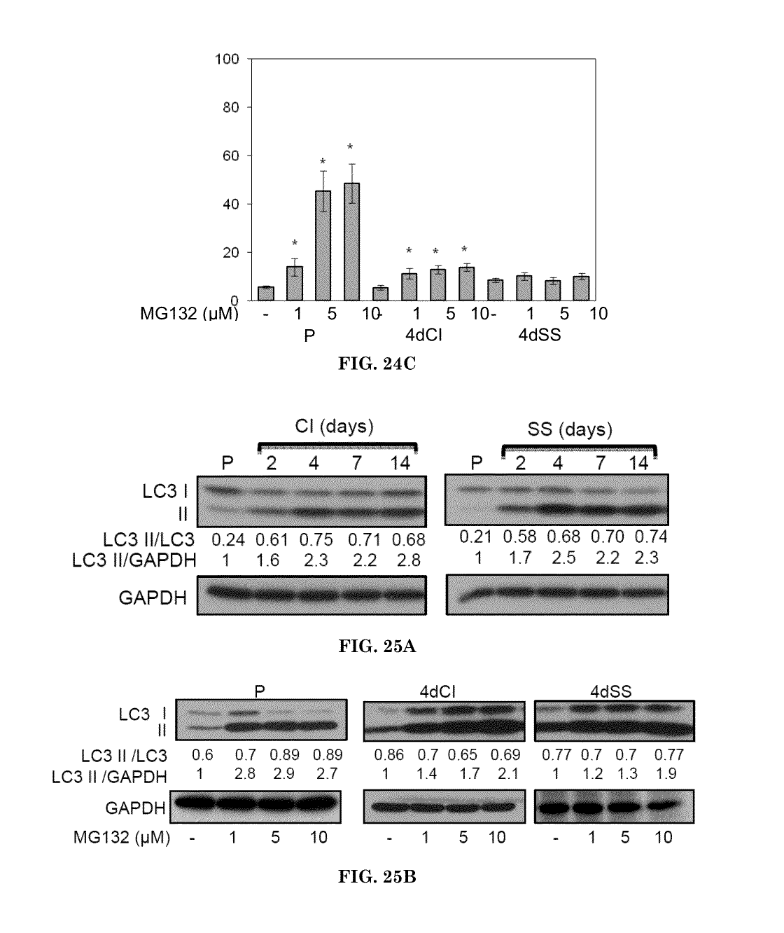

To elucidate the responses of proliferating and quiescent fibroblasts to proteasome inhibition, the effects of known proteasome inhibitors on proliferating and quiescent fibroblasts were examined. The induction of apoptosis and cell death was monitored using the apoptotic marker annexin V and the cell viability marker PI. A representative flow scatter plot is presented in FIG. 24A. Treatment with either MG132 or epoxomicin for 24 hours resulted in a statistically significant, dose-dependent decrease in proliferating cell viability based on the fraction of cells negative for PI (FIG. 24B). In contrast, quiescent cells maintained high viability at 24 hours even at concentrations of proteasome inhibitors that reduced the viability of proliferating cells by almost 50% (10 μM for MG132, 1 μM for epoxomicin) (FIG. 24B).

After 24 hours of treatment with MG132, proliferating cells exhibited a significant increase in annexin V and PI staining. At the highest dose (10 μM), approximately 50% of proliferating cells were apoptotic (FIG. 24A-lower right (Q4), upper right (Q2), and upper left (Q1) quadrants represent early apoptosis, late apoptosis, and very late apoptosis or necrosis, respectively). In comparison, quiescent fibroblasts were largely unaffected by MG132 treatment, showing far lower levels of apoptosis. At the highest dose of MG132, approximately 14% of the contact-inhibited fibroblasts and 10% of the serum-starved fibroblasts exhibited signs of apoptosis (FIG. 24C). Even after 48 hours of MG132 treatment, a significantly higher number of quiescent fibroblasts maintained viability than proliferating fibroblasts.

Proliferating and Quiescent Fibroblasts Induce Autophagy in Response to Proteasome Inhibition

The mechanisms by which quiescent fibroblasts remain viable despite proteasome inhibition were sought. Several studies have reported that autophagy serves as a survival mechanism in cells treated with proteasome inhibitors (Milani et al., 2009, “The role of ATF4 stabilization and autophagy in resistance of breast cancer cells treated with Bortezomib, Cancer Res 69, 4415-4423, which is incorporated herein by reference as if fully set forth) and that autophagy is induced in both serum-starved and contact-inhibited quiescent cells (Valentin and Yang, 2008, “Autophagy is activated, but is not required for the G0 function of BCL-2 or BCL-xL,” Cell Cycle, 7, 2762-2768, which is incorporated herein by reference as if fully set forth). It was hypothesized that autophagy might play a role in protecting quiescent fibroblasts from proteasome inhibition-mediated cell death.

To test this hypothesis, the levels of an autophagy-specific form of the LC3 protein, LC3 II, were monitored compared to a housekeeping protein (Klionsky et al., 2008, “Guidelines for the use and interpretation of assays for monitoring autophagy in higher eukaryotes,” Autophagy 4, 151-175, which is incorporated herein by reference as if fully set forth). A time-dependent increase in the ratio of LC3 II to GAPDH was observed as cells were induced into quiescence by serum starvation or contact inhibition (FIG. 25A). Consistent with published data, these results confirm the induction of autophagy in both contact-inhibited and serum-starved primary fibroblasts (Valentin and Yang, 2008, “Autophagy is activated, but is not required for the G0 function of BCL-2 or BCL-xL,” Cell Cycle, 7, 2762-2768, which is incorporated herein by reference as if fully set forth).

Although proliferating fibroblasts exhibit low baseline levels of autophagy, previous studies suggested that autophagy can be induced in response to proteasome inhibition (Zhu et al., 2010, “Proteasome inhibitors activate autophagy as a cytoprotective response in human prostate cancer cells,” Oncogene 29, 451-462; Kawaguchi et al., 2011, “Combined treatment with bortezomib plus bafilomycin A1 enhances the cytocidal effect and induces endoplasmic reticulum stress in U266 myeloma cells: crosstalk among proteasome, autophagy-lysosome and ER stress,” Int. J. Oncol 38, 643-654, which are incorporated herein by reference as if fully set forth). We observed an increase in the ratio of LC3 II to GAPDH in response to MG132 treatment for cells in proliferating, contact-inhibited and serum-starved states (FIG. 25B). To test whether the high levels of LC3 II in quiescent cells in response to MG132 treatment result from a blockade of autophagy degradation or from active flux through the pathway, LC3 II levels were monitored in proliferating and quiescent fibroblasts treated with MG132 in the presence or absence of bafilomycin A1 (Baf). Baf is a vacuolar-type H(+)-ATPase pump inhibitor that prevents lysosomal-mediated degradation. Baf treatment also blocks the fusion of autophagosomes with lysosomes (Yoshimori et al., 1991, “Bafilomycin A1, a specific inhibitor of vacuolar-type H(+)-ATPase, inhibits acidification and protein degredation in lysosomes of cultured cells,” J Biol Chem 266, 17701-17712; Yamamoto et al., 1998, “Bafilomycin A1 prevents maturation of autophagic vacuoles by inhibiting fusion between autophagosomes and lysosomes in rat hepatoma cell lines, H-4-II-E cells, Cell Struct Funct 23, 33-42, which are incorporated herein by reference as if fully set forth) due to an increase in the lysosomal pH (Yoshimori et al., 1991), which inhibits autophagy (Yamamoto et al., 1998). Baf-treated proliferating and quiescent cells exhibited an increase in the ratio of LC3 II to GAPDH. For proliferating and contact-inhibited cells, Baf treatment in conjunction with MG132 treatment led to a further increase in the LC3 II to GAPDH ratio compared with MG132 treatment alone (FIG. 25C). In comparison, in serum-starved cells Baf treatment combined with MG132 treatment did not change the LC3 II to GAPDH ratio compared with MG132 treatment alone. These results indicate that MG132 treatment results in active autophagic flux in proliferating and contact-inhibited primary fibroblasts (FIG. 25C).

Bafilomycin A1 Sensitizes Serum-Starved Fibroblasts to Proteasome Inhibition-Mediated Apoptosis

To evaluate further the functional role of the autophagy/lysosomal pathway in response to proteasome inhibition in proliferating and quiescent cells, the effect of Baf on proteasome inhibition-mediated induction of apoptosis was monitored. Apoptosis induction was assessed by monitoring caspase 3/7 activity using a luminescent caspase substrate. This assay is optimized for high-throughput screening in 96-well plates and allows multiple concentrations and combination of drugs to be tested in triplicate at different time points. Due to the large number of cells within each well of contact-inhibited cells, a significant increase in caspase 3/7 activity may only represent a small fraction of all of the cells within that well, and thus the data for contact-inhibited cells are difficult to interpret. Therefore, this assay was used to compare proliferating and serum-starved cells only. Changes in caspase 3/7 activity were examined in proliferating and serum-starved cells treated with increasing concentrations of MG132 (0 to 10 μM) in the presence or absence of 100 nM Baf. MG132 treatment resulted in dose-dependent increases in caspase 3/7 activity (FIG. 25D, the graph grey bar is MG132 alone, and the graph black bar is MG132 with bafilomycin), thereby indicating the induction of apoptosis. Baf treatment strongly enhanced MG132-induced apoptosis in serum-starved fibroblasts, but had no significant effect on proliferating fibroblasts at 48 hours post treatment. A slight reduction in caspase 3/7 activity was observed in proliferating cells at 24 hours post treatment. The induction of apoptosis in serum-starved fibroblasts at 48 hours post treatment with 10 μM MG132 more than doubled in the presence of Baf. Similar results were also observed for bortezomib, a potent and specific proteasome inhibitor that is clinically approved for the treatment of multiple myeloma (Hideshima et al., 2001, “The proteasome inhibitor PS-341 inhibits growth, induces apoptosis, and overcomes drug resistance in himan multiple myeloma cells, Cancer Res 61, 3071-3076, which is incorporated herein by reference as if fully set forth), mantle cell lymphoma (Goy et al., 2005, “Phase II study of proteasome inhibitor bortezomib in relapsed or refractory B-cell non-Hodgkin's lymphoma,” J Clin Oncol 23, 667-675, which is incorporated herein by reference as if fully set forth) and other solid tumors. As observed for the MG132 treatment (FIG. 25D), the induction of apoptosis was relatively low in serum-starved fibroblasts compared to proliferating fibroblasts treated with bortezomib. However, after 72 hours of treatment, the proliferating cells were dead, and the combination of bortezomib and Baf had a strong synergistic apoptotic effect in serum-starved cells. The treatment of proliferating fibroblasts with Baf led to reduced bortezomib-induced caspase 3/7 activity at 24 hours post treatment but had little effect after 48 hours. Thus, using two proteasome inhibitors, MG132 and bortezomib, Baf was shown to have a strong, synergistic apoptotic effect on serum-starved fibroblasts.

To further assess the functional role of autophagy with respect to the viability of proteasome-inhibited quiescent cells, proliferating and quiescent fibroblasts were transduced with a retroviral vector containing an shRNA against beclin-1, a critical upstream regulator of autophagy responsible for mediating the initial stages of autophagosome formation (Cao and Klionsky, 2007, “Physiological functions of Atg6/Beclin 1: a unique autophagy-related protein,” Cell Res 17, 839-849, which is incorporated herein by reference as if fully set forth). Immunoblot analysis confirmed beclin-1 depletion of >80% in shbeclin-1-transduced fibroblasts in all cell states. Based on caspase 3/7 activity, beclin-1 knockdown resulted in a modest increase in apoptosis in MG132-treated proliferating fibroblasts, but had little impact on apoptosis in serum-starved fibroblasts. Because beclin-1 knockdown and Baf inhibit different stages of autophagy via the inhibition of autophagosome formation or the fusion of autophagosomes and lysosomes, respectively, these results suggested that autophagosome formation may be more important in proliferating cells, whereas autophagy/lysosomal activity may be more important in serum-starved cells. Together, these results suggest that autophagy/lysosomal pathways may protect serum-starved fibroblasts from proteasome inhibition-mediated apoptosis and cell death.

Treatment with MG132 increases cellular superoxide levels in proliferating cells and treatment with 2-methoxyestradiol (2-ME) sensitizes serum-starved quiescent fibroblasts to proteasome inhibition

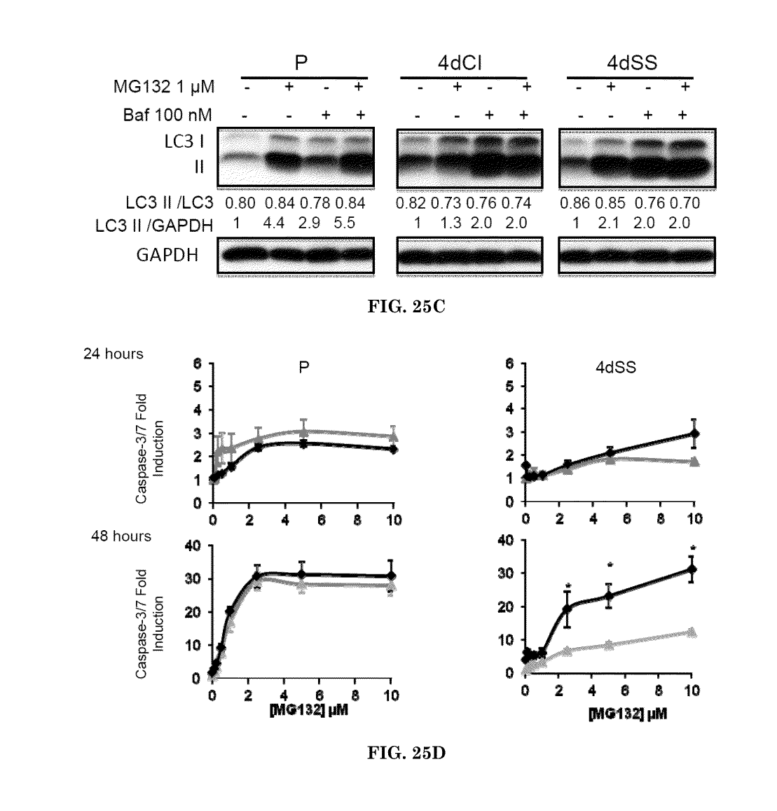

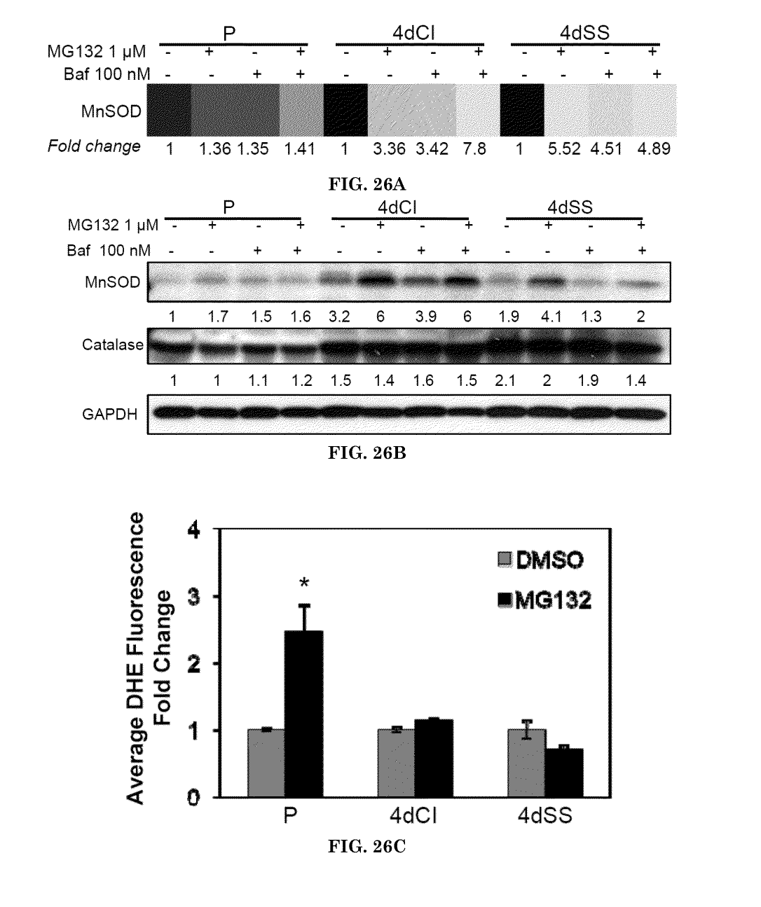

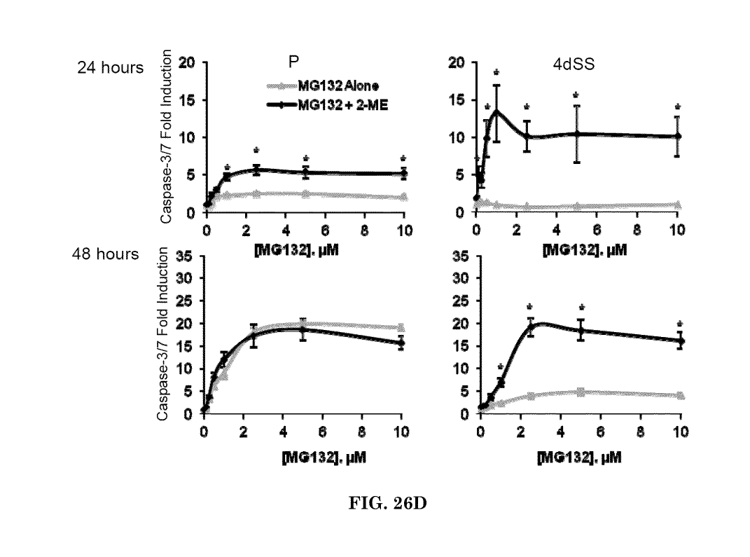

Other researchers have reported that there is a correlation between proliferative status and sensitivity to oxidative stress in human fibroblasts (Naderi et al., 2003, “Oxidative stress-induced apoptosis in dividing fibroblasts involves activation of p38 MAP kinase and over-expression of Bax: resistance of quiescent cells to oxidative stress, Apoptosis 8, 91-100, which is incorporated herein by reference as if fully set forth). Our microarray analysis revealed that treatment with MG132 resulted in the induction of multiple free radical detoxifying gene products including the mitochondrially localized manganese superoxide dismutase, MnSOD, an enzyme that catalyzes the conversion of superoxide into oxygen and hydrogen peroxide (FIG. 26A). Although MG132 treatment induced MnSOD expression in both proliferating and quiescent cells, contact-inhibited and serum-starved cells showed a greater change in MnSOD transcript expression compared to proliferating fibroblasts (FIG. 26A). A Western blot analysis was used to monitor the levels of the ROS detoxifying enzymes MnSOD and catalase, an enzyme that converts hydrogen peroxide to water and oxygen. The protein concentrations of MnSOD and catalase were higher in quiescent fibroblasts than in proliferating cells (FIG. 26B), consistent with a previous report (Sarsour et al., 2008, “Manganese superoxide dismutase activity regulates transitions between quiescent and proliferative growth, Aging cell 7, 405-417, which is incorporated herein by reference as if fully set forth). MG132 treatment further elevated MnSOD levels in both proliferating and quiescent fibroblasts. It was hypothesized that the higher levels of ROS-detoxifying enzymes in quiescent fibroblasts contributes to the protection of quiescent fibroblasts from proteasome inhibition-induced apoptosis. Intracellular superoxide levels were monitored using dihydroethidium (DHE), an indicator of superoxide anions. As shown in FIG. 26C, proteasome inhibition led to significant induction of superoxide levels in proliferating cells while quiescent cells maintained basal superoxide levels regardless of proteasome inhibition. These results suggest a possible role for ROS homeostasis mechanisms in quiescent fibroblasts in response to proteasome inhibition.

It was hypothesized that an improved ability to detoxify free radicals may protect quiescent fibroblasts from proteasome inhibition-mediated apoptosis. To test this, proliferating and serum-starved fibroblasts were treated with 2-methoxyestradiol (2-ME) in the presence of increasing concentrations of MG132, and the induction of apoptosis was monitored. 2-ME treatment has previously been shown to increase cellular superoxide levels in a manner similar to superoxide dismutase (SOD) inhibition; however, the exact mechanism is not clear (Huang et al., 2000, “Superoxide dismutase as a target for the selective killing of cancer cells,” Nature 407, 390-395; Kachadourian et al., 2001, “2-methoxyestradiol does not inhibit superoxide dismutase,” Arch Biochem Biophys 392, 349-353; She et al., 2007, “Requirement of reactive oxygen species generation in apoptosis of leukemia cells induced by 2-methoxyestradiol,” Acta Pharmacol Sin 28, 1037-1044, which are incorporated herein by reference as if fully set forth). 2-ME sensitized serum-starved fibroblasts to MG132-induced apoptosis but had little effect on MG132-treated proliferating fibroblasts (FIG. 26D). This result is consistent with an important role for ROS homeostasis in serum-starved fibroblasts in ensuring cell viability in response to proteasome inhibition.

### EMBODIMENTS

The following list includes particular embodiments of the present invention. The list, however, is not limiting and does not exclude alternate embodiments, as would be appreciated by one of ordinary skill in the art.

1. A composition comprising an autophagy inhibitor and at least one of an NADPH modulator or a glutathione modulator.

2. The composition of embodiment 1, wherein the autophagy inhibitor includes a substance selected from the group consisting of a macrolide antibiotic, bafilomycin, concanamycin, an inhibitor of vacuolar type H+-ATPase, an inhibitor of lysosomal acidification, an antimalarial substance, chloroquine, hydroxychloroquine, micronized hydroxychloroquine, quinacrine, an analog of a macrolide antibiotic, an analog of bafilomycin, chloroquine analogs having a lateral alkyl side chain and dialkyl substitution on the lateral side chain, 7-chloro-N-(3-(4-(7-trifluoromethyl)quinolin-4-yl)piperazin-1-yl)propyl)quinolin-4-amine, {3-[4-(7-chloro-quinolin-4-yl)-piperazin-1-yl]-propyl}-(7-rifluoromethyl-quinolin-4-yl)-amine, 3-methyladenine, an siRNA targeting a protein in the autophagy pathway, an shRNA targeting a protein within the autophagy pathway, an siRNA targeting atg5, an siRNA targeting atg7, an siRNA targeting lc3/atg8, an siRNA targeting beclin1, an shRNA targeting atg5, an shRNA targeting atg7, an shRNA targeting lc3/atg8, and an shRNA targeting beclin 1, or a vector or virus encoding any of the aforementioned peptides, proteins, or RNAs, or an analog or precursor of any of the aforementioned compounds, or a pharmaceutically acceptable salt of any of the foregoing substances.

3. The composition of any one or more of embodiments 1 and 2, wherein the at least one of an NADPH modulator or glutathione modulator includes a substance selected from the group consisting of an inhibitor of glucose-6-phosphate dehydrogenase, an inhibitor of 6 phosphogluconate dehydrogenase, an inhibitor of isocitrate dehydrogenase 1, an inhibitor of isocitrate dehydrogranse 2, an inhibitor of an enzyme in the pentose phosphate pathway, dehydroepiandrosterone, 16α-fluoro-5-androsten-17-one, 16α-fluoro-5α-androstan-17-one, 3-methylandrost-5-en-17-one, somatostatin, a peptide of hypothalamic origin, an inhibitor of transketolase, an analog of a tranketolase inhibitor, a thiamine analog, oxythiamine, a non-charged thiamine analog, a micronized DHEA, DHEA, an siRNA targeting a pentose phosphate pathway enzyme, an siRNA targeting gluocse-6-phosphate dehydrogenase, an siRNA targeting nrf2, an siRNA targeting srbp, an shRNA targeting a pentose phosphate pathway enzyme, an shRNA targeting gluocse-6-phosphate dehydrogenase, an shRNA targeting nrf2, an shRNA targeting srbp, and butathione sulfoximine, or a vector or virus encoding any of the aforementioned peptides, proteins or RNAs, or an analog or precursor of any of the aforementioned compounds, or a pharmaceutically acceptable salt of any of the foregoing substances.

4. The composition of any one or more of the preceding embodiments further comprising an anti-cancer chemotherapeutic agent or a pharmaceutically acceptable salt thereof other than the autophagy inhibitor and the at least one of an NADPH modulator or a glutathione modulator.

5. The composition of any one or more of the preceding embodiments further comprising at least one substance selected from the group consisting of oxaliplatin, capecitabine, bevacizumab, docetaxel, paclitaxel, carboplatin, ixabepilone, androstenedione, testosterone, a precursor of any of the aforementioned compounds and a pharmaceutically acceptable salt of any of the foregoing substances.

6. The composition of any one or more of the preceding embodiments, wherein the NADPH modulator is micronized DHEA or a pharmaceutically acceptable salt thereof, and the autophagy inhibitor is micronized hydroxychloroquine or a pharmaceutically acceptable salt thereof.

7. The composition of any one of more of embodiments 1-5, wherein the NADPH modulator is DHEA.

8. The composition of any one or more of embodiments 1-5 and 7, wherein the autophagy inhibitor is bafilomycin.

9. The composition of any one or more of the preceding embodiments further comprising a targeting agent adapted to deliver at least one of the NADPH modulator or the autophagy inhibitor to a tumor cell.

10. The composition of any one or more of the preceding embodiments further comprising a pharmaceutically acceptable carrier.

11. The composition of embodiment 10, wherein the pharmaceutically acceptable carrier includes at least one substance selected from the group consisting of ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, talc, magnesium carbonate, kaolin, non-ionic surfactants, edible oils, physiological saline, bacteriostatic water, Cremophor EL™ (BASF, Parsippany, N.J.), and phosphate buffered saline (PBS).

12. The composition of any one or more of the preceding embodiments further comprising a reactive oxygen species modulator or a pharmaceutically acceptable salt thereof.

13. The composition of any one or more of the preceding embodiments further comprising a proteasome inhibitor or a pharmaceutically acceptable salt thereof.

14. The composition of any one or more of the preceding embodiments, wherein the proteasome inhibitor is selected from the group consisting of MG132 and bortezomib.

15. The composition of any one or more of the preceding embodiments, wherein the proteasome inhibitor is bortezomib.

16. A method of inhibiting or killing a quiescent cell comprising exposing the quiescent cell to a composition of any one or more of embodiments 1-15.

17. A method of treating cancer comprising administering the composition of any one or more of embodiments 1-15 to a cancer patient.

18. A method of identifying compositions that inhibit or kill quiescent cells comprising:

identifying a target by analyzing at least one of the metabolic flux, gene expression, protein expression, mircoRNA content, histone modification, signaling pathway activity, or physiology of quiescent cells;

identifying a candidate inhibitor of the target; and

exposing a quiescent cell to the candidate inhibitor and identifying whether the candidate inhibitor inhibits or kills quiescent cell.

19. The method of embodiment 18, wherein the step of exposing includes administering the candidate inhibitor to a model organism.

20. The method of any one or more of embodiments 18-19, wherein the step of exposing includes administering the candidate inhibitor to a human.

21. A method of identifying compositions that inhibit or kill quiescent cells comprising exposing a quiescent cell to at least one candidate inhibitor and monitoring the physiology of the quiescent cell.

22. The method of embodiment 21, wherein the step of exposing includes administering the candidate composition to a model organism.

23. The method of any one or more of embodiments 21-22, wherein the step of exposing includes administering the candidate composition to a human.

24. A method of inducing apoptosis comprising exposing at least one of a cell, a cell culture, a tissue, an organ, an organism or a human to a composition comprising the composition of any one or more of embodiments 1-15.

25. A method of sensitizing quiescent cells to proteasome inhibitors comprising exposing at least one of a cell, a cell culture, a tissue, an organ, an organism or a human to a composition comprising the composition of any one or more of embodiments 1-15.

26. A composition comprising DHEA and an autophagy inhibitor.

27. A method of inhibiting or killing a quiescent cell comprising exposing the quiescent cell to DHEA and an autophagy inhibitor.

28. A method of treating cancer comprising administering the composition of embodiment 26 to a cancer patient.

Any of the examples and embodiments herein may be modified by administration of the agents therein to treat at least one of fibrosis, fibrotic tissue or sites that have the potential to develop fibrotic tissue.

Embodiments herein include the compositions utilized in any of the methods described or claimed herein.

Sequences:

| atg5 (NM_004849) | |
|---|---|
| [SEQ ID NO: 23] | |
| 1 | gtgacgtcat ctccgggcgc cgagggtgac tggacttgtg gtgcgctgcc agggctccgc |
| 61 | agcgttgccg gttgtattcg ctggatacca gagggcggaa gtgcagcagg gttcagctcc |
| 121 | gacctccgcg ccggtgcttt ttgcggctgc gcgggcttcc tggagtcctg ctaccgcgtc |
| 181 | cccgcaggac agtgtgtcag gcgggcagct tgccccgccg ccccaccgga gcgcggaatc |
| 241 | tgggcgtccc caccagtgcg gggagccgga aggaggagcc atagcttgga gtaggtttgg |
| 301 | ctttggttga aataagaatt tagcctgtat gtactgcttt aactcctgga agaatgacag |
| 361 | atgacaaaga tgtgcttcga gatgtgtggt ttggacgaat tccaacttgt ttcacgctat |
| 421 | atcaggatga gataactgaa agggaagcag aaccatacta tttgcttttg ccaagagtaa |
| 481 | gttatttgac gttggtaact gacaaagtga aaaagcactt tcagaaggtt atgagacaag |
| 541 | aagacattag tgagatatgg tttgaatatg aaggcacacc actgaaatgg cattatccaa |
| 601 | ttggtttgct atttgatctt cttgcatcaa gttcagctct tccttggaac atcacagtac |
| 661 | attttaagag ttttccagaa aaagaccttc tgcactgtcc atctaaggat gcaattgaag |
| 721 | ctcattttat gtcatgtatg aaagaagctg atgctttaaa acataaaagt caagtaatca |
| 781 | atgaaatgca gaaaaaagat cacaagcaac tctggatggg attgcaaaat gacagatttg |
| 841 | accagttttg ggccatcaat cggaaactca tggaatatcc tgcagaagaa aatggatttc |
| 901 | gttatatccc ctttagaata tatcagacaa cgactgaaag acctttcatt cagaagctgt |
| 961 | ttcgtcctgt ggctgcagat ggacagttgc acacactagg agatctcctc aaagaagttt |
| 1021 | gtccttctgc tattgatcct gaagatgggg aaaaaaagaa tcaagtgatg attcatggaa |
| 1081 | ttgagccaat gttggaaaca cctctgcagt ggctgagtga acatctgagc tacccggata |
| 1141 | attttcttca tattagtatc atcccacagc caacagattg aaggatcaac tatttgcctg |
| 1201 | aacagaatca tccttaaatg ggatttatca gagcatgtca cccttttgct tcaatcaggt |
| 1261 | ttggtggagg caacctgacc agaaacactt cgctgctgca agccagacag gaaaaagatt |
| 1321 | ccatgtcaga taaggcaact gggctggtct tactttgcat cacctctgct ttcctccact |
| 1381 | gccatcatta aacctcagct gtgacatgaa agacttaccg gaccactgaa ggtcttctgt |
| 1441 | aaaatataat gaagctgaaa cctttggcct aagaagaaaa tggaagtatg tgccactcga |
| 1501 | tttgtatttc tgattaacaa ataaacaggg gtatttccta aggtgaccat ggttgaactt |
| 1561 | tagctcatga aagtggaaac attggtttaa ttttcaagag aattaagaaa gtaaaagaga |
| 1621 | aattctgtta tcaataactt gcaagtaatt ttttgtaaaa gattgaatta cagtaaaccc |
| 1681 | atctttcctt aacgaaaatt tcctatgttt acagtctgtc tattggtatg caatcttgta |
| 1741 | actttgataa tgaacagtga gagattttta aataaagcct ctaaatatgt tttgtcattt |
| 1801 | aataacatac agttttgtca cttttcaagt actttctgac tcacatacag tagatcactt |
| 1861 | tttactctgt gttaccattt tgactggtcg tcattggcat ggggtggata tagggcatag |
| 1921 | gattacttgt ctcagaagct gtcatagaat ttcttgctgc caattaaaaa acctgtgttc |
| 1981 | tttacacact acacgtataa atattgtaac tgttcatctt tgttgtttta tcactgtaag |
| 2041 | cctgtcaaat catagtatcc taagcatctg taaatgctaa ttttgcattt ttggaaaaac |
| 2101 | ccattccttc caagctagtg tttttcattg gctccaggtc taatttttca ctgtggtccc |
| 2161 | tggcagccag tcttttgaag tttaaagatt acctgtctct tgactgcagt accttttctt |
| 2221 | taatttttac caaaaatatc cagaggttac tggagttctt attcaatata aggaaagttt |
| 2281 | gctgcacttt attaccaagc ctctgggatt ttaccagtca aacatatttg tgcattacat |
| 2341 | ttcatttctt gtgagctagc tggctgtcca tattgaatgt tgacccattt gagtacgcta |
| 2401 | aaaggcttac agtatcagac acgatcatgg ttttagatcc cataataaaa atgaatgttt |
| 2461 | ttcttataaa aaattataca aatgctgaag tgagattcta ctattgttca ttgcttcctt |
| 2521 | ttctttttcc ttttgcgatt ttcactgatt aatagcacat ttcttcacaa aattagataa |
| 2581 | agttggtcaa agaccagata ttctggaatg gaaattgtaa agcttaatca aaaagaatag |
| 2641 | ccagtacagc atacaatctc agaaacttag aagcaagtag aaaataattg gttgatgtaa |
| 2701 | acgaaagtgc cattttagta aaggcaggaa aaaaatagca atatttgagt tatgtaagga |
| 2761 | taaaaaatcc actgacttgt atttttgcac aagaggctgg tctgaatatg attgttcaca |
| 2821 | ttaagagtgt ttattcgtcg gttcattttg gggattttcc cccttgatgt tttgacagat |
| 2881 | tgaagtgagc tttagtgagc aaaaggatca gaatgcaggg aacactaagc tgtgatgaag |
| 2941 | aaagtgtggt aaaaagccag agtagtttta tacagacaaa accagtgtca ggcctttgca |
| 3001 | gtaggcttga gtgaacttct gatctagatt tgaaagtaaa ttttatgaag acattgccca |
| 3061 | tttttacttc ctcattcatt attgtaccag catcatagct ttattactct aatcccaggt |
| 3121 | aagtcaagcc tacaatgccc tagaggaaga gtaaaaccag aaattcatgc tggcttaaat |
| 3181 | aatctatttt tgtttctttt catttgaata tttaaatttt atggtttatt aaaaaattaa |
| 3241 | ataa |
| atg7 (NM_006395) | |
| [SEQ ID NO: 24] | |
| 1 | ctttgcgcac gcgcgccgct tcccagtggc aagcgcgggc aggaccgcgt tgcgtcatcg |
| 61 | gggcgcgcgc ctcagagaga gctgtggttg ccggaagttg agcggcggca agaaataatg |
| 121 | gcggcagcta cgggggatcc tggactctct aaactgcagt ttgccccttt tagtagtgcc |
| 181 | ttggatgttg ggttttggca tgagttgacc cagaagaagc tgaacgagta tcggctggat |
| 241 | gaagctccca aggacattaa gggttattac tacaatggtg actctgctgg gctgccagct |
| 301 | cgcttaacat tggagttcag tgcttttgac atgagtgctc ccaccccagc ccgttgctgc |
| 361 | ccagctattg gaacactgta taacaccaac acactcgagt ctttcaagac tgcagataag |
| 421 | aagctccttt tggaacaagc agcaaatgag atatgggaat ccataaaatc aggcactgct |
| 481 | cttgaaaacc ctgtactcct caacaagttc ctcctcttga catttgcaga tctaaagaag |
| 541 | taccacttct actattggtt ttgctatcct gccctctgtc ttccagagag tttacctctc |
| 601 | attcaggggc cagtgggttt ggatcaaagg ttttcactaa aacagattga agcactagag |
| 661 | tgtgcatatg ataatctttg tcaaacagaa ggagtcacag ctcttcctta cttcttaatc |
| 721 | aagtatgatg agaacatggt gctggtttcc ttgcttaaac actacagtga tttcttccaa |
| 781 | ggtcaaagga cgaagataac aattggtgta tatgatccct gtaacttagc ccagtaccct |
| 841 | ggatggcctt tgaggaattt tttggtccta gcagcccaca gatggagtag cagtttccag |
| 901 | tctgttgaag ttgtttgctt ccgtgaccgt accatgcagg gggcgagaga cgttgcccac |
| 961 | agcatcatct tcgaagtgaa gcttccagaa atggcattta gcccagattg tcctaaagca |
| 1021 | gttggatggg aaaagaacca gaaaggaggc atgggaccaa ggatggtgaa cctcagtgaa |
| 1081 | tgtatggacc ctaaaaggtt agctgagtca tcagtggatc taaatctcaa actgatgtgt |
| 1141 | tggagattgg ttcctacttt agacttggac aaggttgtgt ctgtcaaatg tctgctgctt |
| 1201 | ggagccggca ccttgggttg caatgtagct aggacgttga tgggttgggg cgtgagacac |
| 1261 | atcacatttg tggacaatgc caagatctcc tactccaatc ctgtgaggca gcctctctat |
| 1321 | gagtttgaag attgcctagg gggtggtaag cccaaggctc tggcagcagc ggaccggctc |
| 1381 | cagaaaatat tccccggtgt gaatgccaga ggattcaaca tgagcatacc tatgcctggg |
| 1441 | catccagtga acttctccag tgtcactctg gagcaagccc gcagagatgt ggagcaactg |
| 1501 | gagcagctca tcgaaagcca tgatgtcgtc ttcctattga tggacaccag ggagagccgg |
| 1561 | tggcttcctg ccgtcattgc tgcaagcaag agaaagctgg tcatcaatgc tgctttggga |
| 1621 | tttgacacat ttgttgtcat gagacatggt ctgaagaaac caaagcagca aggagctggg |
| 1681 | gacttgtgtc caaaccaccc tgtggcatct gctgacctcc tgggctcatc gctttttgcc |
| 1741 | aacatccctg gttacaagct tggctgctac ttctgcaatg atgtggtggc cccaggagat |
| 1801 | tcaaccagag accggacctt ggaccagcag tgcactgtga gtcgtccagg actggccgtg |
| 1861 | attgcaggag ccctggccgt ggaattgatg gtatctgttt tgcagcatcc agaagggggc |
| 1921 | tatgccattg ccagcagcag tgacgatcgg atgaatgagc ctccaacctc tcttgggctt |
| 1981 | gtgcctcacc agatccgggg atttctttca cggtttgata atgtccttcc cgtcagcctg |
| 2041 | gcatttgaca aatgtacagc ttgttcttcc aaagttcttg atcaatatga acgagaagga |
| 2101 | tttaacttcc tagccaaggt gtttaattct tcacattcct tcttagaaga cttgactggt |
| 2161 | cttacattgc tgcatcaaga aacccaagct gctgagatct gggacatgag cgatgatgag |
| 2221 | accatctgag atggccccgc tgtggggctg acttctcccc ggccgcctgc tgaggagctc |
| 2281 | tccatcgcca gagcaggact gctgacccca ggcctggtga ttctgggccc ctcctccata |
| 2341 | ccccgaggtc tgggattccc ccctctgctg cccaggagtg gccagtgttc ggcgttgctc |
| 2401 | gggattcaag ataccaccag ttcagagcta aataataacc ttggccttgg ccttgctatt |
| 2461 | gacctgggac ttggtcctcc atgcagtttt tatttcttgt cacagtgact gatagccatc |
| 2521 | ccccaggatc ctttcccctt ggccctgagg gggtgaccca acacagacca aatggggaaa |
| 2581 | tgagcaacca gctcctgccc agagccactg cgggaggtgg caccctcatc cccggaatgt |
| 2641 | gctgcccacc gcaccgcagg ctcctcctgt gggggccctg ggcatgggtg agggtgggac |
| 2701 | cccgtgagcg cactgcaccc tggccctggt ggagcgggag gaggaggaga gccgagctgg |
| 2761 | gtacgagact aaagggccca catgacccag tgacgccaga tttccaccaa ggactgagtg |
| 2821 | agctgctcag acatggcttt ctgcctccca gcctgtcctc cactgtgggc atagcatctg |
| 2881 | tgcctgcctg cctgcttgag ggagaggagt ttctgctgct gccttgagct ggggggaaga |
| 2941 | gcccaggggc agatcctggc agctgcctgg atggggctcc tccctgccct tatgagcagg |
| 3001 | ccaggcccag aaaggccgag cctgggctgc cttcctgccc cagccgaggg aggggtcaga |
| 3061 | cggctctacc atgggtaact caggcaagag ctggttttcc tctttattct gggtgtgtgc |
| 3121 | agctgtgagg ccccaaccca ggagaggcca tggcctaggt acctgtgacc accctgcccc |
| 3181 | cgtgtagagg gcatcgtctt tcctgctatt ttattctttc agcttttgtc ttaggcccag |
| 3241 | aatcaaagtg aaaattgagt cgagctgacc cttacaacag taggatttag tagggtagat |
| 3301 | ttcaaatgag gcttcgcttc tcccaaagta gccagtccaa gttccagtgg ctgtcgttca |
| 3361 | gctcatggga gcttcatggg gacacagccg gcacaggtgc agggcccgag tccgcccacc |
| 3421 | cagcctggcg ctgaaactgc acacgtacac tatgtggttt aagagcactt tattattgtt |
| 3481 | cttaaggcta cttttaagta caaaaaaaga tggcctgcca aacctttttt tttcttcttc |
| 3541 | caggaaaaac aggccacaga gaatggtata ttacagattt acacacatga agagaaggtc |
| 3601 | agagcgcact gcaggcagcg cggctctggg aagaacttca cggagcccct tcttagagca |
| 3661 | gggagggggc tttctcagtg aaatgtttgg ttttctgctg cctcctctgc cccaggcccc |
| 3721 | cctccagggt actgcctatc ccagataggt cagtgcacca gggacccggc cgccagcacc |
| 3781 | gccgacccct cccagagtga cgcccttgtt cactgacaaa gagacctgtc ccaggagtgt |
| 3841 | cctccaccga gccggtcagc tgtgggtggt tttcctgtta cgacgctcag tagcctgtag |
| 3901 | caataacaaa ctcgtggcta tgaatgcaga tgcagtgttc tcatagaata actgttcctg |
| 3961 | cacttttaca gacaaatcta cgacaaaaaa aaagatcaac tttttttttc cgaacaacaa |
| 4021 | aaaaaatgaa tgattacaat aggaaaggga aaaattaaat agctacatat cattaacaaa |
| 4081 | ttaatgttct tcaaaaaata cctacaaatt tctctgtaca ttctttacgc acagcgtaac |
| 4141 | gatggtctca aaatcaccca tatagaaaag tgttctcaac gatttttcct acagaaaata |
| 4201 | taggggcctg aatgccaaag cttggaagcc cagtacagtg ggagtgaaat gtgtgcgggg |
| 4261 | caaggagaag ggcttttctt tcctccactt ttcaaaggcc tgcagccact ctgtgactac |
| 4321 | aagagccagt cctccgacct tttcacccag tgccaatttc caaaattcaa cagctaaaaa |
| 4381 | ctgtaaaacc gggggtcata cggtgtgcag agtccacaaa gccttgcagg tgaggtgacc |
| 4441 | acgcccacgt cacctggtca ggtgccatcg tcgtgagcct ctggtgggcc aggtgggaca |
| 4501 | cagcacaccc cagggggagg ggatagaaac gctcattgac caaaaaggag cagctgtgac |
| 4561 | ctccacagct gtgtctgtca tgcttgcttc atctaatttc tagttagtag ctattaatat |
| 4621 | agcaaataat aaatgcagta ataacagtat aaagtcagag gaatgtatac tgccttggcc |
| 4681 | ccagcgtacg aggaagcgta taaaacacca tatcacagat tgtctgtcag taatctgctg |
| 4741 | ttcagccaag agagttcaaa gggagcagtt tctgcatgta gggaagttgg aagacacaaa |
| 4801 | ccccacctcc cctgggagct tgtaacaaag cagacaggga tgcaaaaata aatgatgtca |
| 4861 | gcctgcagcc aaactccagc atcccacacc gcagctgacc cactgctcat cgcgagggcc |
| 4921 | tgccaggagc tggcctcccg cactacttgt gagtaaagtg aatatcaaat accaatctta |
| 4981 | gagtacaact gtaccagcag taagtatatc taggactgta actgacaaaa ataaactaat |
| 5041 | tctgaaaaga aaaaaaaaa |
| beclin1 (NM_003766) | |
| [SEQ ID NO: 25] | |
| 1 | ggaagttttc cggcggctac cgggaagtcg ctgaagacag agcgatggta gttctggagg |
| 61 | cctcgctccg gggccgaccc gaggccacag tgcctccgcg gtagaccgga cttgggtgac |
| 121 | gggctccggg ctcccgaggt gaagagcatc gggggctgag ggatggaagg gtctaagacg |
| 181 | tccaacaaca gcaccatgca ggtgagcttc gtgtgccagc gctgcagcca gcccctgaaa |
| 241 | ctggacacga gtttcaagat cctggaccgt gtcaccatcc aggaactcac agctccatta |
| 301 | cttaccacag cccaggcgaa accaggagag acccaggagg aagagactaa ctcaggagag |
| 361 | gagccattta ttgaaactcc tcgccaggat ggtgtctctc gcagattcat ccccccagcc |
| 421 | aggatgatgt ccacagaaag tgccaacagc ttcactctga ttggggaggc atctgatggc |
| 481 | ggcaccatgg agaacctcag ccgaagactg aaggtcactg gggacctttt tgacatcatg |
| 541 | tcgggccaga cagatgtgga tcacccactc tgtgaggaat gcacagatac tcttttagac |
| 601 | cagctggaca ctcagctcaa cgtcactgaa aatgagtgtc agaactacaa acgctgtttg |
| 661 | gagatcttag agcaaatgaa tgaggatgac agtgaacagt tacagatgga gctaaaggag |
| 721 | ctggcactag aggaggagag gctgatccag gagctggaag acgtggaaaa gaaccgcaag |
| 781 | atagtggcag aaaatctcga gaaggtccag gctgaggctg agagactgga tcaggaggaa |
| 841 | gctcagtatc agagagaata cagtgaattt aaacgacagc agctggagct ggatgatgag |
| 901 | ctgaagagtg ttgaaaacca gatgcgttat gcccagacgc agctggataa gctgaagaaa |
| 961 | accaacgtct ttaatgcaac cttccacatc tggcacagtg gacagtttgg cacaatcaat |
| 1021 | aacttcaggc tgggtcgcct gcccagtgtt cccgtggaat ggaatgagat taatgctgct |
| 1081 | tggggccaga ctgtgttgct gctccatgct ctggccaata agatgggtct gaaatttcag |
| 1141 | agataccgac ttgttcctta cggaaaccat tcatatctgg agtctctgac agacaaatct |
| 1201 | aaggagctgc cgttatactg ttctgggggg ttgcggtttt tctgggacaa caagtttgac |
| 1261 | catgcaatgg tggctttcct ggactgtgtg cagcagttca aagaagaggt tgagaaaggc |
| 1321 | gagacacgtt tttgtcttcc ctacaggatg gatgtggaga aaggcaagat tgaagacaca |
| 1381 | ggaggcagtg gcggctccta ttccatcaaa acccagttta actctgagga gcagtggaca |
| 1441 | aaagctctca agttcatgct gacgaatctt aagtggggtc ttgcttgggt gtcctcacaa |
| 1501 | ttttataaca aatgactttt ttccttaggg ggaggtttgc cttaaaggct tttaattttg |
| 1561 | ttttgtttgc aaacatgttt taaattaaat tcgggtaata ttaaacagta catgtttaca |
| 1621 | ataccaaaaa agaaaaaatc cacaaaagcc actttatttt aaaatatcat gtgacagata |
| 1681 | ctttccagag ctacaacatg ccatctatag ttgccagccc tggtcagttt tgattcttaa |
| 1741 | ccccatggac tcctttccct ttcttctctg aaaaaaacta atttaaattt gcttttcttt |
| 1801 | tttttaactg agttgaattg agattgatgt gttttcactg gatttttatc tctctcaact |
| 1861 | tcctgcactt aacaatatga aatagaaact tttgtcttta ctgagatgag gatatgtttg |
| 1921 | agatgcacag ttggataatg tgggaaaatg acatctaagc tttacctggt caccatgtga |
| 1981 | tgtgatcaga tgcttgaaat ttaacacttt tcacttggtt cttatactga atgccgactc |
| 2041 | tgctctgtgt tagagatatg aaatggtgtt tgatactgtt tgagacatta tggagagatt |
| 2101 | taattatttg taataaaaga tttgctgcag tctgaaaact gcc |

The references cited throughout this application, are incorporated for all purposes apparent herein and in the references themselves as if each reference was fully set forth. For the sake of presentation, specific ones of these references are cited at particular locations herein. A citation of a reference at a particular location indicates a manner(s) in which the teachings of the reference are incorporated. However, a citation of a reference at a particular location does not limit the manner in which all of the teachings of the cited reference are incorporated for all purposes.

It is understood, therefore, that this invention is not limited to the particular embodiments disclosed, but is intended to cover all modifications which are within the spirit and scope of the invention as defined by the appended claims; the above description; and/or shown in the attached drawings.

What is claimed is:

1. A composition comprising an autophagy inhibitor and at least one of an NADPH modulator or a glutathione modulator.

2. The composition of claim 1, wherein the autophagy inhibitor includes a substance selected from the group consisting of a macrolide antibiotic, bafilomycin, concanamycin, an inhibitor of vacuolar type H+-ATPase, an inhibitor of lysosomal acidification, an antimalarial substance, chloroquine, hydroxychloroquine, micronized hydroxychloroquine, quinacrine, an analog of a macrolide antibiotic, an analog of bafilomycin, chloroquine analogs having a lateral alkyl side chain and dialkyl substitution on the lateral side chain, 7-chloro-N-(3-(4-(7-trifluoromethyl)quinolin-4-yl)piperazin-1-yl)propyl)quinolin-4-amine, {3-[4-(7-chloro-quinolin-4-yl)-piperazin-1-yl]-propyl}-(7-rifluoromethyl-quinolin-4-yl)-amine, 3-methyladenine, an siRNA targeting a protein in the autophagy pathway, an shRNA targeting a protein within the autophagy pathway, an siRNA targeting atg5, an siRNA targeting atg7, an siRNA targeting lc3/atg8, an siRNA targeting beclin1, an shRNA targeting atg5, an shRNA targeting atg7, an shRNA targeting lc3/atg8, and an shRNA targeting beclin 1, or a vector or virus encoding any of the aforementioned peptides, proteins, or RNAs, or an analog or precursor of any of the aforementioned compounds, or a pharmaceutically acceptable salt of any of the foregoing substances.

3. The composition of claim 1, wherein the at least one of an NADPH modulator or a glutathione modulator includes a substance selected from the group consisting of an inhibitor of glucose-6-phosphate dehydrogenase, an inhibitor of 6 phosphogluconate dehydrogenase, an inhibitor of isocitrate dehydrogenase 1, an inhibitor of isocitrate dehydrogranse 2, an inhibitor of an enzyme in the pentose phosphate pathway, dehydroepiandrosterone, 16α-fluoro-5-androsten-17-one, 16α-fluoro-5α-androstan-17-one, 3-β-methylandrost-5-en-17-one, somatostatin, a peptide of hypothalamic origin, an inhibitor of transketolase, an analog of a tranketolase inhibitor, a thiamine analog, oxythiamine, a non-charged thiamine analog, a micronized DHEA, DHEA, an siRNA targeting a pentose phosphate pathway enzyme, an siRNA targeting gluocse-6-phosphate dehydrogenase, an siRNA targeting nrf2, an siRNA targeting srbp, an shRNA targeting a pentose phosphate pathway enzyme, an shRNA targeting gluocse-6-phosphate dehydrogenase, an shRNA targeting nrf2, an shRNA targeting srbp, and butathione sulfoximine, or a vector or virus encoding any of the aforementioned peptides, proteins, or RNAs, or an analog or precursor of any of the aforementioned compounds, or a pharmaceutically acceptable salt of any of the foregoing substances.

4. The composition of claim 1 further comprising an anti-cancer chemotherapeutic agent or a pharmaceutically acceptable salt thereof other than the autophagy inhibitor and other than the at least one of an NADPH modulator or a glutathione modulator.

5. The composition of any of claim 1 further comprising at least one substance selected from the group consisting of oxaliplatin, capecitabine, bevacizumab, docetaxel, paclitaxel, carboplatin, ixabepilone, androstenedione, testosterone, a precursor of any of the aforementioned compounds and a pharmaceutically acceptable salt of any of the foregoing substances.

6. The composition of claim 1, wherein the at least one of an NADPH modulator or a glutathione modulator is micronized DHEA or a pharmaceutically acceptable salt thereof, and the autophagy inhibitor is micronized hydroxychloroquine or a pharmaceutically acceptable salt thereof.

7. The composition of claim 1, wherein the at least one of an NADPH modulator or a glutathione modulator is DHEA.

8. The composition of claim 1, wherein the autophagy inhibitor is bafilomycin.

9. The composition of claim 1 further comprising a targeting agent adapted to deliver the at least one of an NADPH modulator or a glutathione modulator or the autophagy inhibitor to a tumor cell.

10. The composition of claim 1 further comprising a pharmaceutically acceptable carrier.

11. The composition of claim 10, wherein the pharmaceutically acceptable carrier includes at least one substance selected from the group consisting of ion exchangers, alumina, aluminum stearate, lecithin, serum proteins, human serum albumin, buffer substances, phosphates, glycine, sorbic acid, potassium sorbate, partial glyceride mixtures of saturated vegetable fatty acids, water, salts, electrolytes, protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, waxes, polyethylene glycol, starch, lactose, dicalcium phosphate, microcrystalline cellulose, sucrose, talc, magnesium carbonate, kaolin, non-ionic surfactants, edible oils, physiological saline, bacteriostatic water, Cremophor EL™ (BASF, Parsippany, N.J.), and phosphate buffered saline (PBS).

12. The composition of claim 1 further comprising a reactive oxygen species modulator or a pharmaceutically acceptable salt thereof.

13. The composition of claim 1 further comprising a proteasome inhibitor or a pharmaceutically acceptable salt thereof.

14. The composition of claim 13, wherein the proteasome inhibitor is selected from the group consisting of MG132 and bortezomib.

15. The composition of claim 13, wherein the proteasome inhibitor is bortezomib.

16. A method of inhibiting or killing a quiescent cell comprising exposing the quiescent cell to at an autophagy inhibitor and at least one of an NADPH modulator or a glutathione modulator.

17. The method of claim 16, wherein the inhibitor of autophagy includes a substance selected from the group consisting of a macrolide antibiotic, bafilomycin, concanamycin, an inhibitor of vacuolar type H+-ATPase, an inhibitor of lysosomal acidification, an antimalarial substance, chloroquine, hydroxychloroquine, micronized hydroxychloroquine, quinacrine, an analog of a macrolide antibiotic, an analog of bafilomycin, chloroquine analogs having a lateral alkyl side chain and dialkyl substitution on the lateral side chain, 7-chloro-N-(3-(4-(7-trifluoromethyl)quinolin-4-yl)piperazin-1-yl)propyl)quinolin-4-amine, {3-[4-(7-chloro-quinolin-4-yl)-piperazin-1-yl]-propyl}-(7-rifluoromethyl-quinolin-4-yl)-amine, an siRNA targeting a protein in the autophagy pathway, an shRNA targeting a protein within the autophagy pathway, an siRNA targeting atg5, an siRNA targeting atg7, an siRNA targeting lc3/atg8, an siRNA targeting beclin1, an shRNA targeting atg5, an shRNA targeting atg7, an shRNA targeting lc3/atg8, and an shRNA targeting beclin 1, or a vector or virus encoding any of the aforementioned peptides, proteins, or RNAs, or an analog or precursor of any of the aforementioned compounds, or a pharmaceutically acceptable salt of any of the foregoing substances.

18. The method of claim 16, wherein the at least one of an NADPH modulator or a glutathione modulator includes a substance selected from the group consisting of an inhibitor of glucose-6-phosphate dehydrogenase, an inhibitor of 6 phosphogluconate dehydrogenase, an inhibitor of isocitrate dehydrogenase 1, an inhibitor of isocitrate dehydrogranse 2, an inhibitor of an enzyme in the pentose phosphate pathway, dehydroepiandrosterone, 16α-fluoro-5-androsten-17-one, 16α-fluoro-5α-androstan-17-one, 3-β-methylandrost-5-en-17-one, somatostatin, a peptide of hypothalamic origin, an inhibitor of transketolase, an analog of a tranketolase inhibitor, a thiamine analog, oxythiamine, a non-charged thiamine analog, a micronized DHEA, DHEA, an siRNA targeting a pentose phosphate pathway enzyme, an siRNA targeting gluocse-6-phosphate dehydrogenase, an siRNA targeting nrf2, an siRNA targeting srbp, an shRNA targeting a pentose phosphate pathway enzyme, an shRNA targeting gluocse-6-phosphate dehydrogenase, an shRNA targeting nrf2, an shRNA targeting srbp, and butathione sulfoximine, or a vector or virus encoding any of the aforementioned peptides, proteins, or RNAs, or an analog or precursor of any of the aforementioned compounds, or a pharmaceutically acceptable salt of any of the foregoing substances.

19. The method of claim 16 further comprising administering a reactive oxygen species modulator.

20. The method of claim 16 further comprising administering a proteasome inhibitor.

21. A method of treating cancer comprising administering an autophagy inhibitor and at least one of an NADPH modulator or a glutathione modulator to a cancer patient.

22. The method of claim 21, wherein the inhibitor of autophagy includes a substance selected from the group consisting of a macrolide antibiotic, bafilomycin, concanamycin, an inhibitor of vacuolar type H+-ATPase, an inhibitor of lysosomal acidification, an antimalarial substance, chloroquine, hydroxychloroquine, micronized hydroxychloroquine, quinacrine, an analog of a macrolide antibiotic, an analog of bafilomycin, chloroquine analogs having a lateral alkyl side chain and dialkyl substitution on the lateral side chain, 7-chloro-N-(3-(4-(7-trifluoromethyl)quinolin-4-yl)piperazin-1-yl)propyl)quinolin-4-amine, {3-[4-(7-chloro-quinolin-4-yl)-piperazin-1-yl]-propyl}-(7-rifluoromethyl-quinolin-4-yl)-amine, an siRNA targeting a protein in the autophagy pathway, an shRNA targeting a protein within the autophagy pathway, an siRNA targeting atg5, an siRNA targeting atg7, an siRNA targeting lc3/atg8, an siRNA targeting beclin1, an shRNA targeting atg5, an shRNA targeting atg7, an shRNA targeting lc3/atg8, and an shRNA targeting beclin 1, or a vector or virus encoding any of the aforementioned peptides, proteins, or RNAs, or an analog or precursor of any of the aforementioned compounds, or a pharmaceutically acceptable salt of any of the foregoing substances.

23. The method of claim 21, wherein the at least one of an NADPH modulator or a glutathione modulator includes a substance selected from the group consisting of an inhibitor of glucose-6-phosphate dehydrogenase, an inhibitor of 6 phosphogluconate dehydrogenase, an inhibitor of isocitrate dehydrogenase 1, an inhibitor of isocitrate dehydrogranse 2, an inhibitor of an enzyme in the pentose phosphate pathway, dehydroepiandrosterone, 16α-fluoro-5-androsten-17-one, 16α-fluoro-5α-androstan-17-one, 3-β-methylandrost-5-en-17-one, somatostatin, a peptide of hypothalamic origin, an inhibitor of transketolase, an analog of a tranketolase inhibitor, a thiamine analog, oxythiamine, a non-charged thiamine analog, a micronized DHEA, DHEA, an siRNA targeting a pentose phosphate pathway enzyme, an siRNA targeting gluocse-6-phosphate dehydrogenase, an siRNA targeting nrf2, an siRNA targeting srbp, an shRNA targeting a pentose phosphate pathway enzyme, an shRNA targeting gluocse-6-phosphate dehydrogenase, an shRNA targeting nrf2, an shRNA targeting srbp, and butathione sulfoximine or a vector or virus encoding any of the aforementioned peptides, proteins, or RNAs, or an analog or precursor of any of the aforementioned compounds, or a pharmaceutically acceptable salt of any of the foregoing substances.

24. The method of claim 21 further comprising administering a reactive oxygen species modulator.

25. The method of claim 21 further comprising administering a proteasome inhibitor.

26. The method of claim 21 further comprising administering an anti-cancer chemotherapeutic agent or a pharmaceutically acceptable salt thereof other than the autophagy inhibitor and other than the at least one of an NADPH modulator or a glutathione modulator.

27. The method of claim 26, wherein the anti-cancer chemotherapeutic agent includes at least one substance selected from the group consisting of oxaliplatin, capecitabine, bevacizumab, docetaxel, paclitaxel, carboplatin, ixabepilone, androstenedione, and testosterone, or a pharmaceutically acceptable salt of any of the foregoing substances.

28. A method of identifying compositions that inhibit or kill quiescent cells comprising:
identifying a target by analyzing at least one of the metabolic flux, gene expression, protein expression, mircoRNA content, histone modification, signaling pathway activity, or physiology of quiescent cells;
identifying a candidate inhibitor of the target; and
exposing a quiescent cell to the candidate inhibitor and identifying whether the candidate inhibitor inhibits or kills the quiescent cell.

29. The method of claim 28, wherein the step of exposing includes administering the candidate inhibitor to a model organism.

30. The method of claim 28, wherein the step of exposing includes administering the candidate inhibitor to a human.

31. A method of identifying compositions that inhibit or kill quiescent cells comprising exposing a quiescent cell to at least one candidate inhibitor and monitoring the physiology of the quiescent cell.

32. The method of claim 31, wherein the step of exposing includes administering the at least one candidate inhibitor to a model organism.

33. The method of claim 31, wherein the step of exposing includes administering the at least one candidate inhibitor to a human.

34. A method of inducing apoptosis comprising exposing at least one of a cell, a cell culture, a tissue, an organ, an organism or a human to a composition comprising an autophagy inhibitor and at least one of an NADPH modulator or a glutathione modulator.

35. The method of claim 34, wherein the composition further comprises a reactive oxygen species modulator.

36. The method of claim 34, wherein the composition further comprises a proteasome inhibitor.

37. A method of sensitizing quiescent cells to proteasome inhibitors comprising exposing a cell, a cell culture, a tissue, an organ, an organism or a human to an autophagy inhibitor and at least one of an NADPH modulator or a glutathione modulator.

38. A composition comprising DHEA and an autophagy inhibitor.

39. A method of inhibiting or killing a quiescent cell comprising exposing the quiescent cell to DHEA and an autophagy inhibitor.

40. A method of treating cancer comprising administering DHEA and an autophagy inhibitor to a cancer patient.


SEQUENCE LISTING (25 sequences)

SEQ ID NO: 1 - RNA, 97 nt, Homo sapiens
ugcuguugacagugagcgaggacaacaucgccugcguuauuagugaagccacagauguaauaacgcaggcgauguugucccugccuacugccucgga

SEQ ID NO: 2 - DNA, 23 bp, Homo sapiens
gugagauaugguuugaauadtdt

SEQ ID NO: 3 - DNA, 23 bp, Homo sapiens
uauucaaaccauaucucacdtdt

SEQ ID NO: 4 - DNA, 23 bp, Homo sapiens
gauaugguuugaauaugaadtdt

SEQ ID NO: 5 - DNA, 23 bp, Homo sapiens
uucauauucaaaccauaucdtdt

SEQ ID NO: 6 - DNA, 21 bp, Homo sapiens
cctgaacagaatcatccttaa

SEQ ID NO: 7 - DNA, 60 bp, Homo sapiens
gccggcctgaacagaatcatccttaactcgagttaaggatgattctgttcaggttttttg

SEQ ID NO: 8 - DNA, 21 bp, Homo sapiens
cctgaacagaatcatccttaa

SEQ ID NO: 9 - DNA, 58 bp, Homo sapiens
ccggcctgaacagaatcatccttaactcgagttaaggatgattctgttcaggtttttg

SEQ ID NO: 10 - DNA, 23 bp, Homo sapiens
caguuacagauggagcuaadtdt

SEQ ID NO: 11 - DNA, 23 bp, Homo sapiens
uuagcuccaucuguaacugdtdt

SEQ ID NO: 12 - DNA, 23 bp, Homo sapiens
gagauaugggaauccauaadtdt

SEQ ID NO: 13 - DNA, 23 bp, Homo sapiens
uuauggauucccauaucucdtdt

SEQ ID NO: 14 - DNA, 23 bp, Homo sapiens
cagcuauuggaacacuguadtdt

SEQ ID NO: 15 - DNA, 23 bp, Homo sapiens
uacaguguuccaauagcugdtdt

SEQ ID NO: 16 - DNA, 21 bp, Homo sapiens
gcctgctgaggagctctccat

SEQ ID NO: 17 - DNA, 57 bp, Homo sapiens
ccgggcctgctgaggagctctccatctcgagatggagagctcctcagcaggcttttt

SEQ ID NO: 18 - DNA, 21 bp, Homo sapiens
cccagctattggaacactgta

SEQ ID NO: 19 - DNA, 57 bp, Homo sapiens
ccggcccagctattggaacactgtactcgagtacagtgttccaatagctgggttttt

SEQ ID NO: 20 - RNA, 138 nt, Homo sapiens
cagaaggcucgagaagguauauugcuguugacagugagcgagacaguuuggcacaaucaauauagugaagccacagauguauauugauugugccaaacuguccugccuacugccucggaauucaaggggcuacuuuag

SEQ ID NO: 21 - DNA, 23 bp, Homo sapiens
guuuggagaucuuagagcadtdt

SEQ ID NO: 22 - DNA, 23 bp, Homo sapiens
ugcucuaagaucuccaaacdtdt

SEQ ID NO: 23 - DNA, 3244 bp, Homo sapiens
gtgacgtcatctccgggcgccgagggtgactggacttgtggtgcgctgccagggctccgcagcgttgccggttgtattcgctggataccagagggcggaagtgcagcagggttcagctccgacctccgcgccggtgctttttgcggctgcgcgggcttcctggagtcctgctaccgcgtccccgcaggacagtgtgtcaggcgggcagcttgccccgccgccccaccggagcgcggaatctgggcgtccccaccagtgcggggagccggaaggaggagccatagcttggagtaggtttggctttggttgaaataagaatttagcctgtatgtactgctttaactcctggaagaatgacagatgacaaagatgtgcttcgagatgtgtggtttggacgaattccaacttgtttcacgctatatcaggatgagataactgaaagggaagcagaaccatactatttgcttttgccaagagtaagttatttgacgttggtaactgacaaagtgaaaaagcactttcagaaggttatgagacaagaagacattagtgagatatggtttgaatatgaaggcacaccactgaaatggcattatccaattggtttgctatttgatcttcttgcatcaagttcagctcttccttggaacatcacagtacattttaagagttttccagaaaaagaccttctgcactgtccatctaaggatgcaattgaagctcattttatgtcatgtatgaaagaagctgatgctttaaaacataaaagtcaagtaatcaatgaaatgcagaaaaaagatcacaagcaactctggatgggattgcaaaatgacagatttgaccagttttgggccatcaatcggaaactcatggaatatcctgcagaagaaaatggatttcgttatatcccctttagaatatatcagacaacgactgaaagacctttcattcagaagctgtttcgtcctgtggctgcagatggacagttgcacacactaggagatctcctcaaagaagtttgtccttctgctattgatcctgaagatggggaaaaaaagaatcaagtgatgattcatggaattgagccaatgttggaaacacctctgcagtggctgagtgaacatctgagctacccggataattttcttcatattagtatcatcccacagccaacagattgaaggatcaactatttgcctgaacagaatcatccttaaatgggatttatcagagcatgtcacccttttgcttcaatcaggtttggtggaggcaacctgaccagaaacacttcgctgctgcaagccagacaggaaaaagattccatgtcagataaggcaactgggctggtcttactttgcatcacctctgctttcctccactgccatcattaaacctcagctgtgacatgaaagacttaccggaccactgaaggtcttctgtaaaatataatgaagctgaaacctttggcctaagaagaaaatggaagtatgtgccactcgatttgtatttctgattaacaaataaacaggggtatttcctaaggtgaccatggttgaactttagctcatgaaagtggaaacattggtttaattttcaagagaattaagaaagtaaaagagaaattctgttatcaataacttgcaagtaattttttgtaaaagattgaattacagtaaacccatctttccttaacgaaaatttcctatgtttacagtctgtctattggtatgcaatcttgtaactttgataatgaacagtgagagatttttaaataaagcctctaaatatgttttgtcatttaataacatacagttttgtcacttttcaagtactttctgactcacatacagtagatcactttttactctgtgttaccattttgactggtcgtcattggcatggggtggatatagggcataggattacttgtctcagaagctgtcatagaatttcttgctgccaattaaaaaacctgtgttctttacacactacacgtataaatattgtaactgttcatctttgttgttttatcactgtaagcctgtcaaatcatagtatcctaagcatctgtaaatgctaattttgcatttttggaaaaacccattccttccaagctagtgtttttcattggctccaggtctaatttttcactgtggtccctggcagccagtcttttgaagtttaaagattacctgtctcttgactgcagtaccttttctttaatttttaccaaaaatatccagaggttactggagttcttattcaatataaggaaagtttgctgcactttattaccaagcctctgggattttaccagtcaaacatatttgtgcattacatttcatttcttgtgagctagctggctgtccatattgaatgttgacccatttgagtacgctaaaaggcttacagtatcagacacgatcatggttttagatcccataataaaaatgaatgtttttcttataaaaaattatacaaatgctgaagtgagattctactattgttcattgcttccttttctttttccttttgcgattttcactgattaatagcacatttcttcacaaaattagataaagttggtcaaagaccagatattctggaatggaaattgtaaagcttaatcaaaaagaatagccagtacagcatacaatctcagaaacttagaagcaagtagaaaataattggttgatgtaaacgaaagtgccattttagtaaaggcaggaaaaaaatagcaatatttgagttatgtaaggataaaaaatccactgacttgtatttttgcacaagaggctggtctgaatatgattgttcacattaagagtgtttattcgtcggttcattttggggattttcccccttgatgttttgacagattgaagtgagctttagtgagcaaaaggatcagaatgcagggaacactaagctgtgatgaagaaagtgtggtaaaaagccagagtagttttatacagacaaaaccagtgtcaggcctttgcagtaggcttgagtgaacttctgatctagatttgaaagtaaattttatgaagacattgcccatttttacttcctcattcattattgtaccagcatcatagctttattactctaatcccaggtaagtcaagcctacaatgccctagaggaagagtaaaaccagaaattcatgctggcttaaataatctatttttgtttcttttcatttgaatatttaaattttatggtttattaaaaaattaaataa

SEQ ID NO: 24 - DNA, 5059 bp, Homo sapiens
ctttgcgcacgcgcgccgcttcccagtggcaagcgcgggcaggaccgcgttgcgtcatcggggcgcgcgcctcagagagagctgtggttgccggaagttgagcggcggcaagaaataatggcggcagctacgggggatcctggactctctaaactgcagtttgccccttttagtagtgccttggatgttgggttttggcatgagttgacccagaagaagctgaacgagtatcggctggatgaagctcccaaggacattaagggttattactacaatggtgactctgctgggctgccagctcgcttaacattggagttcagtgcttttgacatgagtgctcccaccccagcccgttgctgcccagctattggaacactgtataacaccaacacactcgagtctttcaagactgcagataagaagctccttttggaacaagcagcaaatgagatatgggaatccataaaatcaggcactgctcttgaaaaccctgtactcctcaacaagttcctcctcttgacatttgcagatctaaagaagtaccacttctactattggttttgctatcctgccctctgtcttccagagagtttacctctcattcaggggccagtgggtttggatcaaaggttttcactaaaacagattgaagcactagagtgtgcatatgataatctttgtcaaacagaaggagtcacagctcttccttacttcttaatcaagtatgatgagaacatggtgctggtttccttgcttaaacactacagtgatttcttccaaggtcaaaggacgaagataacaattggtgtatatgatccctgtaacttagcccagtaccctggatggcctttgaggaattttttggtcctagcagcccacagatggagtagcagtttccagtctgttgaagttgtttgcttccgtgaccgtaccatgcagggggcgagagacgttgcccacagcatcatcttcgaagtgaagcttccagaaatggcatttagcccagattgtcctaaagcagttggatgggaaaagaaccagaaaggaggcatgggaccaaggatggtgaacctcagtgaatgtatggaccctaaaaggttagctgagtcatcagtggatctaaatctcaaactgatgtgttggagattggttcctactttagacttggacaaggttgtgtctgtcaaatgtctgctgcttggagccggcaccttgggttgcaatgtagctaggacgttgatgggttggggcgtgagacacatcacatttgtggacaatgccaagatctcctactccaatcctgtgaggcagcctctctatgagtttgaagattgcctagggggtggtaagcccaaggctctggcagcagcggaccggctccagaaaatattccccggtgtgaatgccagaggattcaacatgagcatacctatgcctgggcatccagtgaacttctccagtgtcactctggagcaagcccgcagagatgtggagcaactggagcagctcatcgaaagccatgatgtcgtcttcctattgatggacaccagggagagccggtggcttcctgccgtcattgctgcaagcaagagaaagctggtcatcaatgctgctttgggatttgacacatttgttgtcatgagacatggtctgaagaaaccaaagcagcaaggagctggggacttgtgtccaaaccaccctgtggcatctgctgacctcctgggctcatcgctttttgccaacatccctggttacaagcttggctgctacttctgcaatgatgtggtggccccaggagattcaaccagagaccggaccttggaccagcagtgcactgtgagtcgtccaggactggccgtgattgcaggagccctggccgtggaattgatggtatctgttttgcagcatccagaagggggctatgccattgccagcagcagtgacgatcggatgaatgagcctccaacctctcttgggcttgtgcctcaccagatccggggatttctttcacggtttgataatgtccttcccgtcagcctggcatttgacaaatgtacagcttgttcttccaaagttcttgatcaatatgaacgagaaggatttaacttcctagccaaggtgtttaattcttcacattccttcttagaagacttgactggtcttacattgctgcatcaagaaacccaagctgctgagatctgggacatgagcgatgatgagaccatctgagatggccccgctgtggggctgacttctccccggccgcctgctgaggagctctccatcgccagagcaggactgctgaccccaggcctggtgattctgggcccctcctccataccccgaggtctgggattcccccctctgctgcccaggagtggccagtgttcggcgttgctcgggattcaagataccaccagttcagagctaaataataaccttggccttggccttgctattgacctgggacttggtcctccatgcagtttttatttcttgtcacagtgactgatagccatcccccaggatcctttccccttggccctgagggggtgacccaacacagaccaaatggggaaatgagcaaccagctcctgcccagagccactgcgggaggtggcaccctcatccccggaatgtgctgcccaccgcaccgcaggctcctcctgtgggggccctgggcatgggtgagggtgggaccccgtgagcgcactgcaccctggccctggtggagcgggaggaggaggagagccgagctgggtacgagactaaagggcccacatgacccagtgacgccagatttccaccaaggactgagtgagctgctcagacatggctttctgcctcccagcctgtcctccactgtgggcatagcatctgtgcctgcctgcctgcttgagggagaggagtttctgctgctgccttgagctggggggaagagcccaggggcagatcctggcagctgcctggatggggctcctccctgcccttatgagcaggccaggcccagaaaggccgagcctgggctgccttcctgccccagccgagggaggggtcagacggctctaccatgggtaactcaggcaagagctggttttcctctttattctgggtgtgtgcagctgtgaggccccaacccaggagaggccatggcctaggtacctgtgaccaccctgcccccgtgtagagggcatcgtctttcctgctattttattctttcagcttttgtcttaggcccagaatcaaagtgaaaattgagtcgagctgacccttacaacagtaggatttagtagggtagatttcaaatgaggcttcgcttctcccaaagtagccagtccaagttccagtggctgtcgttcagctcatgggagcttcatggggacacagccggcacaggtgcagggcccgagtccgcccacccagcctggcgctgaaactgcacacgtacactatgtggtttaagagcactttattattgttcttaaggctacttttaagtacaaaaaaagatggcctgccaaacctttttttttcttcttccaggaaaaacaggccacagagaatggtatattacagatttacacacatgaagagaaggtcagagcgcactgcaggcagcgcggctctgggaagaacttcacggagccccttcttagagcagggagggggctttctcagtgaaatgtttggttttctgctgcctcctctgccccaggcccccctccagggtactgcctatcccagataggtcagtgcaccagggacccggccgccagcaccgccgacccctcccagagtgacgcccttgttcactgacaaagagacctgtcccaggagtgtcctccaccgagccggtcagctgtgggtggttttcctgttacgacgctcagtagcctgtagcaataacaaactcgtggctatgaatgcagatgcagtgttctcatagaataactgttcctgcacttttacagacaaatctacgacaaaaaaaaagatcaactttttttttccgaacaacaaaaaaaatgaatgattacaataggaaagggaaaaattaaatagctacatatcattaacaaattaatgttcttcaaaaaatacctacaaatttctctgtacattctttacgcacagcgtaacgatggtctcaaaatcacccatatagaaaagtgttctcaacgatttttcctacagaaaatataggggcctgaatgccaaagcttggaagcccagtacagtgggagtgaaatgtgtgcggggcaaggagaagggcttttctttcctccacttttcaaaggcctgcagccactctgtgactacaagagccagtcctccgaccttttcacccagtgccaatttccaaaattcaacagctaaaaactgtaaaaccgggggtcatacggtgtgcagagtccacaaagccttgcaggtgaggtgaccacgcccacgtcacctggtcaggtgccatcgtcgtgagcctctggtgggccaggtgggacacagcacaccccagggggaggggatagaaacgctcattgaccaaaaaggagcagctgtgacctccacagctgtgtctgtcatgcttgcttcatctaatttctagttagtagctattaatatagcaaataataaatgcagtaataacagtataaagtcagaggaatgtatactgccttggccccagcgtacgaggaagcgtataaaacaccatatcacagattgtctgtcagtaatctgctgttcagccaagagagttcaaagggagcagtttctgcatgtagggaagttggaagacacaaaccccacctcccctgggagcttgtaacaaagcagacagggatgcaaaaataaatgatgtcagcctgcagccaaactccagcatcccacaccgcagctgacccactgctcatcgcgagggcctgccaggagctggcctcccgcactacttgtgagtaaagtgaatatcaaataccaatcttagagtacaactgtaccagcagtaagtatatctaggactgtaactgacaaaaataaactaattctgaaaagaaaaaaaaaa

SEQ ID NO: 25 - DNA, 2143 bp, Homo sapiens
ggaagttttccggcggctaccgggaagtcgctgaagacagagcgatggtagttctggaggcctcgctccggggccgacccgaggccacagtgcctccgcggtagaccggacttgggtgacgggctccgggctcccgaggtgaagagcatcgggggctgagggatggaagggtctaagacgtccaacaacagcaccatgcaggtgagcttcgtgtgccagcgctgcagccagcccctgaaactggacacgagtttcaagatcctggaccgtgtcaccatccaggaactcacagctccattacttaccacagcccaggcgaaaccaggagagacccaggaggaagagactaactcaggagaggagccatttattgaaactcctcgccaggatggtgtctctcgcagattcatccccccagccaggatgatgtccacagaaagtgccaacagcttcactctgattggggaggcatctgatggcggcaccatggagaacctcagccgaagactgaaggtcactggggacctttttgacatcatgtcgggccagacagatgtggatcacccactctgtgaggaatgcacagatactcttttagaccagctggacactcagctcaacgtcactgaaaatgagtgtcagaactacaaacgctgtttggagatcttagagcaaatgaatgaggatgacagtgaacagttacagatggagctaaaggagctggcactagaggaggagaggctgatccaggagctggaagacgtggaaaagaaccgcaagatagtggcagaaaatctcgagaaggtccaggctgaggctgagagactggatcaggaggaagctcagtatcagagagaatacagtgaatttaaacgacagcagctggagctggatgatgagctgaagagtgttgaaaaccagatgcgttatgcccagacgcagctggataagctgaagaaaaccaacgtctttaatgcaaccttccacatctggcacagtggacagtttggcacaatcaataacttcaggctgggtcgcctgcccagtgttcccgtggaatggaatgagattaatgctgcttggggccagactgtgttgctgctccatgctctggccaataagatgggtctgaaatttcagagataccgacttgttccttacggaaaccattcatatctggagtctctgacagacaaatctaaggagctgccgttatactgttctggggggttgcggtttttctgggacaacaagtttgaccatgcaatggtggctttcctggactgtgtgcagcagttcaaagaagaggttgagaaaggcgagacacgtttttgtcttccctacaggatggatgtggagaaaggcaagattgaagacacaggaggcagtggcggctcctattccatcaaaacccagtttaactctgaggagcagtggacaaaagctctcaagttcatgctgacgaatcttaagtggggtcttgcttgggtgtcctcacaattttataacaaatgacttttttccttagggggaggtttgccttaaaggcttttaattttgttttgtttgcaaacatgttttaaattaaattcgggtaatattaaacagtacatgtttacaataccaaaaaagaaaaaatccacaaaagccactttattttaaaatatcatgtgacagatactttccagagctacaacatgccatctatagttgccagccctggtcagttttgattcttaaccccatggactcctttccctttcttctctgaaaaaaactaatttaaatttgcttttcttttttttaactgagttgaattgagattgatgtgttttcactggatttttatctctctcaacttcctgcacttaacaatatgaaatagaaacttttgtctttactgagatgaggatatgtttgagatgcacagttggataatgtgggaaaatgacatctaagctttacctggtcaccatgtgatgtgatcagatgcttgaaatttaacacttttcacttggttcttatactgaatgccgactctgctctgtgttagagatatgaaatggtgtttgatactgtttgagacattatggagagatttaattatttgtaataaaagatttgctgcagtctgaaaactgcc